(19) United States
(12) Patent Application Publication
(10) Pub. No.: US 2015/0202304 A1
(43) Pub. Date: Jul. 23, 2015

(54) ENCAPSULATION OF IMMISCIBLE PHASES IN SILK FIBROIN BIOMATERIALS

(71) Applicant: TUFTS UNIVERSITY, Medford, MA (US)

(72) Inventors: David L. Kaplan, Concord, MA (US); Fiorenzo Omenetto, Lexington, MA (US); Eleanor M. Pritchard, New Orleans, LA (US)

(21) Appl. No.: 14/414,218

(22) PCT Filed: Jul. 15, 2013
(86) PCT No.: PCT/US2013/050529

§ 371 Date: Jan. 12, 2015

Related U.S. Application Data
(60) Provisional application No. 61/671,336, filed on Jul. 13, 2012.
(60) Provisional application No. 61/791,185, filed on Mar. 15, 2013.

(51) Int. Cl. A61K 47/42; A61K 9/107; A61K 8/11; A23L 1/48; A61K 31/7076; A61K 31/355; A61K 31/20; A61K 31/015; A61K 9/70; A61K 8/64

(52) U.S. Cl. CPC A61K 47/42; A61K 9/7007; A61K 9/1075; A61K 8/11; A61K 8/64; A61K 31/7076; A61K 31/355; A61K 31/20; A61K 31/015; A23L 1/48; A23V 2002/00

(57) ABSTRACT

Embodiments of various aspects described herein relates to compositions and methods for encapsulation and/or stabilization of oil, lipid, hydrophobic and/or lipophilic compounds in a silk-based material. The compositions described herein can be used in various applications, e.g., pharmaceutical, cosmetic, food, diagnostic, and tissue engineering applications.

### CROSS REFERENCE TO RELATED APPLICATIONS

This application claims the benefit under 35 U.S.C. §119(e) of U.S. Provisional Application Nos. 61/671,336 filed Jul. 13, 2012 and 61/791,185 filed Mar. 15, 2013, the content of each of which is incorporated herein by reference in its entirety.

### TECHNICAL FIELD

Described herein generally relates to compositions and methods for encapsulation and/or stabilization of oil, lipid, hydrophobic or lipophilic compounds including active agents in a biocompatible matrix.

### BACKGROUND

Previously used in the pharmaceutical industry to improve drug bioavailability, stabilize drugs against various degradation pathways, minimize side effects or modify drug release kinetics, encapsulation or microencapsulation techniques have also been discussed to be used in other fields, for example, food (Gibbs et al., 1999; Madene et al., 2006) and fragrances (Berthier et al., 2010; Ouali et al., 2006). Microencapsulation is generally a process in which tiny particles or droplets are surrounded by a protective coating layer, or embedded within an encapsulating matrix or membrane, providing a physical barrier between the incorporated compound and the surrounding environment (Baranauskiene et al., 2006; Madene et al., 2006; Gharsallaoui et al., 2007; Sohail et al., 2011).

Various encapsulation materials and techniques have been previously reported (see, e.g., Gouin, 2004; Gibbs et al., 1999; Gharsallaoui et al., 2007; Madene et al., 2006; Kuang et al., 2010). Some biopolymers discussed to be used as an encapsulating matrix include, for example, natural gums (e.g., gum arabic, alginates, carragenans), proteins (e.g., milk or whey proteins, gelatin), maltodextrins with different dextrose equivalences, and waxes and their blends (Gharsallaoui et al., 2007). Proteins can be used as encapsulant materials because their physicochemical properties (including, e.g., amphiphilic character, ability to self-associate and interact with a variety of substances, high molecular weight, and molecular chain flexibility) can provide various functional properties for encapsulation (including, e.g., solubility, viscosity, emulsification and film-formation) (Madene et al., 2006; Gharsallaoui et al., 2007; Baranauskiene et al., 2006; Dickinson, 2011). During emulsion formation, protein molecules can act as emulsifiers by rapidly adsorbing at the newly formed oil-water interface, forming a steric-stabilizing layer (Arshady et al., 1990; Madene et al., 2006; Dickinson, 2011). However, use of proteins as encapsulant materials for certain applications can be challenging. For example, gelatin suffers from serious drawbacks that limit its widespread use. Gelatin is highly viscous even in low concentrations, possesses low solubility in cold water, and gluteraldehyde (the chemical used to cross-link gelatin) is toxic to humans (Jun-xia et al., 2011). In addition, concerns regarding the safety of animal-derived proteins have increased in response to the recent emergence of diseases such as the prions (Chourpa et al., 2006).

Further, various existing encapsulation approaches require processing conditions which can degrade delicate compounds and/or compromise the safety of the final product (such as exposure to high heat or the use of toxic cross-linking chemicals (Liu et al., 1996; Qian et al., 1997; Demura et al., 1989; Lu et al., 2010)). For example, protein microspheres were discussed to be prepared from water-in-oil emulsions where aqueous protein solutions were dispersed in an oil bath (sometimes stabilized with emulsifiers and/or surfactants), then the proteins were stabilized via suspension crosslinking, either through thermal or chemical treatment (Arshady, 1990; Jayakrishnan et al., 1994; Esposito et al., 1996; Imsombut et al., 2010). Imsombut et al. have prepared silk microspheres using this method, with ethyl acetate as the oil phase, Span80 as an oil-soluble emulsifier, and genipin as a crosslinker (Imsombut et al., 2010). However, a process devoid of chemical additives is preferable as the chemical additives can have toxic side-effects in vivo or damage delicate compounds (Esposito et al., 1996). Proteins droplets in water-in-oil emulsions were discussed to be converted to microparticles without chemical treatment by heating the oil bath to crosslink the protein matrix (Arshady, 1990; Esposito et al., 1996). However, heating is preferred to be avoided given the temperature-sensitive nature of many active agents (Jun-xia et al., 2011; Kanakdande et al., 2007). Accordingly, there is still an unmet need for development of novel encapsulation techniques that can reduce the loss of labile molecules (e.g., volatile and/or lipophilic molecules), sustain the presence of these labile molecules in consumer products, and/or protect and stabilize these labile molecules.

### SUMMARY

Various existing encapsulation approaches require processing conditions which can degrade labile molecules (e.g., volatile, hydrophobic, and/or lipophilic molecules) and/or compromise the safety and/or efficacy of the final product (such as exposure to high heat or the use of toxic crosslinking chemicals). Hence, there is still an unmet need for novel encapsulation techniques that can improve the encapsulation efficiency of labile molecules (e.g., volatile, hydrophobic, and/or lipophilic molecules), protect and stabilize these labile molecules, and/or controllably release these labile molecules.

The inventors have inter alia demonstrated novel techniques for encapsulating oil in silk biomaterials using emulsion-based processes that exploit unique properties of silk, including, e.g., amphiphilicity, biocompatibility, aqueous and ambient processing and tunable physical crosslinking behavior. For example, in some embodiments, sonication-induced self-assembly of silk to fabrication of silk hydrogels, for example, as described in U.S. Pat. No. 8,187,616, was used in place of the thermal or chemical suspension crosslinking that is traditionally used to stabilize the aqueous protein phase in emulsions of water in oil (W/O) and oil in water in oil (O/W/O) type. Stable silk micro- and macro-particles loaded with oil (optionally containing oil-soluble active agent(s)) or loaded with water-soluble active agent(s) were produced, for example, by sonicating a silk solution (optionally comprising oil droplets) and aliquoting the sonicated silk solution into an oil bath. It was discovered that oil microdroplets are stably emulsified in aqueous silk solutions without addition of any emulsifiers and the presence of oil microdroplets did not impede self-assembly of silk into solid-state silk materials such as films or hydrogel networks. The inventors have further demonstrated that, in O/W/O emulsions, particle morphology and the permeability of the silk to a lipophilic active agent in the interior oil phase (or release of the lipophilic active agent from the interior oil phase to a surrounding) were determined at least in part by silk solution concentration, silk processing and/or sonication. These stable emulsions of oil phase in silk biomaterials (oil-encapsulated silk biomaterials) can be used in various applications, e.g., in tissue engineering such as to model a tissue with a high lipid content, as well as for delivery and/or stabilization/storage of an active agent that are soluble in the oil phase of the silk biomaterials such as therapeutic agents, diagnostic agent, food additives, lipids, and cosmetically active agents.

Accordingly, embodiments of various aspects provided herein relate to compositions comprising an emulsion of an immiscible phase (e.g., an oil phase) dispersed in a silk-based material, as well as methods of making and uses of the compositions. In some embodiments, the immiscible phase can contain at least one oil-soluble, hydrophobic or lipophilic active agent. In some embodiments, in order to produce a stable emulsion of oil droplets in aqueous silk, the silk mixture can be subjected to sonication. In these embodiments, the sonicated silk solution containing a dispersion of oil droplets can be further introduced into an oil bath to form oil-loaded silk particles (e.g., silk particles encapsulating one or more oil droplets).

In one aspect, provided herein relates to silk-based emulsion compositions. The composition comprises at least two immiscible phases, a first immiscible phase comprising a silk-based material and a second immiscible phase comprising an active agent, wherein the first immiscible phase encapsulates the second immiscible phase (or stated another way, the second immiscible phase is dispersed in the first immiscible phase) and the second immiscible phases excludes a liposome.

In some embodiments, the second immiscible phase can comprise a lipid component, e.g., but not limited to, oil, fatty acids, glycerolipids, glycerophospholipids, sphingolipids, saccharolipids, polyketides, sterol lipids and prenol lipids. In some embodiments, the lipid component can exclude phospholipids. In some embodiments, the lipid component can exclude glycerophospholipids. In one embodiment, the lipid component is oil.

The second immiscible phase can form a single or a plurality of (e.g., at least two or more) droplets of any size and/or shape. The size and/or shape of the droplets can vary with a number of factors including, e.g., silk solution concentration and/or silk processing. In some embodiments, the size of the droplets can be in a range of about 1 nm to about 1000 μm, or about 5 nm to about 500 μm.

Any active agent that is preferentially soluble in the second immiscible phase can be included in the second immiscible phase. In some embodiments, the active agent present in the second immiscible phase is a volatile, hydrophobic and/or lipophilic molecule. Examples of the volatile, hydrophobic and/or lipophilic molecule include, without limitations, a therapeutic agent, a nutraceutical agent, a cosmetic agent, a coloring agent, a probiotic agent, a dye, an aromatic compound, an aliphatic compound (e.g., but not limited to, alkane, alkene, alkyne, a cycloaliphatic compound such as cyclo-alkane, cyclo-alkene, and cyclo-alkyne), a small molecule, and any combinations thereof.

In some embodiments, the second immiscible phase can further encapsulate a third immiscible phase, e.g., an aqueous phase.

The first immiscible phase (comprising a silk-based material) can be solid/or gel-like when the second immiscible phase can be liquid. Alternatively, the first immiscible phase (comprising a silk-based material) can be solid/gel-like when the second immiscible phase can be solid/gel-like. In some embodiments, the aqueous phase can comprise pores and the oil phase can occupy at least one of the pores.

The volumetric ratio of the second immiscible phase (e.g., lipid droplets) to the first immiscible phase (e.g., a silk-based material) can vary with the emulsion configuration, silk solution concentration, silk processing, sonication treatment, and/or applications of the composition. In some embodiments, the volumetric ratio of the lipid droplets to the silk-based material can range from about 1000:1 to about 1:1000, from about 500:1 to about 1:500, from about 100:1 to about 1:100, or form about 10:1 to about 1:10.

The first immiscible phase comprises a silk-based material. The silk-based material can be soluble or insoluble in an aqueous medium. The solubility of the silk-based material in an aqueous medium can be controlled by the beta-sheet content in silk fibroin. For example, the beta-sheet content in silk fibroin can be increased by exposing the silk-based material to a post-treatment that increases beta-sheet formation to an amount sufficient to enable a silk-based material to resist dissolution in an aqueous medium.

In some embodiments, the first immiscible phase can further comprise an additive and/or a second active agent. In some embodiments, the additive and/or the second active agent can be incorporated into the silk-based material. The second active agent can be any agent that is preferentially soluble in the first immiscible phase.

Non-limiting examples of the additive that can be added into the first immiscible phase include biocompatible polymers; plasticizers (e.g., glycerol); stimulus-responsive agents; emulsifiers or emulsion stabilizers (e.g., polyvinyl alcohol, and lecithin), surfactants (e.g., polysorbate-20), interfacial tension-reducing agents (e.g., salt), beta-sheet inducing agents (e.g., salt), detectable agents, small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. Depending on the form of the silk-based material, the additive can be present in any form, e.g., including, but not limited to, a particle (e.g., a nanoparticle or microparticle, including a plasmonic particle), a fiber, a tube, a film, a gel, a mesh, a mat, a non-woven mat, a powder or any combinations thereof. In some embodiments, the additive can comprise a silk material, e.g., but not limited to, silk particles, silk fibers, micro-sized silk fibers, unprocessed silk fibers, and any combinations thereof.

In some embodiments, the silk-based material can comprise an optical pattern on at least one of its surface. For example, the optical pattern can comprise a hologram or an array of patterns that provides an optical functionality, e.g., but not limited to, light reflection, diffraction, scattering, iridescence, and any combinations thereof.

The silk-based material can be present in any form or shape. For example, the silk-based material can be in a form of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized material, a porous material, a non-porous material, or any combinations thereof. In some embodiments, the silk-based material can be present in a hydrated state (e.g., as a hydrogel). In some embodiments, the silk-based material can be present in a dried state, e.g., by drying under an ambient condition and/or by lyophilization. In some embodiments, the lyophilized silk-based material can be porous.

In some embodiments, the silk-based material can form a film.

In some embodiments, the silk-based material can form a scaffold.

In some embodiments, the silk-based material can form a particle. Accordingly, other aspects provided herein relate to a silk particle comprising an emulsion of lipid droplets and compositions comprising the silk particle. In one aspect, provided herein is a silk particle comprising at least two immiscible phases, a first immiscible phase comprising silk fibroin and a second immiscible phase comprising an active agent, wherein the first immiscible phase encapsulates the second immiscible phase (or stated another way, the second immiscible phase is dispersed in the first immiscible phase) and the second immiscible phases excludes a liposome.

The silk particle can be of any size. For example, the size of the silk particle can range from about 10 nm to about 10 mm, or from about 50 nm to about 5 mm.

The second immiscible phase can form a single or a plurality of (e.g., at least two or more) droplets of any size and/or shape in the silk particle. The size and/or shape of the droplets can vary with a number of factors including, e.g., silk solution concentration, silk processing, and/or size of the silk particle. In some embodiments, the size of the droplets can be in a range of about 1 nm to about 1000 μm, or about 5 nm to about 500 μm.

Compositions comprising a plurality of (e.g., at least two or more) one or more embodiments of the silk particles are also provided herein. Depending on intended uses (e.g., but not limited to, a pharmaceutical product, a cosmetic product, a personal care product, and a food product), the compositions can be formulated to form an emulsion, a colloid, a cream, a gel, a lotion, a paste, an ointment, a liniment, a balm, a liquid, a solid (e.g., wax), a film, a sheet, a fabric, a mesh, a sponge, an aerosol, powder, a scaffold, or any combinations thereof.

In accordance with various aspects described herein, silk can act as an emulsifier to stabilize an emulsion of lipid droplets dispersed in a silk-based material. Further, silk can stabilize an active agent encapsulated therein as described in International Pat. App. No. WO 2012/145739, the content of which is incorporated herein by reference. Accordingly, a further aspect provided herein relates to a storage-stable silk-based emulsion composition. The storage-stable comprises a silk-based emulsion composition described herein or a silk particle described herein, wherein the active agent (e.g., a volatile, hydrophobic, and/or lipophilic agent) present in the second immiscible phase (e.g., lipid droplets) of the composition or the silk particle retains at least about 30% of its original bioactivity and/or original loading after the composition is (a) subjected to at least one freeze-thaw cycle, or (b) maintained for at least about 24 hours at about room temperature or above, or (c) both (a) and (b).

The storage-stable compositions described herein can protect the active agent from deactivation and/or degradation due to temperature fluctuation and/or eliminate the need for refrigeration. In some embodiments, the storage-stable composition described herein can also stabilize the active agent when it is exposed to light or a relative humidity of at least about 10% or more. Thus, in some embodiments, the active agent (e.g., a volatile, hydrophobic, and/or lipophilic agent) present in the second immiscible phase (e.g., lipid droplets) of the composition or the silk particle can retain at least about 30% of its original bioactivity and/or original loading after the composition is also maintained under exposure to light. In some embodiments, the active agent (e.g., a volatile, hydrophobic, and/or lipophilic agent) present in the second immiscible phase (e.g., lipid droplets) of the composition or the silk particle can retain at least about 30% of its original bioactivity and/or original loading after the composition is also maintained at a relative humidity of at least about 10% or more.

In some embodiments, the silk-based material or the silk particle can be present in a dried-state or a lyophilized state.

The lipid-droplet(s)-loaded silk particles described herein can be produced by any methods known in the art. For example, in some embodiments, hollow silk particles can be produced, e.g., using the phase separation method as described in International Patent App. No. WO 2011/041395, or the lipid-template guided fabrication method as described in International Patent App. No. WO 2008/118133, followed by immersion in an oil solution for loading/diffusion of oil into the silk particles. In some embodiments, an emulsion of lipid droplets in an aqueous silk solution can be subjected to a freeze-dry process. In some embodiments, the lipid-droplet(s)-loaded silk particles can be produced by a novel fabrication process as presented herein, which can be controlled to produce a silk particle encapsulating one or more lipid droplets therein.

The novel process of making a lipid-droplet(s)-loaded silk particle described herein comprises (a) providing an emulsion of non-aqueous droplets dispersed in a silk solution undergoing a sol-gel transition (where the silk solution remains in a mixable state); and (b) adding a pre-determined volume of the emulsion into a non-aqueous phase, thereby the silk solution forms in the non-aqueous phase a silk particle entrapping at least one of the non-aqueous droplets therein.

In some embodiments, the emulsion in step (a) can be produced by adding a non-aqueous, immiscible phase into the silk solution, thereby forming an emulsion of non-aqueous droplets dispersed in the silk solution. In some embodiments, the non-aqueous droplets can further comprise a volatile, hydrophobic, and/or lipophilic molecule as described herein. In these embodiments, the volatile, hydrophobic and/or lipophilic molecule can be added to the non-aqueous immiscible phase prior to forming the emulsion.

The sol-gel transition of the silk solution can last for any period of time as long as the silk solution comprising the non-aqueous droplets still remains in the solution state when it is aliquoted into a non-aqueous phase, e.g., an oil phase, and then can form a gel particle in the non-aqueous phase. In some embodiments, the sol-gel transition can last for at least about 15 minutes or more, including, e.g., at least about 30 minutes, at least about 1 hour, at least about 2 hours or more.

While the sol-gel transition of the silk solution can be induced by any methods known in the art, including, e.g., sonication, shear stress, electrogelation, pH reduction, salt addition, air-drying, water annealing, water vapor annealing, alcohol immersion, or any combination thereof, in one embodiment, the sol-gel transition of the silk solution can be induced by sonication. In some embodiments, the sonication can be performed at an amplitude of about 1% to about 50%, or about 5% to about 25%, or about 10% to about 15%. In some embodiments, the sonication duration can last for from about 5 sec to about 90 sec, or from about 15 sec to about 60 sec, or from about 30 sec to about 45 sec. The sonication treatment parameters (e.g., amplitude, time, or both) can be controlled accordingly to adjust for the desirable material properties of the resulting silk particles (e.g., silk particle size and/or shape, lipid droplet size and/or shape, and/or permeability of the silk as an encapsulant material.

In addition to the sonication treatment parameters, other control parameters for the material properties of the silk particles include, e.g., but not limited to, silk solution properties (e.g., composition, concentration, solution viscosity, silk degumming time), particle fabrication parameters (e.g., presence or absence of particle coating(s), volumetric ratio of silk fibroin and lipid phase, and aliquot volume of a silk-based emulsion (dispersion of lipid droplets in the sol-gel silk solution) added to a continuous phase (e.g., an oil phase)), post-treatment of the silk particle (e.g., but not limited to beta-sheet inducing treatment such as lyophilization, water annealing, and water vapor annealing), if any, and any combinations thereof.

By way of example only, the concentration of the silk solution can, in part, influence the lipid encapsulation configuration. For example, higher concentrations of the silk solution can produce a dispersion of multiple oil droplets suspended throughout the silk-comprising phase (termed as “a microsphere”), while lower concentrations of the silk solution can result in a “microcapsule” configuration, where one large lipid droplet surrounded by a silk capsule is incorporated in each individual particle. Accordingly, the silk solution can have a concentration of about 0.5% (w/v) to about 30%(w/v), about 1% (w/v) to about 15% (w/v), or about 2% (w/v) to about 7% (w/v).

In some embodiments, the sol-gel silk solution can further comprise an active agent as described herein.

By adding a pre-determined volume of the emulsion from step (a) into the non-aqueous phase (e.g., an oil phase), e.g., dropwise via an extrusion-like process, the size of the resulting silk particle can be controlled. For example, the pre-determined volume of the emulsion can substantially correspond or proportional to a desirable size of the silk particle.

In some embodiments, the method can further comprise isolating the formed silk particle from the non-aqueous phase.

In some embodiments, the method can further comprise subjecting the silk particle to a post-treatment. The post-treatment can include any process that changes at least one material property of the silk particle. For example, in some embodiments, the post-treatment can include a dehydration process (e.g., by drying or lyophilization) to produce a silk particle in a dried state. In some embodiments, lyophilization of the silk particle can introduce porous structure in silk matrix therein. In other embodiments, the post-treatment can include a process that further induces a conformational change in silk fibroin in the particle. The conformational change in silk fibroin can be induced, for example, but not limited to, one or more of lyophilization or freeze-drying, water annealing, water vapor annealing, alcohol immersion, sonication, shear stress, electrogelation, pH reduction, salt addition, air-drying, electrospinning, stretching, or any combination thereof.

Different embodiments of the compositions described herein can be used, for example, in tissue engineering such as to model a tissue with high lipid content, or in controlled release and/or stabilization of a volatile, hydrophobic and/or lipophilic agent as described herein. Accordingly, methods of using one or more embodiments of the compositions are also provided herein. For example, some embodiments of the compositions described herein can be used to stabilize an active agent present in the second immiscible phase of the composition (e.g., a volatile, hydrophobic and/or lipophilic agent present in an interior oil phase). Thus, in one aspect, the method of use can comprise maintaining at least one composition (including a storage-stable composition described herein) or at least one silk particle described herein, and wherein the active agent present in the second immiscible phase of the composition or the silk particle can retain at least about 30% of its original bioactivity and/or original loading after the composition is (a) subjected to at least one freeze-thaw cycle, or (b) maintained for at least about 24 hours or longer at about room temperature or above, or (c) both (a) and (b). In some embodiments, the composition can be maintained for at least about 1 month or longer.

Additionally or alternatively, some embodiments of the compositions described herein can be used to controllably release an active agent from the second immiscible phase of the composition (e.g., a volatile, hydrophobic and/or lipophilic agent present in an interior oil phase). Thus, in one aspect, the method of use can comprise maintaining at least one composition (including a storage-stable composition described herein) or at least one silk particle described herein, wherein the silk-based material is permeable to said at least one active agent such that the active agent can be released through the silk-based material into an ambient surrounding at a pre-determined rate. In some embodiments, the pre-determined rate of the release can be controlled by, for example, adjusting an amount of beta-sheet conformation of silk fibroin present in the silk-based material, porosity of the silk-based material, or a combination thereof.

The composition can be maintained at any environmental condition. For example, in some embodiments, the composition can be maintained at about room temperature. In other embodiments, the composition can be maintained at a temperature of about 37° C. or greater. In some embodiments, the composition can be maintained under exposure to light. In some embodiments, the composition can be maintained at a relative humidity of at least about 10% or higher.

In another aspect, provided herein is a method of delivering an active agent (e.g., a volatile, hydrophobic and/or lipophilic agent) comprising applying or administering to a subject at least one composition (including a storage-stable composition described herein) or at least one silk particle described herein, said silk-based material of the composition or silk particle being permeable to the active agent such that the active agent can be released through the silk-based material, at a pre-determined rate, upon application or administration of the composition to the subject.

Depending on purposes of the applications and/or application sites, in some embodiments, the active agent present in the second immiscible phase of the composition (e.g., a volatile, hydrophobic and/or lipophilic agent present in an interior oil phase) can be released to an ambient surrounding, e.g., air. In these embodiments, the composition can be applied to the subject topically. In one embodiment, the composition can be applied on a skin or surface of a subject. The subject can be a living subject, e.g., a mammalian subject, or it can be a physical object, such as an article of manufacture.

Alternatively, the active agent present in the second immiscible phase of the composition (e.g., a volatile, hydrophobic and/or lipophilic agent present in an interior oil phase) can be released to a target biological cell of a subject when the composition is applied or administered in vivo. In these embodiments, the composition can be applied or administered to the subject orally or parenterally.

### BRIEF DESCRIPTION OF THE DRAWINGS

FIG. 1 is a schematic representation of an exemplary oil-encapsulated silk microparticle preparation using oil/water/oil (O/W/O) emulsions containing sonicated aqueous silk fibroin solution as the encapsulating water phase. Once sonicated, silk begins transitioning to the physically crosslinked water-insoluble hydrogel state, but remains in solution state for controllable durations dependent on, for example, the silk properties and/or sonication parameters. In the solution state, oil can be emulsified in the silk solution, and the W/O emulsion can be further emulsified in a continuous oil phase. In the continuous oil phase, the oil-encapsulated silk droplets are held in a spherical conformation until crosslinking completes, at which point the silk becomes a stable, water-insoluble hydrogel encapsulation matrix for the oil.

FIGS. 2A-2B are images showing emulsions of oil containing a dye mixed with an aqueous silk solution. FIG. 2A is an image showing an emulsion of sunflower oil containing Oil Red O mixed with a ˜7% (w/v) aqueous silk solution in a ˜1:3 (v/v) ratio of oil: silk, mixed with inversion (˜10 min) prior to sonication. FIG. 2B is an image showing an emulsion of sunflower oil containing Oil Red O mixed with a ˜7% (w/v) aqueous silk solution in a ˜1:3 (v/v) ratio of oil: silk, mixed with inversion (˜10 min) after gentle sonication (˜10% amplitude for ˜5 seconds). Scale bars=250 μm.

FIGS. 3A and 3B are images, respectively, showing hologram-patterned silk films prepared from (FIG. 3A) silk solution alone and (FIG. 3B) oil microemulsion (˜1:20 oil in silk; silk is ˜3% (w/v) prepared with a 45 minute degumming time) and cast using the same hologram-patterned mold.

FIGS. 4A-4F are photographs showing silk droplets in accordance with one or more embodiments described herein. FIG. 4A shows sonicated silk solution held in spherical droplets in a sunflower oil bath (silk has not completed transition to hydrogel state, as evidenced by the slight translucence of the particles). FIG. 4B shows sonicated silk solution containing a dispersion of Oil Red O loaded oil microdroplets held in spherical droplets in a sunflower oil bath. FIG. 4C is a side view of sonicated silk solution held in spherical droplets, wherein the sonicated silk solution contains green food coloring for ease of visualization. FIG. 4D shows that hydrogel silk spheres prepared from sonicated silk alone, allowed to complete crosslinking in a sunflower oil bath, retain their shape after removal from the oil bath. FIG. 4E shows that oil loaded silk hydrogel microspheres prior to dehydration (silk matrix is soft hydrogel). FIG. 4F shows that oil loaded silk spheres characterized by a firmer, denser silk encapsulation matrix resulting from dehydration of the silk hydrogel network with overnight drying at ambient conditions.

FIGS. 5A-5D are images showing active-agent loaded silk particles. FIG. 5A is a photograph showing silk hydrogel macroparticles loaded with doxorubicin prepared by pipetting controlled volumes of a sol-gel silk solution containing doxorubicin into a sunflower oil bath. FIG. 5B is a photograph showing silk hydrogel macroparticles loaded with a food coloring prepared by pipetting controlled volumes of a sol-gel silk solution containing food coloring into a sunflower oil bath and dehydrated silk macroparticles prepared by drying silk hydrogel macroparticles. FIGS. 5C-5D are images of silk microspheres prepared by sonication of silk into a sunflower oil bath (water/oil (W/O) emulsion) (silk contains 1:100 volumetric ratio a food coloring for visualization). Scale bar=100 μL.

FIGS. 6A-6B are images showing oil-encapsulated silk microparticles prepared using O/W/O emulsions, for example, with ˜60 minute degumming time regenerated silk fibroin solution. FIG. 6A is an image showing an O/W/O emulsion prepared with a ˜6% (w/v) silk solution sonicated at an amplitude of ˜15% for ˜45 seconds, wherein the silk was degummed for about ˜60 minutes. FIG. 6B is an image showing an O/W/O emulsion prepared with ˜3% (w/v) sonicated at an amplitude of ˜15% for ˜30 seconds, wherein the silk was degummed for about 60 minutes. Scale bars=300 μm.

FIGS. 7A-7D are images showing oil-encapsulated silk microparticles prepared using O/W/O emulsions with a ˜6% (w/v) silk solution treated with different sonication parameters, wherein the silk was degummed for ˜30 minutes. FIGS. 7A-7B show oil-encapsulated silk microparticles where silk was sonicated at an amplitude of ˜10% for ˜15 seconds. FIGS. 7C-7D show oil-encapsulated silk microparticles where silk was sonicated at an amplitude of ˜15% for ˜15 seconds.

FIGS. 8A-8D are absorbance measurements (at ˜518 nm) of relative diffusion of oil (e.g., Oil Red O) from the internal oil capsule of silk microparticles to an external oil phase (e.g., a sunflower oil bath). FIG. 8A shows absorbance measurements corresponding to no sonication of silk. FIG. 8B shows absorbance measurements corresponding to a ˜3% (w/v) silk solution sonicated at ˜15% amplitude for about 30 seconds, with varying degumming duration of the silk (e.g., 30 minutes or 60 minutes). FIG. 8C shows absorbance measurements corresponding to a ˜6% (w/v) silk solution prepared using a ˜30 minute degumming duration followed by exposure to varied sonication: no sonication, sonication at ˜10% amplitude for ˜15 seconds, or sonication at ˜15% amplitude for ˜15 seconds. FIG. 8D shows absorbance measurements corresponding to a 6% (w/v) silk solution prepared using a ˜60 minute degumming duration followed by exposure to varied sonication: no sonication, sonication at ˜15% amplitude for ˜30 seconds, or sonication at ˜15% amplitude for ˜45 seconds.

FIGS. 9A-9B are images showing formation of a silk “skin” in O/W/O microspheres: at the exterior oil-water interface the silk skin appears “baggy” (FIG. 9A) or forms “wrinkles” (FIG. 9B, white arrows).

FIG. 10 is a set of photographs showing a time-course study of untreated, dye-loaded silk film dissolution in water. Untreated silk films loaded with indigo carmine (top row) and fluorescein (bottom row) begin dissolving within ˜3 minutes of exposure to ˜37° C. water and are fully dissolved after about 30 minutes of immersion.

FIGS. 11A-11B is a set of photographs showing free-standing 2D micro-prism arrays prepared by casting oil-silk microemulsion on reflector-patterned silicone molds. FIG. 11A is a photograph taken without flash and FIG. 11B was taken with flash, demonstrating retention of reflector functionality.

FIG. 12 is a photograph showing silk hydrogel spheres prepared by sonicating the silk solution, and adding food coloring to the sonicated silk while still in the solution state (volume of food coloring added held constant, ratio of red, blue and yellow food coloring varied as noted), aliquoting into oil bath and allowing crosslinking to complete at ambient conditions of pressure and temperature.

FIG. 13 shows that oil-water interface increases silk protein assembly around oil particles, as evidenced by decreased silk gelation time with addition of a sunflower oil layer.

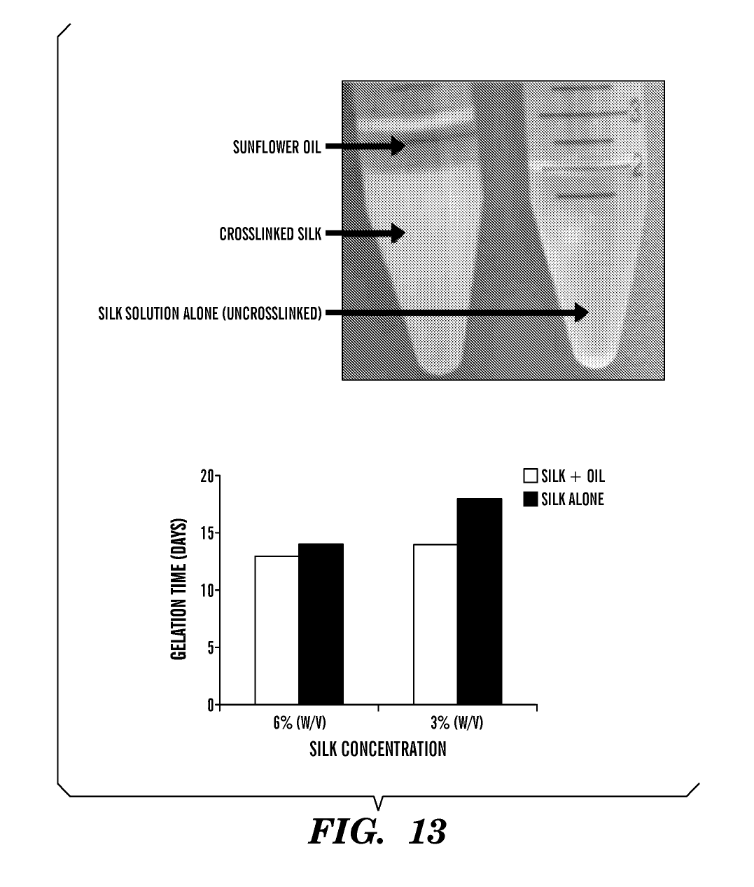

FIG. 14 is a set of images showing images of oil-encapsulated silk microparticles with different ratios of oil to silk. The images show that increasing the ratio of oil to silk can increase particle size.

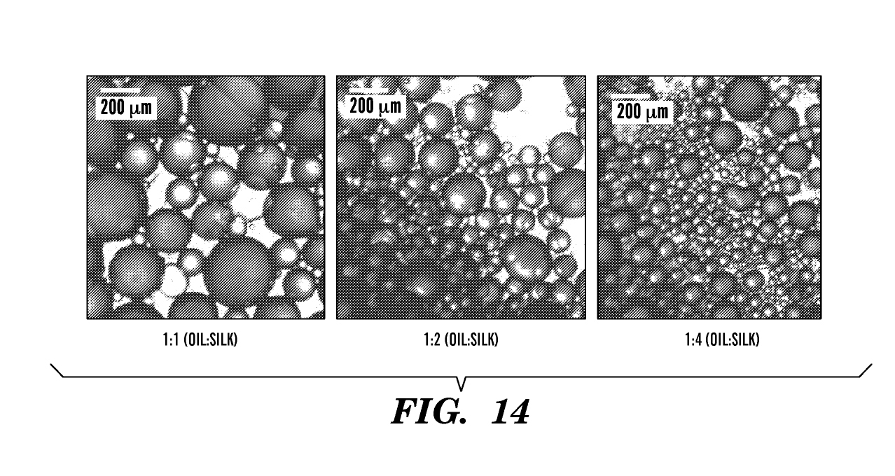

### DETAILED DESCRIPTION OF THE INVENTION

There is still an unmet need for development of novel encapsulation techniques that can improve the encapsulation efficiency of labile molecules (e.g., volatile, hydrophobic, and/or lipophilic molecules), protect and stabilize these labile molecules, and/or controllably release these labile molecules. The inventors have inter alia demonstrated novel techniques for encapsulating oil in silk biomaterials that can employ aqueous and ambient processing and tunable silk gelation behavior. For example, in some embodiments, gelation of silk fibroin can be induced by sonication, as described in U.S. Pat. No. 8,187,616, the process of which can provide a silk solution in a sol-gel state that can remain in the solution state long enough to perform a double emulsion before it gels. Thus, in some embodiments, stable silk micro- and macro-particles loaded with oil (optionally containing oil-soluble active agent(s)) or loaded with water-soluble active agent(s) can be produced, for example, by sonicating a silk solution (optionally comprising oil droplets) and aliquoting the sonicated silk solution into an oil bath. It was discovered that oil microdroplets were stably emulsified in aqueous silk solutions without addition of any emulsifiers and the presence of oil microdroplets did not impede self-assembly of silk into solid-state silk materials such as films or hydrogel networks. The inventors have further demonstrated that, in O/W/O emulsions, particle morphology and the permeability of the silk to a lipophilic active agent in the interior oil phase (or release of the lipophilic active agent from the interior oil phase to a surrounding) can be determined at least in part by silk solution concentration, silk processing and/or sonication. These stable emulsions of oil phase in silk biomaterials (oil-encapsulated silk biomaterials) can be used in various applications, e.g., in tissue engineering such as to model a tissue with a high lipid content, as well as for delivery and/or stabilization/storage of an active agent that are soluble in the oil phase of the silk biomaterials such as therapeutic agents, diagnostic agent, food additives such as food dyes or flavors, lipids, and cosmetically active agents or additives such as antioxidants, and volatile substances such as odor-releasing substances (e.g., fragrance or scents). Accordingly, embodiments of various aspects provided herein relate to compositions comprising an emulsion of an immiscible phase (e.g., a liquid oil phase) dispersed in a silk-based material, as well as methods of making and uses of the compositions.

### Silk-Based Compositions (e.g., Silk Particles) Comprising at Least Two Immiscible Phases

In one aspect, provided herein relates to silk-based emulsion compositions. The composition comprises at least two immiscible phases, a first immiscible phase comprising a silk-based material and a second immiscible phase comprising an active agent, wherein the first immiscible phase encapsulates the second immiscible phase. Stated another way, the second immiscible phase is dispersed in the first immiscible phase, forming an emulsion of the second immiscible phases dispersed in the first immiscible phase.

The term “immiscible” is used in its conventional sense to refer to two materials that are less than completely miscible, in that mixing two such materials results in a mixture containing more than one phase. In some embodiments, two immiscible phases as provided herein can be two fluids that are less than completely miscible. In some embodiments, two immiscible phases as provided herein can be a fluid and a solid material that form a solid-fluid interface. In some embodiments, two immiscible phases as provided herein can be two solids forming a solid-solid interface. In some embodiments, two “immiscible” phases as provided herein are completely or almost completely immiscible, i.e., give rise to a mixture containing two phases, wherein each phase contains at least about 95%, preferably at least about 99%, of a single phase. In addition, the term is intended to encompass situations wherein two immiscible phases can form an emulsion. For example, in one embodiment, the two immiscible phases can include silk-based material and lipid-based material, which can form an emulsion in which lipid droplets are dispensed in a silk-based material.

Second Immiscible Phase:

The second immiscible phase can be any fluid or material that can form an interface with the first immiscible phase comprising a silk-based material. Examples of the second immiscible phase include, but not limited to non-polar organic solvents, lipid components, polymers (e.g., but not limited to polyvinyl alcohol, poly(ethylene glycol), and block copolymers based on ethylene oxide and propylene oxide (e.g., PLURONIC®)), and hydrogels. In some embodiments, the second immiscible phase can comprise a lipid component, e.g., but not limited to, oil, fatty acids, glycerolipids, glycerophospholipids, sphingolipids, saccharolipids, polyketides, sterol lipids and prenol lipids.

In some embodiments, the second immiscible phase excludes a liposome. As used herein, the term “liposome” refers to a microscopic vesicle comprising one or more lipid bilayer(s). Structurally, liposomes range in size and shape from long tubes to spheres. Accordingly, in some embodiments, the lipid component excludes long-chain molecules comprising fatty acids that can form liposomes under suitable liposome forming conditions. Examples of such lipid component include, but are not limited to, phosphatidylcholine (PC), phosphatidylethanolamine (PE), phosphatidic acid (PA), phosphatidylglycerol (PG), sterol such as cholesterol, and normatural lipid(s), cationic lipid(s) such as DOTMA (N-(1-(2,3-dioxyloxyl)propyl)-N,N,N-trimethyl ammonium chloride), as well as 1,2-dioleoyl-sn-glycero-3-phosphocholine (DOPC); 1,2-dioleoyl-sn-glycero-3-phophoethanolamine (DOPE); 1,2-dilauroyl-sn-glycero-3-phosphocholine (DLPC); and 1,2-dimyristoyl-sn-glycero-3-phosphocholine (DMPC); and any combinations thereof. In some embodiments, the lipid component can exclude phospholipids. In some embodiments, the lipid component can exclude glycerophospholipids.

In some embodiments, the lipid component is oil. As used herein, the term “oil” refers in general to flowable (at room temperature) oils that are derived from natural sources such as animals or plants or are artificially made. In some embodiments, the term “oil” refers to flowable edible oils derived from animals or plants, including but not limited to fish oils, liquefied animal fats, and vegetable or plant oils, including but not limited to corn oil, coconut oil, soybean oil, olive oil, cottonseed oil, safflower oil, sunflower oil, canola, peanut oil, and combinations thereof (hydrogenated, non-hydrogenated, and partially hydrogenated oil). Additional examples of oils that can be used herein include, but are not limited to, plant oils (for example, Apricot Kernel Oil, Arachis Oil, Arnica Oil, Argan Oil, Avocado Oil, Babassu Oil, Baobab Oil, Black Seed Oil, Blackberry Seed Oil, Blackcurrant Seed Oil, Blueberry Seed Oil, Borage Oil, Calendula Oil, Camelina Oil, Camellia Seed Oil, Castor Oil, Cherry Kernel Oil, Cocoa Butter, Evening Primrose Oil, Grapefruit Oil, Grapeseed Oil, Hazelnut Oil, Hempseed Oil, Jojoba Oil, Lemon Seed Oil, Lime Seed Oil, Linseed Oil, Kukui Nut Oil, Macadamia Oil, Maize Oil, Mango Butter, Meadowfoam Oil, Melon Seed Oil, Moringa Oil, Orange Seed Oil, Palm Oil, Papaya Seed Oil, Passion Seed Oil, Peach Kernel Oil, Plum Oil, Pomegranate Seed Oil, Poppy Seed Oil, Pumpkins Seed Oil, Rapeseed (or Canola) Oil, Red Raspberry Seed Oil, Rice Bran Oil, Rosehip Oil, Seabuckthorn Oil, Sesame Oil, Strawberry Seed Oil, Sweet Almond Oil, Walnut Oil, Wheat Germ Oil); fish oils (for example: Sardine Oil, Mackerel Oil, Herring Oil, Cod-liver Oil, Oyster Oil); animal oils (for xxample: Conjugated Linoleic Acid); or other oils (for example: Paraffinic Oils, Naphthenic Oils, Aromatic Oils, Silicone Oils); or any mixture thereof.

The oil can comprise a liquid, or a combination of liquid and solid particles (e.g., fat particles in a liquid base). In addition, the term “oil” can include fat substitutes, which can be used alternatively or in combination with animal and/or plant oils. A suitable fat substitute is sucrose polyester, such as is available from the Procter & Gamble Co. under the trade name OLEAN®. The following U.S. Patents disclose fat substitutes, and are incorporated herein by reference: U.S. Pat. No. 4,880,657 issued Nov. 14, 1989; U.S. Pat. No. 4,960,602 issued Oct. 2, 1990, U.S. Pat. No. 4,835,001 issued May 30, 1989; U.S. Pat. No. 5,422,131 issued Jan. 2, 1996. Other suitable fat substitutes include SALATRIM® brand product from Nabisco and various alkoxylated polyols such as those described in the following U S. Patents incorporated herein by reference—U.S. Pat. Nos. 4,983,329; 5,175,323; 5,288,884; 5,298,637, 5,362,894; 5,387,429; 5,446,843; 5,589,217, 5,597,605, 5,603,978; and 5,641,534.

The number of second immiscible phases can vary with different applications. For example, in some embodiments, the second immiscible phase can form a single compartment or droplet. In other embodiments, the second immiscible phase can form a plurality of (e.g., at least two or more, including, 2, 3, 4, 5, 6, 7, 8, 9, 10, 20, 30, 40 or more) compartments or droplets.

The size and/or shape of the compartments or droplets can vary with a number of factors including, e.g., silk particle size, silk solution concentration and/or silk processing. In some embodiments, the size of the compartments or droplets can be in a range of about 1 nm to about 1000 μm, or about 5 nm to about 500 μm. In some embodiments, the size of the compartments or droplets can be in range of about 1 nm to about 1000 nm, or about 2 nm to about 750 nm, or about 5 nm to about 500 nm, or about 10 nm to about 250 nm. In some embodiments, the size of the compartments or droplets can be in a range of about 1 μm to about 1000 μm, or about 5 μm to about 750 μm, or about 10 μm to about 500 μm, or about 25 μm to about 250 μm.

Any active agent that is preferentially soluble in the second immiscible phase (e.g., oil) and/or is desired to be dispersed in the second immiscible phase (e.g., oil) can be included in the second immiscible phase. As referred to herein the term “preferentially soluble” should be understood to refer to a higher level or rate of solubility of the active agent in the second immiscible phase than in the first immiscible phase (e.g., silk-based material), for example, by at least about 10% or more, including, e.g., at least about 20%, at least about 30%, at least about 40%, at least about 50%, at least about 60%, at least about 70%, at least about 80%, at least about 90%, at least about 95% or more. In some embodiments, the level or rate of solubility of the active agent in the second immiscible phase can be higher than in the first immiscible phase by at least about 1.5-fold, at least about 2-fold, at least about 3-fold, at least about 4-fold, at least about 5-fold, at least about 10-fold, or more. In some embodiments, the term “preferentially soluble” refers to an active agent completely insoluble in the first immiscible phase but is partially or completely soluble in the second immiscible phase.

In some embodiments, the active agent present in the second immiscible phase is a volatile, hydrophobic and/or lipophilic agent. Examples of the volatile, hydrophobic and/or lipophilic molecule include, without limitations, a therapeutic agent, a nutraceutical agent, a cosmetic agent, a food additive (e.g., a coloring agent or a flavoring substance), a probiotic agent, a dye, an aromatic compound, an odor-releasing substance, an aliphatic compound (e.g., but not limited to, alkane, alkene, alkyne, a cycloaliphatic compound such as cyclo-alkane, cyclo-alkene, and cyclo-alkyne), a small molecule, and any combinations thereof.

As used herein, the term “volatile agent” refers to a molecule, substance, composition or a component thereof that is vaporizable. The volatile agent includes, but is not limited to “odor-releasing substance.” As used herein, the term “odor-releasing substance” refers to a molecule, substance, composition, or a component thereof capable of imparting to an ambient surrounding an odor, including, but not limited to pleasant, and savory smells, and, thus, also encompass scents or odors that function as insecticides, insect repellants, air fresheners, deodorants, aromacology, aromatherapy, or any other odor that acts to condition, modify, or otherwise charge the atmosphere or to modify the environment. It should be understood that perfumes, fragrance, aromatic materials, and/or scents, e.g., used in fragrance preparations, foods, cosmetics, personal care products, etc., generally comprise one or more volatile agents and are thus encompassed herein. In some embodiments, a volatile agent can include natural perfumes extracted from natural matter, such as fruits, plants, flowers, e.g., rose essential oil and peppermint essential oil, and synthetic perfumes artificially prepared, such as limonene and linalool. Aromatic plant parts, such as fruits, herbs, and trees, (including dried plant parts such as potpourri) can also be encompassed herein.

In some embodiments, the volatile agent can be a volatile oil. The term “volatile oil” means an oil (or a non-aqueous medium) that can evaporate on contact with the skin in less than one hour at room temperature and atmospheric pressure. In some embodiments, the volatile oil can be a volatile cosmetic oil, which is liquid at room temperature, e.g., having a non-zero vapor pressure, at room temperature and atmospheric pressure, for example, having a vapor pressure ranging from 0.13 Pa to 40,000 Pa (10⁻³ to 300 mmHg), from 1.3 Pa to 13,000 Pa (0.01 to 100 mmHg) or from 1.3 Pa to 1300 Pa (0.01 to 10 mmHg).

As used herein, the term “hydrophobic agent” refers to a molecule, substance, composition, or a component thereof having a greater solubility in non-aqueous medium (e.g., organic solvent or lipophilic solvent) than in an aqueous medium, e.g., by at least about 10% or more. In some embodiments, the hydrophobic agent can have a greater solubility in a non-aqueous medium (e.g., organic solvent or lipophilic solvent) than in an aqueous medium by at least about 10% or more, including, e.g., at least about 20%, at least about 30%, at least about 40%, at least about 50%, at least about 60%, at least about 70%, at least about 80%, at least about 90% or more. In some embodiments, the hydrophobic agent can have a greater solubility in a non-aqueous medium (e.g., organic solvent or lipophilic solvent) than in an aqueous medium by at least about 1.5-fold or more, including, e.g., at least about 2-fold, at least about 3-fold, at least about 4-fold, at least about 5-fold, at least about 6-fold, at least about 7-fold, at least about 8-fold, at least about 9-fold or more.

As used herein, the term “lipophilic agent” refers to a molecule, substance, composition, or a component thereof having a greater solubility in oils, fats, lipids, and/or non-polar solvents such as hexane or toluene than in an aqueous medium, e.g., by at least about 10% or more. In some embodiments, the lipophilic agent can have a greater solubility in a oils, fats, lipids, and/or non-polar solvents than in an aqueous medium by at least about 10% or more, including, e.g., at least about 20%, at least about 30%, at least about 40%, at least about 50%, at least about 60%, at least about 70%, at least about 80%, at least about 90% or more. In some embodiments, the lipophilic agent can have a greater solubility in a oils, fats, lipids, and/or non-polar solvents than in an aqueous medium by at least about 1.5-fold or more, including, e.g., at least about 2-fold, at least about 3-fold, at least about 4-fold, at least about 5-fold, at least about 6-fold, at least about 7-fold, at least about 8-fold, at least about 9-fold or more.

In some embodiments, the second immiscible phase can further encapsulate a third immiscible phase. In some embodiments, the third immiscible phase can comprise an aqueous phase. For example, the third immiscible phase can comprise a silk-based material. Alternatively or additionally, the third immiscible phase can comprise a material that is partially or completely immiscible with the second immiscible phase, e.g., a hydrogel material.

The volumetric ratio of the combined second immiscible phase (e.g., lipid compartment(s) or droplet(s)) to the first immiscible phase (e.g., a silk-based material) can vary with the emulsion configuration (e.g., “microsphere” vs. “microcapsule” as described herein), silk solution concentration, silk processing, sonication treatment, and/or applications of the composition. In some embodiments, the volumetric ratio of the lipid compartment(s) or droplet(s) to the silk-based material can range from about 1000:1 to about 1:1000, from about 500:1 to about 1:500, from about 100:1 to about 1:100, or form about 10:1 to about 1:10. In some embodiments, the volumetric ratio of the lipid compartment(s) or droplet(s) to the silk-based material can range from about 1:1 to about 1:1000, from about 1:2 to about 1:500, or from about 1:5 to about 1:100, or from about 1:10 to about 1:100. In one embodiments, the volumetric ratio of the lipid compartment(s) or droplet(s) to the silk-based material can range from about 1:5 to about 1:20.

First Immiscible Phase:

The first immiscible phase comprises a silk-based material. As used herein, the term “silk-based material” refers to a material in which silk fibroin constitutes at least about 10% of the total material, including at least about 20%, at least about 30%, at least about 40%, at least about 50%, at least about 60%, at least about 70%, at least about 80%, at least about 90%, at least about 95%, up to and including 100% or any percentages between about 30% and about 100%, of the total material. In certain embodiments, the silk-based material can be substantially formed from silk fibroin. In various embodiments, the silk-based material can be substantially formed from silk fibroin and at least one active agent. In some embodiments where the silk fibroin constitute less than 100% of the total material, the silk-based material can comprise an additive, e.g., a different material and/or component including, but not limited to, a metal, a synthetic polymer, e.g., but not limited to, poly(vinyl alcohol) and poly(vinyl pyrrolidone), a hydrogel, nylon, an electronic component, an optical component, an active agent, any additive described herein, and any combinations thereof.

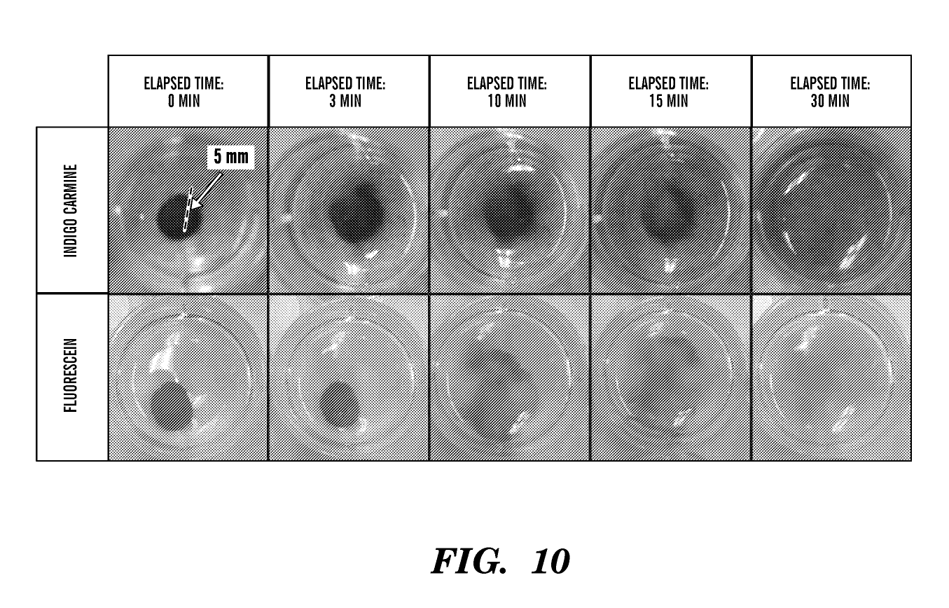

The solubility of the silk-based material can be adjusted, e.g., based on beta sheet content. Accordingly, in some embodiments, at least the silk-based material in the first immiscible phase can be soluble or redissolved in an aqueous solution. Hence, in some embodiments, the silk-based emulsion composition described herein can be dissolvable. For example, the dissolvable silk-based emulsion composition (e.g., in a form of a film) can dissolve upon exposure to an aqueous environment such as immersion in buffer (FIG. 10) or when brought into contact with a moist or hydrated tissue or surface. Dissolution of the silk-based material that encapsulates lipid droplets (e.g., oil droplets comprising an active agent) can result in release of the lipid droplets and thus the active agent loaded therein, if any, to the surrounding environment.

In alternative embodiments, at least the silk-based material in the first immiscible phase can be insoluble in an aqueous solution. For example, the beta-sheet content in silk fibroin can be increased by exposing the silk-based material to a post-treatment that increases beta-sheet formation to an amount sufficient to enable a silk-based material to resist dissolution in an aqueous medium.

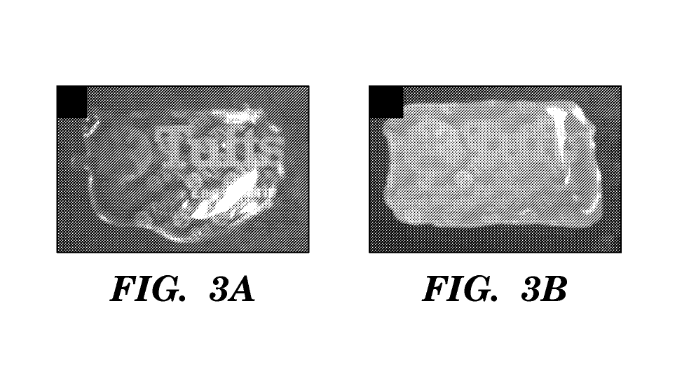

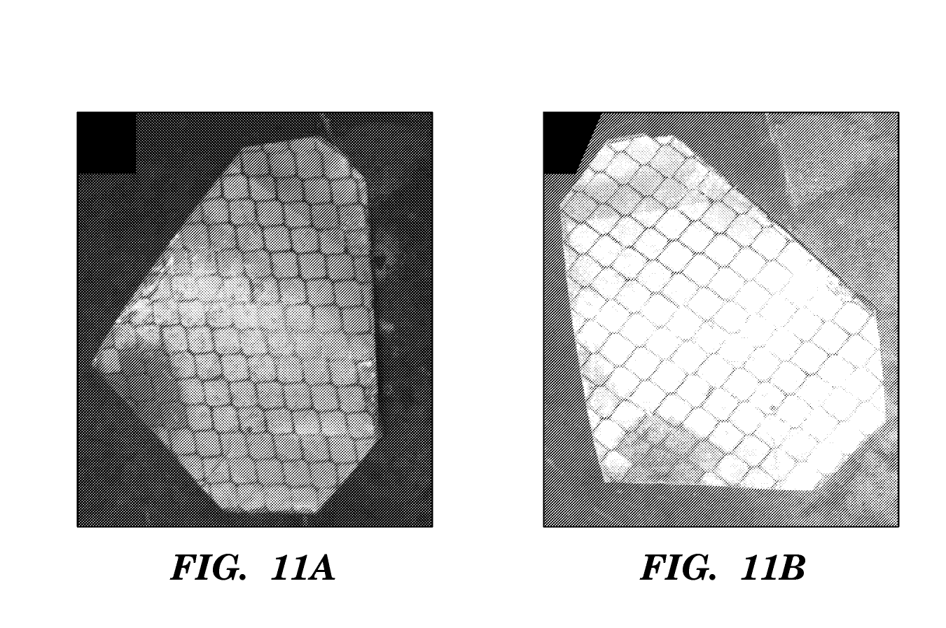

In some embodiments, the silk-based material can further comprise an optical or photonic pattern on at least one of its surface. For example, the optical or photonic pattern can comprise patterned diffractive optical surfaces such as holographic diffraction gratings and/or an array of patterns that provides an optical functionality, e.g., but not limited to, light reflection, diffraction, scattering, iridescence, and any combinations thereof. Methods for forming an optical or photonic pattern on a silk-based material are described here International Patent Appl. Nos. WO 2009/061823 and WO 2009/155397, the contents of which are incorporated herein by reference. For example, as shown in Example 2, an oil-silk microemulsion can be casted on a hologram mold, a plastic sheeting with an iridescent surface, or a reflector-patterned silicone mold, and the resulting silk-based emulsion composition can retain the optical property (e.g., holographic diffraction, iridescence, and/or light reflection) as shown in FIGS. 3A-3B and FIGS. 11A-11B.

In some embodiments, the first immiscible phase can further comprise one or more (e.g., one, two, three, four, five or more) active agents. In some embodiments, the active agent(s) can be incorporated into the silk-based material. The active agent(s) can be covalently or non-covalently linked with silk fibroin and/or can be integrated homogenously or heterogeneously within the silk fibroin-based material. Total amount of the active agent(s) in the first immiscible phase and/or the silk-based material can be in a range of about 0.1 wt % to about 0.99 wt %, about 0.1 wt % to about 70 wt %, about 5 wt % to about 60 wt %, about 10 wt % to about 50 wt %, about 15 wt % to about 45 wt %, or about 20 wt % to about 40 wt %, of the total silk fibroin in the composition. In some embodiments, the active agent(s) incorporated into the first immiscible phase can be an active agent that is water soluble and/or is able to be dispersed in the first immiscible phase.

Additive:

In some embodiments, the first immiscible phase can further comprise one or more (e.g., one, two, three, four, five or more) additives. In some embodiments, the additive(s) can be incorporated into the silk-based material. The additive can be covalently or non-covalently linked with silk fibroin and/or can be integrated homogenously or heterogeneously within the silk fibroin-based material. Without wishing to be bound by theory, an additive can provide one or more desirable properties to the composition or solid-state silk fibroin or silk fibroin article, e.g., strength, flexibility, ease of processing and handling, biocompatibility, solubility, bioresorbability, lack of air bubbles, surface morphology, release rate and/or enhanced stability of an active agent, if any, encapsulated therein, optical function, therapeutic potential, and the like.

An additive can be selected from biocompatible polymers or biopolymers; plasticizers (e.g., glycerol); emulsion stabilizers (e.g., lecithin, and polyvinyl alcohol), surfactants (e.g., polysorbate-20); interfacial tension-modulating agents such as surfactants (e.g., salt); beta-sheet inducing agents (e.g., salt); detectable agents (e.g., a fluorescent molecule); small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof. Furthermore, the additive can be in any physical form. For example, the additive can be in the form of a particle, a fiber, a film, a tube, a gel, a mesh, a mat, a non-woven mat, a powder, a liquid, or any combinations thereof. In some embodiments, the additive can be a particle (e.g., a microparticle or nanoparticle). In some embodiments, the additive can comprise a second active agent. The second active agent can be any active agent that is preferentially soluble in the first immiscible phase and/or is desired to be dispersed in the first immiscible phase.

Total amount of additives in the first immiscible phase and/or the silk-based material can be in a range of about 0.1 wt % to about 0.99 wt %, about 0.1 wt % to about 70 wt %, about 5 wt % to about 60 wt %, about 10 wt % to about 50 wt %, about 15 wt % to about 45 wt %, or about 20 wt % to about 40 wt %, of the total silk fibroin in the composition.

Appropriate additive(s) can be selected to add into the first immiscible phase and/or the silk based material, e.g., depending on various applications. By way of example only, when the composition described herein is a tissue scaffold comprising a plurality of second immiscible phases (e.g., lipid compartments or droplets) dispersed in a silk-based material, the composition can comprise an additive that increases the mechanical performance of the scaffold, if needed, and/or improve cell behavior (e.g., proliferation, adhesion, and/or viability) within the scaffold.

By way of example only, in some embodiments, the additive can include a calcium phosphate (CaP) material. As used herein, the term “calcium phosphate material” refers to any material composed of calcium and phosphate ions. The term “calcium phosphate material” is intended to include naturally occurring and synthetic materials composed of calcium and phosphate ions. The calcium phosphate material can be selected, for example, from one or more of brushite, octacalcium phosphate, tricalcium phosphate (also referred to as tricalcic phosphate and calcium orthophosphate), calcium hydrogen phosphate, calcium dihydrogen phosphate, apatite, and/or hydroxyapatite. Further, tricalcium phosphate (TCP) can be in the alpha or the beta crystal form. In some embodiments, the calcium phosphate material is beta-tricalcium phosphate or apatite, e.g., hydroxyapatite (HA).

In some embodiments, the first immiscible phase and/or the silk-based material can comprise magnetic particles to form magneto-sensitive compositions as described in International Patent Application No. PCT/US13/36539 filed Apr. 15, 2013, the content of which is incorporated herein by reference.

In some embodiments, the first immiscible phase and/or the silk-based material can comprise a silk material as an additive, for example, to produce a silk fibroin composite (e.g., 100% silk composite in the first immiscible phase) with improved mechanical properties. Examples of silk materials that can be used as an additive include, without limitations, silk particles, silk fibers, silk micron-sized fibers, silk powder and unprocessed silk fibers. In some embodiments, the additive can be a silk particle or powder. Various methods of producing silk fibroin particles (e.g., nanoparticles and microparticles) are known in the art. In some embodiments, the silk particles can be produced by a polyvinyl alcohol (PVA) phase separation method as described in, e.g., International App. No. WO 2011/041395, the content of which is incorporated herein by reference in its entirety. Other methods for producing silk fibroin particles are described, for example, in U.S. App. Pub. No. U.S. 2010/0028451 and PCT App. Pub. No.: WO 2008/118133 (using lipid as a template for making silk microspheres or nanospheres), and in Wenk et al. J Control Release, Silk fibroin spheres as a platform for controlled drug delivery, 2008; 132:26-34 (using spraying method to produce silk microspheres or nanospheres), content of all of which is incorporated herein by reference in its entirety.

Generally, silk fibroin particles or powder can be obtained by inducing gelation in a silk fibroin solution and reducing the resulting silk fibroin gel into particles, e.g., by grinding, cutting, crushing, sieving, sifting, and/or filtering. Silk fibroin gels can be produced by sonicating a silk fibroin solution; applying a shear stress to the silk solution; modulating the salt content of the silk solution; and/or modulating the pH of the silk solution. The pH of the silk fibroin solution can be altered by subjecting the silk solution to an electric field and/or reducing the pH of the silk solution with an acid. Methods for producing silk gels using sonication are described for example in U.S. Pat. App. Pub No. U.S. 2010/0178304 and Int. Pat. App. Pub. No. WO 2008/150861, contents of both which are incorporated herein by reference in their entirety. Methods for producing silk fibroin gels using shear stress are described, for example, in International Patent App. Pub. No.: WO 2011/005381, the content of which is incorporated herein by reference in its entirety. Methods for producing silk fibroin gels by modulating the pH of the silk solution are described, for example, in U.S. Pat. App. Pub. No.: US 2011/0171239, the content of which is incorporated herein by reference in its entirety.

In some embodiments, silk particles can be produced using a freeze-drying method as described in U.S. Provisional Application Ser. No. 61/719,146, filed Oct. 26, 2012; and International Pat. App. No. PCT/US13/36356 filed: Apr. 12, 2013, content of each of which is incorporated herein by reference in its entirety. Specifically, a silk fibroin foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure.

Optionally, the conformation of the silk fibroin in the silk fibroin foam can be altered after formation. Without wishing to be bound by theory, the induced conformational change can alter the crystallinity of the silk fibroin in the silk particles, e.g., silk II beta-sheet crystallinity. This can alter the rate of release of an active agent from the silk matrix. The conformational change can be induced by any methods known in the art, including, but not limited to, alcohol immersion (e.g., ethanol, methanol), water annealing, water vapor annealing, heat annealing, shear stress (e.g., by vortexing), ultrasound (e.g., by sonication), pH reduction (e.g., pH titration), and/or exposing the silk particles to an electric field and any combinations thereof.

In some embodiments, no conformational change in the silk fibroin is induced, i.e., crystallinity of the silk fibroin in the silk fibroin foam is not altered or changed before subjecting the foam to particle formation.

After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

Without limitations, the silk fibroin particles can be of any desired size. In some embodiments, the particles can have a size ranging from about 0.01 μm to about 1000 μm, about 0.05 μm to about 500 μm, about 0.1 μm to about 250 μm, about 0.25 μm to about 200 μm, or about 0.5 μm to about 100 μm. Further, the silk particle can be of any shape or form, e.g., spherical, rod, elliptical, cylindrical, capsule, or disc.

In some embodiments, the silk fibroin particle can be a microparticle or a nanoparticle. In some embodiments, the silk particle can have a particle size of about 0.01 μm to about 1000 μm, about 0.05 μm to about 750 μm, about 0.1 μm to about 500 μm, about 0.25 μm to about 250 μm, or about 0.5 μm to about 100 μm. In some embodiments, the silk particle has a particle size of about 0.1 nm to about 1000 nm, about 0.5 nm to about 500 nm, about 1 nm to about 250 nm, about 10 nm to about 150 nm, or about 15 nm to about 100 nm.

The amount of the silk fibroin particles in the first immiscible phase and/or the silk-based material can range from about 1% to about 99% (w/w or w/v). In some embodiments, the amount the silk particles in the first immiscible phase and/or the silk-based material can be from about 5% to about 95% (w/w or w/v), from about 10% to about 90% (w/w or w/v), from about 15% to about 80% (w/w or w/v), from about 20% to about 75% (w/w or w/v), from about 25% to about 60% (w/w or w/v), or from about 30% to about 50% (w/w or w/v).). In some embodiments, the amount of the silk particles in the first immiscible phase and/or the silk-based material can be less than 20%.

Generally, the composition described herein can comprise any ratio of silk fibroin to silk fibroin particles. For example, the ratio of silk fibroin to silk particles in the solution can range from about 1000:1 to about 1:1000. The ratio can be based on weight or moles. In some embodiments, the ratio of silk fibroin to silk particles in the solution can range from about 500:1 to about 1:500 (w/w), from about 250:1 to about 1:250 (w/w), from about 50:1 to about 1:200 (w/w), from about 10:1 to about 1:150 (w/w) or from about 5:1 to about 1:100 (w/w). In some embodiments, ratio of silk fibroin to silk particles in the solution can be about 1:99 (w/w), about 1:4 (w/w), about 2:3 (w/w), about 1:1 (w/w) or about 4:1 (w/w). In some embodiments, the amount of silk particles is equal to or less than the amount of the silk fibroin, i.e., a silk fibroin to silk particle ratio of 1:1. In some embodiments, the ratio of high molecular weight silk fibroin to silk particles in the composition can be about 1:1, about 1:0.75, about 1:0.5, or about 1:0.25.

In some embodiments, the additive can be a silk fiber. In some embodiments, silk fibers can be chemically attached by redissolving part of the fiber in HFIP and attaching to the first immiscible phase and/or the silk-based material, for example, as described in US patent application publication no. US20110046686, the content of which is incorporated herein by reference.

In some embodiments, the silk fibers can be microfibers or nanofibers. In some embodiments, the additive can be micron-sized silk fiber (10-600 μm). Micron-sized silk fibers can be obtained by hydrolyzing the degummed silk fibroin or by increasing the boing time of the degumming process. Alkali hydrolysis of silk fibroin to obtain micron-sized silk fibers is described for example in Mandal et al., PNAS, 2012, doi: 10.1073/pnas.1119474109; and PCT application no. PCT/US13/35389, filed Apr. 5, 2013, content of all of which is incorporated herein by reference. Because regenerated silk fibers made from HFIP silk solutions are mechanically strong, in some embodiments, the regenerated silk fibers can also be used as an additive.

In some embodiments, the silk fiber can be an unprocessed silk fiber, e.g., raw silk or raw silk fiber. The term “raw silk” or “raw silk fiber” refers to silk fiber that has not been treated to remove sericin, and thus encompasses, for example, silk fibers taken directly from a cocoon. Thus, by unprocessed silk fiber is meant silk fibroin, obtained directly from the silk gland. When silk fibroin, obtained directly from the silk gland, is allowed to dry, the structure is referred to as silk I in the solid state. Thus, an unprocessed silk fiber comprises silk fibroin mostly in the silk I conformation. A regenerated or processed silk fiber on the other hand comprises silk fibroin having a substantial silk II or beta-sheet crystallinity.

In some embodiments, the additive can comprise at least one biocompatible polymer, including at least two biocompatible polymers, at least three biocompatible polymers or more. For example, the first immiscible phase and/or the silk-based material can comprise one or more biocompatible polymers in a total concentration of about 0.1 wt % to about 70 wt %, about 1 wt % to about 60 wt %, about 10 wt % to about 50 wt %, about 15 wt % to about 45 wt % or about 20 wt % to about 40 wt %. In some embodiments, the biocompatible polymer(s) can be incorporated homogenously or heterogeneously into the first immiscible phase and/or the silk-based material. In other embodiments, the biocompatible polymer(s) can be coated on a surface of the first immiscible phase and/or the silk-based material. In any embodiments, the biocompatible polymer(s) can be covalently or non-covalently linked to silk fibroin in the first immiscible phase and/or the silk-based material. In some embodiments, the biocompatible polymer(s) can be blended with silk fibroin within the first immiscible phase and/or the silk-based material.

Examples of the biocompatible polymers can include non-degradable and/or biodegradable polymers, e.g., but are not limited to, poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, alginate, chitosan, chitin, hyaluronic acid, pectin, polyhydroxyalkanoates, dextrans, and polyanhydrides, polyethylene oxide (PEO), poly(ethylene glycol) (PEG), triblock copolymers, polylysine, alginate, polyaspartic acid, any derivatives thereof and any combinations thereof. See, e.g., International Application Nos.: WO 04/062697; WO 05/012606. The contents of the international patent applications are all incorporated herein by reference. Other exemplary biocompatible polymers amenable to use according to the present disclosure include those described for example in U.S. Pat. No. 6,302,848; No. 6,395,734; No. 6,127,143; No. 5,263,992; No. 6,379,690; No. 5,015,476; No. 4,806,355; No. 6,372,244; No. 6,310,188; No. 5,093,489; No. US 387,413; No. 6,325,810; No. 6,337,198; No. U.S. Pat. No. 6,267,776; No. 5,576,881; No. 6,245,537; No. 5,902,800; and No. 5,270,419, content of all of which is incorporated herein by reference.

In some embodiments, the biocompatible polymer can comprise PEG or PEO. As used herein, the term “polyethylene glycol” or “PEG” means an ethylene glycol polymer that contains about 20 to about 2000000 linked monomers, typically about 50-1000 linked monomers, usually about 100-300. PEG is also known as polyethylene oxide (PEO) or polyoxyethylene (POE), depending on its molecular weight. Generally PEG, PEO, and POE are chemically synonymous, but PEG has previously tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g. viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete PEGs are also available with different geometries.

As used herein, the term PEG is intended to be inclusive and not exclusive. The term PEG includes poly(ethylene glycol) in any of its forms, including alkoxy PEG, difunctional PEG, multiarmed PEG, forked PEG, branched PEG, pendent PEG (i.e., PEG or related polymers having one or more functional groups pendent to the polymer backbone), or PEG With degradable linkages therein. Further, the PEG backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)m in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as biocompatible polymers.

Some exemplary PEGs include, but are not limited to, PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG15000, PEG 20000, PEG250000, PEG500000, PEG100000, PEG2000000 and the like. In some embodiments, PEG is of MW 10,000 Dalton. In some embodiments, PEG is of MW 100,000, i.e. PEO of MW 100,000.

In some embodiments, the additive can include an enzyme that hydrolyzes silk fibroin. Without wishing to be bound by theory, such enzymes can be used to control the degradation of the first immiscible phase and/or the silk-based material.

In some embodiments, the additive that can be included in the first immiscible phase and/or the silk-based material can include, but are not limited to, a biocompatible polymer described herein, an active agent described herein, a plasmonic particle, glycerol, and any combinations thereof.

In some embodiments, the silk-based material can be porous. For example, the porous silk-based material can be produced by subjecting the composition described herein to lyophilization. In these embodiments, the silk-based material can have a porosity of at least about 1%, at least about 5%, at least about 10%, at least about 20%, at least about 30%, at least about 40%, at least about 50%, at least about 60%, at least about 70%, at least about 80%, at least about 90%, or higher. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The porous silk-based material can have any pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pores of the solid-state silk fibroin can have a size distribution ranging from about 1 nm to about 1000 μm, from about 5 nm to about 500 μm, from about 10 nm to about 250 μm, from about 50 nm to about 200 μm, from about 100 nm to about 150 μm, or from about 1 μm to about 100 μm. In some embodiments, the silk-based material can be swellable when hydrated. The sizes of the pores can then change depending on the water content in the silk matrix. In some embodiment, the pores can be filled with a fluid such as water or air.

In some embodiments, the silk-based material can further comprise on at least a portion of its surface one or more coatings. The coating(s) can provide functional and/or physical property to the silk-based material (e.g., but not limited to controlling the release rate of an active agent encapsulated therein; maintaining hydration of the silk-based material; controlling the surface smoothness; and/or attaching a targeting ligand for targeted delivery).

Any biocompatible polymer described herein can be used for coating the outer surface of the silk particles described herein. In some embodiments, the coating can comprise a hydrophilic polymer. As used herein, the term “hydrophilic polymer” refers to a polymer that is water-soluble and/or capable of retaining water. Examples of hydrophilic polymer include, but are not limited to, homopolymers such as cellulose-base polymer, protein-based polymer, water-soluble vinyl-base polymer, water-soluble acrylic acid-base polymer and acrylamide-base polymer, and synthetic polymers such as crosslinked hydrophilic polymer. In some embodiments, a hydrophilic polymer for use in the coating can include one or any combinations of polyethylene glycol, polyethylene oxide, polyethylene glycol copolymers (e.g., poly(ethylene glycol-co-propylene glycol) copolymers, poly(ethylene glycol)-poly(propylene glycol)-poly(ethylene glycol) block copolymers, or poly(propylene glycol)-poly(ethylene glycol)-poly(propylene glycol) block copolymers), poly(propylene glycol), poly(2-hydroxyethyl methacrylate), poly(vinyl alcohol), poly(acrylic acid), poly(methacrylic acid), polyvinylpyrrolidone, cellulose ether, alginate, chitosan, hyaluronate, collagen, and mixtures or combinations thereof. In some embodiments, the coating can comprise polyethylene glycol and/or poly(ethylene oxide). In some embodiments, the coating can comprise a hydrophobic polymer (e.g., a polymer that is not hydrophilic as defined herein).

There can be any number of coatings, e.g., 1, 2, 3, 4, 5, 6, or more coatings, on the surface of the silk-based material. In some embodiments, there is at least 2, at least 3, at least 4, at least 5, at least 6 or more coatings.

Each coating can comprise at least one or more layers, for example, 1, 2, 3, 4, 5 layers. The material in each layer can be different or alternate. In one embodiment, a coating can have at least two layers.

In some embodiments, the coating can comprise a silk fibroin layer. See, e.g., International App. No. WO 2007/016524 for description of an example method to form silk coating. In some embodiments, the coating can comprise a biocompatible polymer layer (e.g., comprising a hydrophilic polymer as described herein) overlaid with a silk layer. In these embodiments, the hydrophilic polymer layer can comprise poly(ethylene oxide) (PEO).

In some embodiments, the coating can further comprise an additive as described herein. For example, the coating can further comprise a contrast agent and/or a dye.

The silk-based material can be present in any form or shape. Some forms of the silk-based material are described in the section “Examples of various forms of the silk-based material” below. For example, the silk-based material can be in a form of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized material, a porous material, a non-porous material, or any combinations thereof. In some embodiments, the silk-based material can be present in a hydrated state (e.g., as a hydrogel). In some embodiments, the silk-based material can be present in a dried state, e.g., by drying under an ambient condition and/or by lyophilization.

In some embodiments, the silk-based material can form a film. The second immiscible phases (e.g., oil droplets) can be uniformly or randomly dispersed in the silk-based film. In some embodiments, the presence of lipid droplets (e.g., oil droplets) in the silk-based films can render the film opaque rather than transparent as seen in a silk-based film alone (without emulsion of lipid droplets). Higher degree of opaqueness can result in a silk-based emulsion film when higher concentrations of lipid droplets (e.g., oil droplets) are present in the film.

In some embodiments, the silk-based material can form a scaffold. The second immiscible phases (e.g., oil droplets) can be dispersed uniformly or randomly in the silk-based scaffold.

A Silk Particle Loaded with One or More Lipid or Oil Droplets:

In some embodiments, the silk-based material can form a particle. In a particular aspect, provided herein is a silk particle comprising silk fibroin and at least one or more lipid droplets (e.g., oil droplets) encapsulated therein. The silk particle comprises at least two immiscible phases, a first immiscible phase comprising silk fibroin and a second immiscible phase comprising an active agent, wherein the first immiscible phase encapsulates the second immiscible phase (or stated another way, the second immiscible phase is dispersed in the first immiscible phase). In some embodiments, the second immiscible phases can exclude a liposome.

The size of the silk particle can vary based on the needs of various applications, e.g., cosmetics, therapeutics, and/or tissue engineering applications. Thus, the silk particle can be of any size. For example, the size of the silk particle can range from about 10 nm to about 10 mm, or from about 50 nm to about 5 mm. In some embodiments, the size of the silk particle can range from about 10 nm to about 1000 nm, or from about 10 nm to about 500 nm, or form about 20 nm to about 250 nm. In some embodiments, the size of the silk particle can range from about 1 μm to about 1000 μm, or from about 5 μm to about 500 μm, or form about 10 μm to about 250 μm. In some embodiments, the size of the silk particle can range from about 0.1 mm to about 10 mm, or from about 0.5 mm to about 10 mm, from about 0.5 mm to about 8 mm, or from about 1 mm to about 5 mm.

As noted above, the second immiscible phase can form a single or a plurality of (e.g., at least two or more) droplets of any size and/or shape in the silk particle. The size and/or shape of the droplets can vary with a number of factors including, e.g., silk solution concentration, silk processing, and/or size of the silk particle. In some embodiments, the size of the droplets can be in a range of about 1 nm to about 1000 μm, or about 5 nm to about 500 μm. In some embodiments, the size of the compartments or droplets can be in range of about 1 nm to about 1000 nm, or about 2 nm to about 750 nm, or about 5 nm to about 500 nm, or about 10 nm to about 250 nm. In some embodiments, the size of the compartments or droplets can be in a range of about 1 μm to about 1000 μm, or about 5 μm to about 750 μm, or about 10 μm to about 500 μm, or about 25 μm to about 250 μm.

The silk particle described herein can incorporate at least one or more of the features described for any embodiment of the silk-based emulsion compositions described above.

### Exemplary Compositions Comprising Silk Particles Described Herein

A further aspect provided herein is a composition comprising a collection or a plurality of silk particles described herein. The composition described herein can be used for any applications, e.g., but not limited to, personal care (including, e.g., skincare, hair care, cosmetics, and personal hygiene products), therapeutics, tissue engineering, and/or food products. Depending on intended uses, the compositions described herein can be formulated to form an emulsion, a colloid, a cream, a gel, a lotion, a paste, an ointment, a liniment, a balm, a liquid, a solid, a film, a sheet, a fabric, a mesh, a sponge, an aerosol, a powder, a scaffold, or any combinations thereof.

In some embodiments, the composition can be formulated for use in a pharmaceutical composition or product, e.g., a film, a tablet, a gel capsule, powder, an ointment, a liquid, a patch, or in a delivery device, e.g., a syringe. Additional description of pharmaceutical compositions comprising the silk particles described herein, e.g., for use in controlled or sustained release, is found in the section “Pharmaceutical compositions and controlled/sustained release” below.

In some embodiments, the composition can be formulated for use in a personal care composition. For example, in some embodiments, the personal care composition can be formulated to be a hair care composition or a skin care composition in a form of a cream, oil, lotion, powder, serum, gel, shampoo, conditioner, ointment, foam, spray, aerosol, mousse, or any combinations thereof. In other embodiments, the personal care composition can be formulated to be a cosmetic composition in a form of powder, lotion, cream, lipstick, nail varnish, hair dye, balm, spray, mascara, fragrance, solid perfume, or any combinations thereof.

In some embodiments, the personal care composition can comprise an odor-releasing composition (e.g., fragrance composition) in a form of a solid (e.g., wax), a film, a sheet, a fabric, a mesh, a sponge, powder, a liquid, a colloid, an emulsion, a cream, a gel, a lotion, a paste, an ointment, a liniment, a balm, a spray, a roll-on, or any combinations thereof. In some embodiments, the composition described herein can be used to stabilize and/or provide a controlled release or a sustained release of at least one odor-releasing substance, e.g., but not limited to fragrances, scents or any molecules/compositions that can impart a scent to the surrounding. For examples, at least one odor-releasing substance can be added to the first immiscible phase (e.g., the silk-based material) and/or the second immiscible phase (e.g., oil droplets), depending on their solubility in each phase. Generally, odor-releasing substances, e.g., but not limited to, fragrances and scents, can be oil-soluble. Accordingly, at least odor-releasing substance can be added to the second immiscible phase described herein (e.g., oil droplets). Additional information about personal care and fragrance compositions comprising the silk particles described herein is described in detail later in the sections “Personal care compositions” and “Odor-releasing compositions.”

In some embodiments, the composition can be formulated for use in a food composition, including, but not limited to, solid food, liquid food, drinks, emulsions, slurries, curds, dried food products, packaged food products, raw food, processed food, powder, granules, dietary supplements, edible substances/materials, chewing gums, or any combinations thereof. The food compositions can include, but are not limited to, food compositions consumed by any subject, including, e.g., a human, or a domestic or game animal such as feline species, e.g., cat; canine species, e.g., dog; fox; wolf; avian species, e.g., chicken, emu, ostrich, birds; and fish, e.g., trout, catfish, salmon and pet fish.

In some embodiments, the composition can be used to stabilize and/or provide a controlled release or a sustained release of at least one food ingredient, flavoring substance, nutrient, and/or vitamin. For examples, at least one food ingredient, flavoring substance, nutrient, and/or vitamins can be added to the first immiscible phase (e.g., the silk-based material) and/or the second immiscible phase (e.g., oil droplets), depending on their solubility in each phase. In some embodiments, the composition can be used as a food additive in the food composition. The food additive can be present in any form, e.g., powder, particles, slurry, liquid, solution, solid, emulsion, colloid or any combinations thereof. In some embodiments, the composition can be formulated for use as decoration in a food product, e.g., a decoration such as a hologram on a cake. In some embodiments, the composition described herein can be a “flavor composition” as described in the section below.

In accordance with various aspects described herein, silk can act as an emulsifier to stabilize an emulsion of lipid droplets dispersed in a silk-based material. Further, silk can stabilize or maintain activity of an active agent encapsulated therein as described in International Pat. App. No. WO 2012/145739, the content of which is incorporated herein by reference. Accordingly, a further aspect provided herein relates to a storage-stable silk-based emulsion composition. The storage-stable comprises a silk-based emulsion composition described herein or a silk particle described herein, wherein the active agent (e.g., a volatile, hydrophobic, and/or lipophilic agent) present in the second immiscible phase (e.g., lipid droplets) of the composition or the silk particle retains at least about 30% of its original bioactivity and/or original loading after the composition is (a) subjected to at least one freeze-thaw cycle, or (b) maintained for at least about 24 hours at about room temperature or above, or (c) both (a) and (b).

As used herein, the terms “maintaining,” “maintain,” and “maintenance,” when referring to active agents, mean keeping, sustaining, or retaining the bioactivity and/or loading of the agent when the agent is in a composition with silk fibroin. In some embodiments, the agent is maintained in the silk-based material of the composition described herein. In some embodiments, the agent is maintained in the interior lipid droplets (e.g., oil droplets) dispersed in the silk-based material of the composition described herein. In some embodiments, the active agent retains at least about 10% of its original bioactivity and/or original loading (e.g., 10%, 15%, 20%, 25%, 30%, 35%, 40%, 45%, 50%, 55%, 60%, 65%, 70%, 75%, 80%, 85%, 90%, 95% or more, of its original bioactivity and/or original loading).

As used herein, the term “bioactivity” generally refers to the ability of an active agent to interact with a biological target and/or to produce an effect on a biological target. For example, bioactivity can include, without limitation, elicitation of a stimulatory, inhibitory, regulatory, toxic or lethal response in a biological target. The biological target can be a molecule or a cell. For example, a bioactivity can refer to the ability of an active agent to modulate the effect/activity of an enzyme, block a receptor, stimulate a receptor (e.g., including olfactory receptors and taste receptors), modulate the expression level of one or more genes, modulate cell proliferation, modulate cell division, modulate cell morphology, or any combination thereof. In some instances, a bioactivity can refer to the ability of a compound to produce a toxic effect in a cell.

As used herein, the term “original bioactivity,” in reference to an active agent, generally means the bioactivity of the agent before the agent is introduced into the composition described herein. In some embodiments, the original bioactivity can be as measured immediately before or immediately after the agent is introduced into the composition described herein. That is, the original bioactivity of an active agent can be measured, for example, before the agent is introduced into the composition or within about 20 minutes, before or after the active agent is introduced into the composition. In some instances, the original bioactivity of an active agent can be measured, for example, about 10 seconds, about 15 seconds, about 20 seconds, about 25 seconds, about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes, before or after the active agent is introduced into a silk fibroin matrix.

In some embodiments, the term “original bioactivity” refers to the maximum bioactivity of an active agent, e.g., bioactivity measured immediately after activation of the active agent, e.g., by reconstitution or by increasing the temperature. For example, if the active agent is initially in powder, the original bioactivity of the active agent can be measured immediately after reconstitution. The term “original bioactivity” includes bioactivity of an active agent measured under conditions specified by the manufacturer. Methods for assaying bioactivity of an active agent are known in the art, e.g., by mass spectrometry.

The term “freeze-thaw cycles” is used herein to describe a series of alternating freezing and thawing, and also encompasses a series of alternating frozen (solid) and fluid state. For example, one freeze-thaw cycle involves a change of state between a frozen (solid) state and a fluid state. The time interval between freezing and thawing, or frozen and fluid state, can be any period of time, e.g., hours, days, weeks or months. For example, once an active agent composition has been frozen or is in a frozen state, it can be continually stored in the frozen state at sub-zero temperatures, e.g., between about −20° C. and −80° C., until it needs to be thawed for use again. Freezing of a composition can be performed rapidly, e.g., in liquid nitrogen, or gradually, e.g., in a freezing temperature, e.g., between about −20° C. and −80° C. Thawing of a frozen composition can be performed at any temperature above 0° C. rapidly, e.g., at room temperature, or gradually, e.g., on ice.

The storage-stable compositions described herein can protect the active agent from deactivation and/or degradation due to temperature fluctuation or freeze-thaw cycle(s), and/or eliminate the need for refrigeration. In some embodiments, the storage-stable composition described herein can also stabilize the active agent when it is exposed to light or a relative humidity of at least about 10% or more. Thus, in some embodiments, the active agent (e.g., a volatile, hydrophobic, and/or lipophilic agent) present in the second immiscible phase (e.g., lipid droplets) of the composition or the silk particle can retain at least about 30% of its original bioactivity and/or original loading after the composition is also maintained under exposure to light, e.g., light of different wavelengths and/or from different sources. In some embodiments, the compositions described herein can be maintained under exposure to UV or infra-red irradiation. In some embodiments, the compositions described herein can be maintained under visible lights.

In some embodiments, the active agent (e.g., a volatile, hydrophobic, and/or lipophilic agent) present in the second immiscible phase (e.g., lipid droplets) of the composition or the silk particle can retain at least about 30% of its original bioactivity and/or original loading after the composition is also maintained at a relative humidity of at least about 10% or more, e.g., at least about 20%, at least about 30%, at least about 40%, at least about 50%, at least about 60%, at least about 70%, at least about 80%, at least about 90%, at least about 95% or higher. The term “relative humidity” as used herein is a measurement of the amount of water vapor in a mixture of air and water vapor. It is generally defined as the partial pressure of water vapor in the air-water mixture, given as a percentage of the saturated vapor pressure under those conditions.

In some embodiments, the silk-based material or composition can be in a dried-state. As used herein and throughout the specification, the term “dried state” refers to a state of a composition having water content of no more than 50% or lower, including, e.g., no more than 40%, no more than 30%, no more than 20%, no more than 10%, no more than 5%, no more than 1% or lower. In some embodiments, the silk-based material or composition in a dried-state is substantially free of water. Water can be removed from the silk-based material or composition described herein by any methods known in the art, e.g., air-drying, lyophilization, autoclaving, and any combinations thereof. In some embodiments, the silk-based material or composition can be lyophilized.

### Methods of Producing a Silk Particle or a Composition Described Herein

Methods for producing a silk particle described herein or a composition described herein are also provided. For example, the compositions described herein can be, in general, produced by a process comprising forming an emulsion of the second immiscible phase (e.g., lipid or oil droplets) dispersed in a silk-based material. Silk can act as an emulsifier to stabilize the emulsion of lipid or oil droplets, and thus no addition of emulsifiers is needed.

The lipid droplet(s)-loaded silk particles described herein can be produced by any methods known in the art. For example, in some embodiments, hollow silk particles can be produced, e.g., using the phase separation method as described in International Patent App. No. WO 2011/041395, or the lipid-template guided fabrication method as described in International Patent App. No. WO 2008/118133, followed by immersion in an oil solution for loading/diffusion of oil into the silk particles. In some embodiments, an emulsion of lipid/oil droplets in an aqueous silk solution can be subjected to a freeze-dry process, thereby forming silk-coated lipid/oil particles. In some embodiments, sonication and/or freeze-thawing process can be applied to the emulsion to produce lipid/oil droplets of smaller sizes dispersed in the silk-based material. The silk-coated lipid/oil particles can be used directly or alternatively, suspended in an aqueous medium for further encapsulation within a silk-based matrix, which can in turn produce silk particles loaded with a plurality of silk-coated lipid/oil particles.

While the compositions and/or silk particles described herein can be produced by the methods known in the art, a novel method was developed to produce the silk particles described herein, wherein the method can be controlled to produce a silk particle encapsulating one or more lipid droplets therein. The method comprises (a) providing an emulsion of non-aqueous droplets dispersed in a silk solution undergoing a sol-gel transition (where the silk solution remains in a mixable state); and (b) adding a pre-determined volume of the emulsion into a non-aqueous phase. The silk solution forms in the non-aqueous phase at least one silk particle entrapping at least one of the non-aqueous droplets therein.

In some embodiments, the emulsion in step (a) can be produced by adding a non-aqueous, immiscible phase into the silk solution, thereby forming an emulsion of non-aqueous droplets dispersed in the silk solution. In some embodiments, the silk solution can be treated to induce a sol-gel transition prior to addition of the non-aqueous, immiscible phase into the silk solution. In other embodiments, the non-aqueous, immiscible phase can be added into the silk solution before treating the mixture to induce a sol-gel transition. The non-aqueous, immiscible phase can be any fluid that is not miscible and/or form an interface with the silk solution, e.g., including, but not limited to lipid components, oils, polymers (e.g., polyvinyl alcohol, poly(ethylene glycol), and PLURONICS®), organic solvents, and any combinations thereof.

In some embodiments, the non-aqueous, immiscible phase can comprise lipid components, e.g., but not limited to, oil, fatty acids, glycerolipids, glycerophospholipids, sphingolipids, saccharolipids, polyketides, sterol lipids and prenol lipids. In some embodiments, the non-aqueous, immiscible phase excludes lipid components that can form a liposome under liposome-forming conditions. Examples of such lipid component that can be excluded include, but are not limited to, phosphatidylcholine (PC), phosphatidylethanolamine (PE), phosphatidic acid (PA), phosphatidylglycerol (PG), sterol such as cholesterol, and normatural lipid(s), cationic lipid(s) such as DOTMA (N-(1-(2,3-dioxyloxyl)propyl)-N,N,N-trimethyl ammonium chloride), as well as 1,2-dioleoyl-sn-glycero-3-phosphocholine (DOPC); 1,2-dioleoyl-sn-glycero-3-phophoethanolamine (DOPE); 1,2-dilauroyl-sn-glycero-3-phosphocholine (DLPC); and 1,2-dimyristoyl-sn-glycero-3-phosphocholine (DMPC); and any combinations thereof. In some embodiments, the lipid component can exclude phospholipids. In some embodiments, the lipid component can exclude glycerophospholipids.

In some embodiments, the non-aqueous, immiscible phase is oil as defined earlier. In one embodiment, the non-aqueous, immiscible phase is plant-derived oil, e.g., sunflower oil.

The volume of the non-aqueous, immiscible phase added to the silk solution can vary, e.g., depending on particle size, and/or concentration of non-aqueous droplets dispersed in the silk solution. In some embodiments, the non-aqueous, immiscible phase can be added to the silk solution at a non-aqueous, immiscible phase: silk volumetric ratio of about 1:1 to about 1:500, or about 1:2 to about 1:250, or about 1:3 to about 1:100, or about 1:5 to about 1:50.

In some embodiments, the non-aqueous droplets in the emulsion can further comprise at least one or more (e.g., 1, 2, 3, 4, or more) active agents, e.g., volatile, hydrophobic, and/or lipophilic agents defined herein. In some embodiments, the active agent(s), e.g., volatile, hydrophobic, and/or lipophilic agent(s), can be added into the non-aqueous, immiscible phase before adding the non-aqueous immiscible phase into the silk solution to form an emulsion. Examples of a volatile, hydrophobic and/or lipophilic agent can include, but are not limited to, a therapeutic agent, a nutraceutical agent, a cosmetic agent, a food additive (e.g., a coloring agent, a flavoring substance), an odor-releasing substance (e.g., fragrance), a probiotic agent, a dye, an aromatic compound, an aliphatic compound (e.g., alkane, alkene, alkyne, cyclo-alkane, cyclo-alkene, and cyclo-alkyne), or any combinations thereof.

In some embodiments, the active agent(s) to be included in the non-aqueous droplets can be provided in a form of an oil, e.g., volatile oil or essential oil, which is generally a concentrated hydrophobic oil containing volatile aroma compounds from plants.

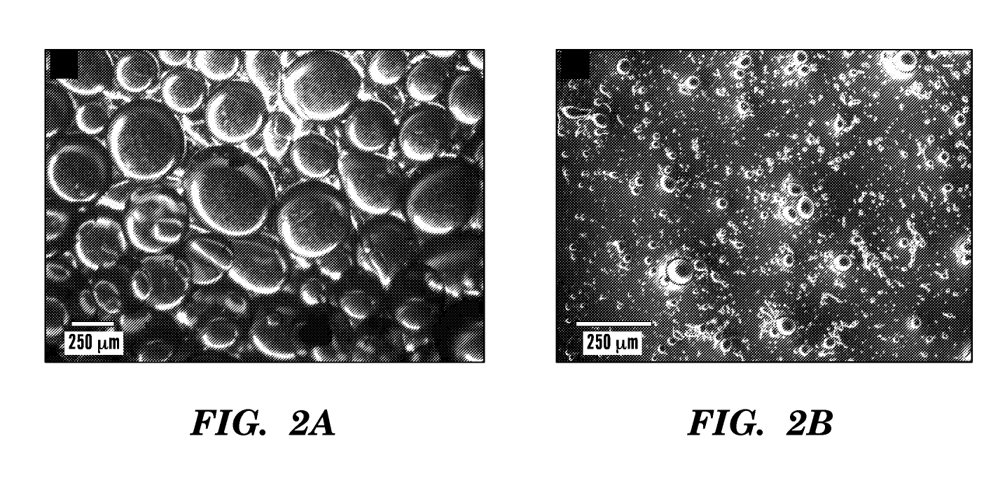

In some embodiments, the silk solution comprising non-aqueous droplets (an emulsion of non-aqueous droplets dispersed in the silk solution) can be subjected to sonication and/or freeze-thawing process. Without wishing to be bound by theory, the sonication and/or freeze-thawing process can decrease the size of the non-aqueous droplets dispersed in the silk solution. By way of example only, prior to sonication, an emulsion of oil mixed with an aqueous silk solution can exhibit an average oil droplet diameter of about 100 μm to about 700 μm (e.g., ˜420 μm as shown in FIG. 2A). Gentle sonication (e.g., ˜10% amplitude for about 5 seconds) of the emulsion reduced the average oil droplet diameter to less than 50 μm, or less than 25 μm, or less than 10 μm, or less than 5 μm or lower (e.g., less than 25 μm as shown in FIG. 2B).

As used herein, the term “sol-gel transition” refers to a state of a silk solution, which is presented as a flowable liquid for a certain period of time and is then changed into a gel after the certain period of time. In accordance with embodiments described herein, a silk solution with a sol-gel transition can remain in the solution phase long enough to perform the double emulsion and is then changed into a gel, thereby encapsulating the oil droplets therein. Accordingly, the sol-gel transition of the silk solution comprising the oil droplets can last for a period of time that is sufficient to remain as an emulsion or in solution state when it is aliquoted into a non-aqueous phase (e.g., but not limited to, oil, and organic solvent such as polyvinyl alcohol) and then form a gel particle entrapping the oil droplets in the non-aqueous phase (e.g., but not limited to, oil, and organic solvent such as polyvinyl alcohol). In some embodiments, the sol-gel transition can last for at least about 5 seconds, at least about 10 seconds, at least about 20 seconds, at least about 30 seconds, at least about 40 seconds, at least about 50 seconds, at least about 60 seconds or more. In some embodiments, the sol-gel transition can last for at least about 5 minutes, at least about 10 minutes, at least about 15 mins, at least about 30 mins, at least about 1 hour, or at least about 2 hours or more. In some embodiments, the sol-gel transition can last for at least about 6 hours, at least about 12 hours, at least about 1 day, at least about 2 days or more. In some embodiments, the sol-gel transition can last for no more than 2 days, no more than 1 day, no more than 12 hours, no more than 6 hours, no more than 3 hours, no more than 2 hours, no more than 1 hour, no more than 30 minutes, no more than 15 minutes, no more than 10 minutes, no more than 5 minutes, no more than 1 minute, or less.

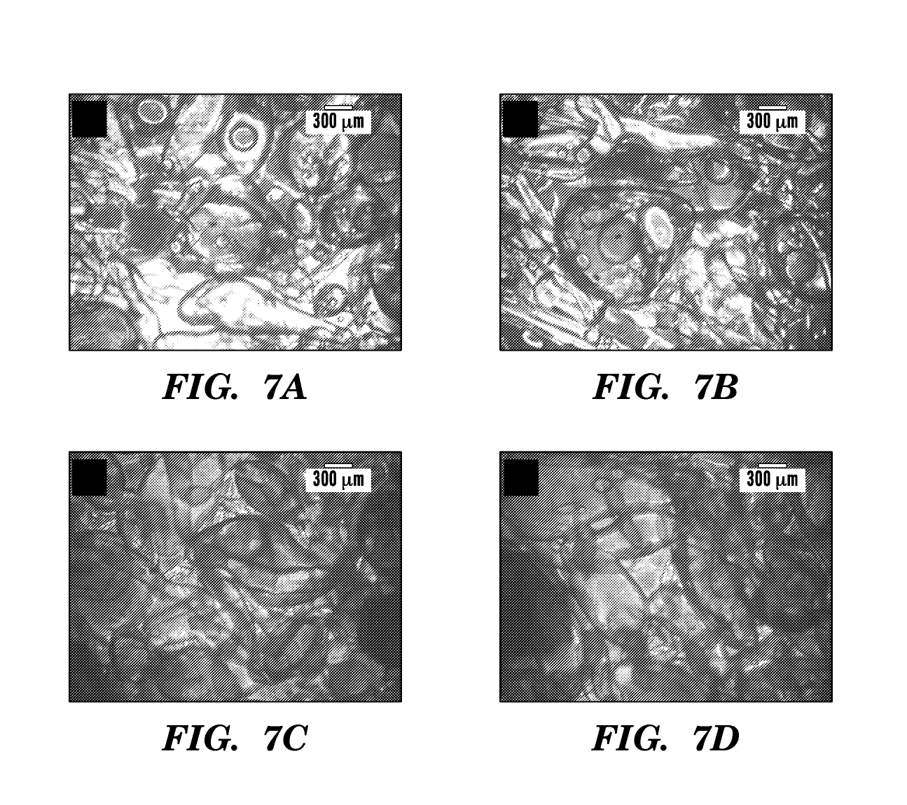

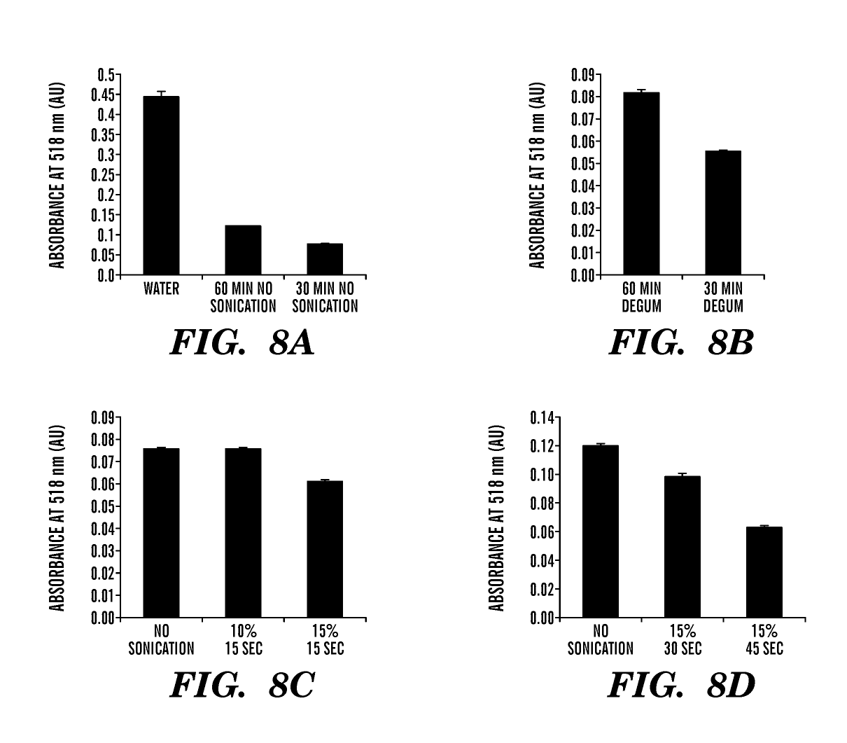

The sol-gel transition of the silk solution can be induced by any method that is known to induce a conformation change in silk fibroin, including, e.g., by electrogelation, reduced pH, shear stress, vortexing, sonication, electrospinning, salt addition, air-drying, water annealing, water vapor annealing, alcohol immersion, and/or any other silk gelation methods. In some embodiments, the sol-gel transition of the silk solution can be induced by sonication. One skilled in the art can control sonication process to tune for various duration of sol-gel transition, see, e.g., U.S. Pat. No. 8,187,616, the content of which is incorporated herein by reference in its entirety. In one embodiment, the sonication can be performed at an amplitude of about 1% to about 50%, or about 5% to about 25%, or about 10% to about 15%. In some embodiments, the sonication duration can last for from about 5 sec to about 90 sec, or from about 15 sec to about 60 sec, or from about 30 sec to about 45 sec. The sonication treatment parameters (e.g., amplitude, time, or both) can be controlled accordingly to adjust for the desirable material properties of the resulting silk particles (e.g., silk particle size and/or shape, lipid droplet size and/or shape, and/or permeability of the silk as an encapsulant material. By way of example only, as shown in Example 1, as the sonication intensity increases (e.g., by increasing amplitude and/or time duration such as ˜10% amplitude for ˜15 seconds in FIGS. 7A-7B, compared to ˜15% for ˜15 seconds in FIGS. 7C-7D), the resulting silk particles appeared to be more elongated and irregular. In addition, the permeability of the silk-based material to an active agent present in the interior oil phase decreased (FIGS. 8C-8D).

In addition to the sonication treatment parameters, other control parameters for the material properties of the silk particles include, e.g., but not limited to, silk solution properties (e.g., composition, concentration, solution viscosity, silk degumming time), particle fabrication parameters (e.g., presence or absence of particle coating(s), volumetric ratio of silk fibroin and oil phase, aliquot volume of a silk-based emulsion (dispersion of oil droplets in the sol-gel silk solution) added to a continuous phase (e.g., oil or organic solvent such as polyvinyl alcohol)), hydrophobicity of an active agent to be encapsulated, post-treatment of the silk particle (e.g., but not limited to beta-sheet inducing treatment such as lyophilization, water annealing, and water vapor annealing), if any, and any combinations thereof.

By way of example only, the concentration of the silk solution can, in part, influence the lipid encapsulation configuration. For example, higher concentrations of the silk solution can produce a dispersion of multiple oil droplets suspended throughout the silk-comprising phase (termed as “a microsphere”), while lower concentrations of the silk solution can result in a “microcapsule” configuration, where one large lipid droplet surrounded by a silk capsule is incorporated in each individual particle. Accordingly, the silk solution used for producing a silk-based material can have any concentration, e.g., ranging from about 0.5% (w/v) to about 30% (w/v). In some embodiments, it can be desirable to use a silk concentration lower than 0.5% (w/v) or higher than 30% (w/v) for intended mechanical properties. In some embodiments, the silk solution can have a concentration of about 1% (w/v) to about 15% (w/v), or about 2% (w/v) to about 7% (w/v).

In some embodiments, the concentration of the silk solution selected can depend on the degumming time of silk cocoons. In some embodiments, the degumming time of silk cocoons can range from about less than 5 minutes to about 60 minutes. Without wishing to be bound by theory, the viscosity of the silk solution generally increases with decreasing degumming time. Thus, in some embodiments, in order to maintain a certain solution viscosity, higher concentration of a silk solution produced from silk with longer degumming time can be desired. In some embodiments where silk cocoons has been degummed for a short period of time, e.g., less than 15 minutes, the concentration of the silk solution can be as low as 0.5% to maintain structural integrity of the silk-based material. See, e.g., International Appl. No. PCT/US13/49740 filed Jul. 9, 2013 for information about using gently-degummed silk in formation of different silk-based materials.

In some embodiments, the silk solution can further comprise at least one or more active agent as described herein. For example, in some embodiments, the silk solution can further comprise at least two, at least three, at least four, at least five or more active agents as described herein. Thus, in some embodiments, the method can further comprise adding at least one active agent into the silk fibroin solution prior to or after treating the silk solution to induce a sol-gel transition.

In some embodiments, the silk solution can further comprise at least one additive as described herein. In some embodiments, the silk solution can further comprise at least one of biocompatible polymers or biopolymers; plasticizers (e.g., glycerol); emulsion stabilizers (e.g., lecithin, and/or polyvinyl alcohol), surfactants (e.g., polysorbate-20); interfacial tension-modulating agents such as surfactants (e.g., salt); beta-sheet inducing agents (e.g., salt); and detectable agents (e.g., a fluorescent molecule). In one embodiment, the silk solution can further comprise an emulsion stabilizer (e.g., lecithin, and/or polyvinyl alcohol).

By adding a pre-determined volume of the emulsion from step (a) into the non-aqueous phase (e.g., an oil phase or an organic solvent such as polyvinyl alcohol), e.g., dropwise via an extrusion-like process, the size of the resulting silk particle can be controlled. For example, the pre-determined volume of the emulsion can substantially correspond or proportional to a desirable size of the silk particle. An extrusion-like process can be characterized by precise control of particle size and composition loading. For example, an extrusion-like process can include pipetting or injecting controlled volumes of a known composition into a continuous phase, e.g., an oil phase. In some embodiments, microfluidics can be used to produce smaller silk particles, as has been described for other biomaterial microparticles (Chu et al., 2007; Tan and Takeuchi, 2007; Ren et al., 2010).

While the emulsion (of non-aqueous droplets dispersed in the silk solution) is generally added into a non-aqueous phase (e.g., an oil phase or an organic solvent such as polyvinyl alcohol) to form a silk particle encapsulating at least one non-aqueous droplet, in some embodiments, the emulsion can be added to an aqueous solution comprising a surfactant (any molecule that can reduce interfacial tension, e.g., but not limited to polysorbate-20). In one embodiment, the emulsion can be added to a salt solution (e.g., but not limited to sodium chloride (NaCl)) comprising a surfactant (e.g., but not limited to polysorbate-20). In this embodiment, not only can a silk particle form in the salt solution, beta-sheet can also form in silk fibroin in the presence of the salt (e.g., NaCl is known to induce beta sheet in silk fibroin).

In some embodiments, the methods can further comprise isolating the formed silk particle from the non-aqueous phase. Methods for isolating the dispersed particles from a continuous phase of an emulsion are known in the art, e.g., filtration and/or centrifugation, and can be used herein.

In some embodiments, the method can further comprise selecting the formed silk particle of a specific size, or within a selected size distribution.

In some embodiments, the silk particles can be maintained in a rubbery, hydrated gelled state. In some embodiments, the method can further comprise subjecting the silk particle to a post-treatment. The post-treatment can include any process that changes at least one material property of the silk particle (e.g., but not limited to, solubility, porosity, and/or mechanical property of the resulting silk particles). For example, in some embodiments, the post-treatment can include a dehydration process (e.g., by drying or lyophilization) to produce a silk particle in a dried state. In some embodiments, lyophilization of the silk particle can introduce porous structure in silk matrix therein. In other embodiments, the post-treatment can include a process that further induces a conformational change in silk fibroin in the particle. The conformational change in silk fibroin can be induced, for example, but not limited to, one or more of lyophilization or freeze-drying, water annealing, water vapor annealing, alcohol immersion, sonication, shear stress, electrogelation, pH reduction, salt addition, air-drying, electrospinning, stretching, or any combination thereof. In some embodiments, the silk particle and/or the silk-based composition can be subjected to freeze-drying. In some embodiments, the silk particle and/or the silk-based composition can be subject to an annealing process as described in detail below, e.g., water vapor annealing.

In some embodiments, the method can further comprise forming on an outer surface of the silk particle a coating. The coating can be used to act as a barrier to maintain moisture, and/or increase the retention of a volatile, hydrophobic, and/or lipophilic agent encapsulated in interior oil phases surrounded by the silk-based material. Alternatively or additionally, the coating can be used to control the release of the volatile, hydrophobic, and/or lipophilic agent encapsulated in interior oil phases surrounded by the silk-based material. In some embodiments, the coating can be used to control the optical property of the composition described herein, e.g., a coating to reduce reflection. In some embodiments, the coating can be used to improve the smoothness of the particle surface. In some embodiments, the coating can be used to improve targeting of the silk particle to a specific cell.

The coating can be applied to the outer surface of the silk particle by any methods known in the art, e.g., dip-coating, spraying, chemical vapor deposition, physical vapor deposition, plating, electrochemical method, sol-gel, optical coating, powder coating, powder slurry coating, centrifugation, and any combinations thereof.

Any biocompatible polymer described herein can be used for coating the outer surface of the silk particles described herein. In some embodiments, the coating can comprise a hydrophilic polymer. Examples of hydrophilic polymer include, but are not limited to, homopolymers such as cellulose-base polymer, protein-based polymer, water-soluble vinyl-base polymer, water-soluble acrylic acid-base polymer and acrylamide-base polymer, and synthetic polymers such as crosslinked hydrophilic polymer, e.g., poly(ethylene oxide).

In some embodiments, the coating can comprise a silk fibroin layer. See, e.g., International App. No. WO 2007/016524 for description of an example method to form silk coating. For example, a silk coating can be formed by contacting the outer surface of the silk particle with a silk solution and inducing a conformational change in silk fibroin, e.g., using any of the art-recognized methods and/or any methods described herein.

In some embodiments, the coating can comprise a hydrophilic polymer layer overlaid with a silk layer. In these embodiments, the hydrophilic polymer layer can comprise poly(ethylene oxide) (PEO). To form a coating comprising a hydrophilic polymer layer overlaid with a silk layer, by way of example only, the outer surface of the silk particle can be contacted with a hydrophilic solution to form a hydrophilic polymer layer, and the resulting hydrophilic polymer layer can then be contacted with a silk solution to form a silk coating over the hydrophilic polymer coating.

Without wishing to be bound by theory, while the PEO is highly viscous and can function as a water retention barrier, the addition of silk coating can provide protection of the encapsulated substance. The silk layer can serve to limit diffusion of PEO and prevent rapid water loss. Without wishing to be bound by theory, the combined PEO/silk coating can help maintain hydration around the silk particles and prevent premature release of volatile agents such as fragrance.

In some embodiments, the coating can further comprise an additive as described herein. For example, the coating can further comprise a contrast agent and/or a dye.

Exemplary methods of using the silk particles and/or silk-based compositions described herein

Different embodiments of the compositions described herein can be used, for example, in tissue engineering such as to model a tissue with high lipid content, or in controlled release and/or stabilization of a volatile, hydrophobic and/or lipophilic agent as described herein. Accordingly, methods of using one or more embodiments of the compositions are also provided herein. For example, some embodiments of the compositions described herein can be used to stabilize an active agent present in the second immiscible phase of the composition (e.g., a volatile, hydrophobic and/or lipophilic agent present in an interior oil phase). The silk particles and/or silk-based compositions can be used as a format to store and stabilize or maintain the bioactivity and/or loading of labile and/or volatile materials at room temperature or above. Thus, in some embodiments, the silk particles and/or silk-based compositions can be used to maintain the stability of an active agent under a specific condition and/or used as a depot for an active agent administered to a subject. Accordingly, in one aspect, the method of use can comprise maintaining at least one composition (including a storage-stable composition described herein) or at least one silk particle described herein, wherein the active agent present in the second immiscible phase of the composition or the silk particle can retain at least a portion of its original bioactivity and/or original loading (e.g., at least about 30% or higher, including, e.g., at least about 40%, at least about 50%, at least about 60%, at least about 70%, at least about 80%, or higher) when the composition is (a) subjected to at least one freeze-thaw cycle, or (b) maintained for at least about 24 hours at a temperature of about room temperature or above, or (c) both (a) and (b).

In some embodiments, the composition can be maintained for at least about 1 month or longer, e.g., at least about 2 months or longer, at least about 3 months, at least about 4 months, at least about 5 months, or longer.

Additionally or alternatively, some embodiments of the compositions described herein can be used to controllably release an active agent from the second immiscible phase of the composition (e.g., a volatile, hydrophobic and/or lipophilic agent present in an interior oil phase). Thus, in one aspect, the method of use can comprise maintaining at least one composition (including a storage-stable composition described herein) or at least one silk particle described herein, wherein the silk-based material is permeable to said at least one active agent such that the active agent can be released through the silk-based material into an ambient surrounding at a pre-determined rate. In some embodiments, the pre-determined rate of the release can be controlled by, for example, adjusting an amount of beta-sheet conformation of silk fibroin present in the silk-based material, porosity of the silk-based material, or a combination thereof. Methods for producing porous silk materials are known in the art, e.g., by porogen-leaching method, and/or freeze-drying.

The composition can be maintained at any environmental condition. For example, in some embodiments, the composition can be maintained at about room temperature. In other embodiments, the composition can be maintained at a temperature of about 37° C. or greater. In some embodiments, the composition can be maintained under exposure to light. In some embodiments, the composition can be maintained at a relative humidity of at least about 10% or higher, including, e.g., at least about 20%, at least about 30%, at least about 40%, at least about 50%, at least about 60%, at least about 70%, at least about 80%, at least about 90%, or above.

The silk particles and/or silk-based compositions described herein can also be used to deliver an active agent, e.g., a labile and/or volatile material. The method of delivering an active agent (e.g., a volatile, hydrophobic and/or lipophilic agent) comprises applying or administering to a subject at least one composition (including a storage-stable composition described herein) or at least one silk particle described herein, said silk-based material of the composition or silk particle being permeable to the active agent such that the active agent can be released through the silk-based material, at a pre-determined rate, upon application or administration of the composition to the subject.

In some embodiments, the active agent can be released to an ambient surrounding. The term “ambient surrounding” described herein refers to a surrounding of a silk particle or silk-based composition described herein, depending on where the silk particle or silk-based composition is placed or applied. Depending on purposes of the applications and/or application sites, in some embodiments, the active agent present in the second immiscible phase of the composition (e.g., a volatile, hydrophobic and/or lipophilic agent present in an interior oil phase) can be released to an ambient surrounding, e.g., ambient air. In these embodiments, the composition can be applied to the subject topically. In one embodiment, the composition can be applied on a skin or surface of a subject. The subject can be a living subject, e.g., a mammalian subject, or it can be a physical object, such as an article of manufacture.

Alternatively, the active agent present in the second immiscible phase of the composition (e.g., a volatile, hydrophobic and/or lipophilic agent present in an interior oil phase) can be released to a target biological cell of a subject when the composition is applied or administered in vivo. In these embodiments, the composition can be applied or administered to the subject orally or parenterally.

### Inducing a Conformational Change (e.g., Beta-Sheet Formation) in Silk Fibroin

In some embodiments, the silk particles and/or silk-based compositions described herein can be made water-insoluble, e.g., by increasing the beta-sheet content in silk fibroin. There are a number of different methods for inducing a conformational change (e.g., beta sheet formation) in silk fibroin in a silk-based material. Without wishing to be bound by a theory, inducing a conformational change in silk fibroin can alter the crystallinity of the silk fibroin in the silk-based material, e.g., Silk II beta-sheet crystallinity. This can alter the rate of release of a molecule, if any, encapsulated in the silk matrix and/or alter the rate of degradation of the silk matrix (and in turn the release of the incorporated lipid phases). A conformational change in silk fibroin can be induced by any method known in the art, including, but not limited to, alcohol immersion (e.g., ethanol, methanol), water annealing, water vapor annealing heat annealing, shear stress, ultrasound (e.g., by sonication), pH reduction (e.g., pH titration and/or exposing a silk matrix to an electric field), freeze drying, and any combinations thereof. For example, beta-sheet conformation in silk fibroin can be done by one or more methods, including but not limited to, controlled slow drying (Lu et al., 10 Biomacromolecules 1032 (2009)); water annealing (Jin et al., 15 Adv. Funct. Mats. 1241 (2005); Hu et al., 12 Biomacromolecules 1686 (2011)); stretching (Demura & Asakura, 33 Biotech & Bioengin. 598 (1989)); compressing; solvent immersion, including methanol (Hofmann et al., 111 J Control Release. 219 (2006)), ethanol (Miyairi et al., 56 J. Fermen. Tech. 303 (1978)), glutaraldehyde (Acharya et al., 3 Biotechnol J. 226 (2008)), and 1-ethyl-3-(3-dimethyl aminopropyl) carbodiimide (EDC) (Bayraktar et al., 60 Eur J Pharm Biopharm. 373 (2005)); pH adjustment, e.g., pH titration and/or exposing a silk matrix to an electric field (see, e.g., U.S. Patent App. No. US2011/0171239); heat treatment; shear stress (see, e.g., International App. No.: WO 2011/005381), ultrasound, e.g., sonication (see, e.g., U.S. Patent Application Publication No. U.S. 2010/0178304 and International App. No. WO2008/150861); and any combinations thereof. Content of all of the references listed above is incorporated herein by reference in their entirety.

In some embodiments, the silk particles and/or silk-based compositions described herein can comprise a labile and/or volatile active agent that may require milder silk processing methods. Accordingly, in some embodiments, beta sheet formation in the silk particles and/or silk-based compositions can be induced by water annealing. There are a number of different methods for water annealing. One method of water annealing involves treating solidified but soluble forms of silk fibroin with water vapor. Without wishing to be bound by a theory, it is believed that water molecules act as a plasticizer, which allows chain mobility of fibroin molecules to promote the formation of hydrogen bonds, leading to increased beta sheet secondary structure. This process is also referred to as “water vapor annealing” herein. Without wishing to be bound by a theory, it is believed that physical temperature-controlled water vapor annealing (TCWVA) provides a simple and effective method to obtain refined control of the molecular structure of silk biomaterials, e.g., silk matrix disclosed herein. The silk matrix can be prepared with control of beta-sheet crystallinity, from low content using conditions at 4° C. (α helix dominated silk I structure), to high content of ˜60% crystallinity at 100° C. (β-sheet dominated silk II structure). This physical approach covers the range of structures previously reported to govern crystallization during the fabrication of silk materials, yet offers a simpler, green chemistry, approach with tight control of reproducibility. Temperature controlled water vapor annealing is described, for example, in Hu et al., Regulation of Silk Material Structure By Temperature Controlled Water Vapor Annealing, Biomacromolecules, 2011, 12(5): 1686-1696, content of which is incorporated herein by reference in its entirety.

Another way of annealing is by slow, controlled evaporation of water from silk fibroin in the silk material/matrix. Slow, controlled, drying is described in, for example, Lu et al., Acta. Biomater. 2010, 6(4): 1380-1387.

Without wishing to be bound by a theory, it is believed that water annealing provides a simple and effective method to obtain refined control of the molecular structure of silk fibroin in silk-based materials and compositions. Using water annealing, the silk-based material can be prepared with control of beta-sheet crystallinity, from a low content using conditions at 4° C. (a helix dominated silk I structure), to a high content of ˜60% crystallinity (β-sheet dominated silk II structure) using condition at 100° C. This physical approach covers the range of structures previously reported to govern crystallization during the fabrication of silk materials, yet offers a simpler, green chemistry, approach with tight control of reproducibility. Water or water vapor annealing is described, for example, in PCT/US2004/011199, filed Apr. 12, 2004; PCT/US2005/020844, filed Jun. 13, 2005; Jin et al., Adv. Funct. Mats. 2005, 15: 1241; and Hu et al., 2011, 12(5): 1686-1696, content of all of which is incorporated herein by reference in their entirety. Accordingly, in some embodiments, the silk-based material comprises beta-sheet crystallinity of at least 10%, e.g., 15%, 20%, 25%, 30%, 35%, 40%, 45%, 50%, 55%, 60%, 65%, 70%, 75%, 70%, 85%, 90%, 95% or more, but not 100% (i.e., not all the silk fibroin is in a beta-sheet conformation). In some embodiments, all of the silk fibroin in the composition is in a beta-sheet conformation, i.e., 100% beta-sheet crystallinity. The terms beta-sheet crystallinity and silk II are used interchangeably herein. Thus, a stated beta-sheet crystallinity % also means the amount of silk fibroin that is in the silk II conformation.

The annealing step can be performed within a water vapor environment, such as in a chamber filled with water vapor, for different periods of time. Without wishing to be bound by a theory, length of annealing effects the amount of beta-sheet crystallinity obtained in the silk-based material. Accordingly, typical annealing time periods can range from seconds to days. In some embodiments, the annealing is for a period of seconds to hours. For example, annealing time can range from a few seconds (e.g., about 5, 10, 15, 20, 25, 30, 35, 40, 45, 50, 55, or 60 seconds) to about 2, 6, 12, 24, 36, or 48 hours.

The temperature of the water vapor used in the annealing process effects the amount of bets-sheet crystallinity obtained. See HU et al., Biomacromolecules, 12: 1686-1696. Accordingly, the annealing can be performed at any desired temperature. For example, the annealing can be performed with a water vapor temperature from about 4° C. to about 120° C. Optimal water vapor to obtain a required amount of beta-sheet crystallinity in the silk matrix can be calculated based on equation (I):

C=a(1−exp(−k.T)) (I)

wherein C is beta-sheet crystallinity, a is 62.59, k is 0.028 and T is annealing temperature. See HU et al., Biomacromolecules, 12: 1686-1696.

Without wishing to be bound by a theory, the pressure under which the annealing takes place can also influence the degree or amount of beta-sheet crystallinity. In some embodiments, the contacting can be performed in a vacuum environment.

Relative humidity under which the annealing takes place can also influence the degree or amount of beta-sheet crystallinity. Relative humidity under which the silk-based material is contacted with water or water vapor can range from about 5% to 100%. For example, relative humidity can be from about 5% to about 95%, from about 10% to about 90%, or from about 15% to about 85%. In some embodiments, relative humidity is 90% or higher.

Another useful method for inducing beta-sheet formation in the silk fibroin is to subject the silk-based material to dehydration by the use of organic solvent, such as alcohols, e.g., methanol, ethanol, isopropyl, acetone, etc. Such solvent has an effect of dehydrating silk fibroin, which promotes “packing” of silk fibroin molecules to form beta sheet structures. In some embodiments, a silk-based material can be treated with an alcohol, e.g., methanol, ethanol, etc. The alcohol concentration can be at least 10%, at least 20%, at least 30%, at least 40%, at least 50%, at least 60%, at least 70%, at least 80%, at least 90% or 100%. In some embodiment, alcohol concentration is about 90%.

Regardless of the methods employed to induce beta-sheet formation, the treated silk fibroin can have high degree of crystallinity such that it becomes insoluble. In some embodiments, “high degrees of crystallinity” refers to beta sheet contents of between about 20% and about 70%, e.g., about 20%, about 25%, about 30%, about 35%, about 40%, about 45%, about 50%, about 55%, about 60%, about 65% and about 75%.

In some embodiments, inducing beta-sheet formation can provide silk-based material can comprising a silk II beta-sheet crystallinity content of at least about 20%, at least about 30%, at least about 40%, at least about 50%, at least about 60%, at least about 70%, at least about 80%, at least about 90%, or at least about 95% but not 100% (i.e., all the silk is present in a silk II beta-sheet conformation). In some embodiments, the silk-based material can have a Silk II beta-sheet crystallinity of 100%.

Using the methods and compositions disclosed in the present disclosure, one can obtain a desired beta-sheet crystallinity in the silk-based material while the active agent maintains at least 50% (e.g., 50%, 55%, 60%, 65%, 70%, 75%, 80%, 85%, 90%, 95% or more) of its original activity. Without limitations, the active agent can be distributed in the silk-based material, encapsulated by the matrix, coated by the matrix, or any combinations thereof.

Examples of Active Agents for Encapsulation in Silk-Based Material and/or in Silk-Immiscible Compartments or Droplets, e.g., Oil

As used herein, the term “active agent” refers to any molecule, compound or composition, an activity of which is desired to be maintained when such molecule, compound, or composition is incorporated in a silk-based material and/or in a silk-immiscible compartment (e.g., lipid-comprising compartments, e.g., oil). Without limitations, the active agent can be selected from the group consisting of small organic or inorganic molecules; saccharides; oligosaccharides; polysaccharides; peptides; peptide analogues and derivatives; peptidomimetics; proteins; antigens; antibodies; antigen binding fragments of antibodies; enzymes; immunogens; vaccines; nucleic acids, e.g., DNA, RNA, oligonucleotides, polynucleotides, siRNA, shRNA, modRNA (including LNA) antisense oligonucleotides, aptamers, ribozymes, activating RNA, decoy oligonucleotides, and the like); nucleic acid analogs and derivatives, e.g., peptide nucleic acids, locked nucleic acids, modified nucleic acids, and the like); antibiotics; therapeutic agents; cells; viruses; bacteria; extracts made from biological materials such as bacteria, viruses, plants, fungi, or animal cells; animal tissues; naturally occurring or synthetic compositions; and any combinations thereof. In some embodiments, the active agent encapsulated in the first immiscible phase can be more soluble in the first immiscible phase than in the second immiscible phase. In some embodiments, the active agent encapsulated in the second immiscible phase can be more soluble in the second immiscible phase than in the first immiscible phase.

Methods are well known in the art for obtaining compositions comprising silk fibroin and active agent. However, methods of preparing compositions comprising silk fibroin and active agents and retaining at least some of the original bioactivity and/or original loading of the active agent are not straight forward. Especially, when the composition under needs post-processing after preparation. Many active agents are sensitive to the conditions used in fabricating the silk-based matrices and can lose their activity once becoming encapsulated in the silk matrix. For example, most biological molecules are sensitive to organic solvents, such as alcohol. On coming in contact with an organic solvent, these molecules can lose at least a part of their activity. Thus, conditions relying on organic solvents for producing the composition can be detrimental to active agents to be retained in silk-based matrices

Thus, in some embodiments, the active agent is a biological molecule. As used herein, the term “biological molecule” refers to any molecule known to be found in biological systems and includes, amino acids, proteins, peptides, antibodies, antigen binding fragment of antibodies, nucleic acids (including DNA and RNA), saccharides, polysaccharides and the like. As used herein, biological molecules include those which are naturally occurring as well as those which have been modified using known techniques.

In some embodiments, the active agent is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), or mixtures or combinations thereof, including, for example, DNAnanoplexes.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain combinations of two or more therapeutic agents.

Exemplary therapeutic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

Examples of other active agents include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January; 60(1):119-32; Hersel U. et al. 2003 Biomaterials. November; 24(24):4385-415); biologically active ligands; and substances that enhance or exclude particular varieties of cellular or tissue ingrowth. Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β1, and the like.

While any active agent described herein can be encapsulated in a silk-immiscible phase (being referred to as “second immiscible phase” herein while the “first immiscible phase” described herein comprises a silk-based matrix), in some embodiments, the active agent present in the second immiscible phase can comprise a hydrophobic or lipophilic molecule. As used herein, the term “hydrophobic molecule” refers to a molecule that cannot be completely soluble in water. As used herein, the term “lipophilic molecule” refers to a molecule tending to combine with or dissolve in lipids or fats. Examples of the hydrophobic or lipophilic molecule can include, but are not limited to, a therapeutic agent, a nutraceutical agent (e.g., fat-soluble vitamins), a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

Further, the ratio of silk fibroin to active agent, or the ratio of silk-immiscible phase to active agent can be any desired ratio. For example, the ratio of silk fibroin to active agent, or the ratio of silk-immiscible phase to active agent can range from about 1:1000 to about 1000:1, about 1:500 to about 500:1, about 1:250 to about 250:1, about 1:125 to about 125:1, about 1:100 to about 100:1, about 1:50 to about 50:1, about 1:25 to about 25:1, about 1:10 to about 10:1, about 1:5 to about 5:1, about 1:3 to about 3:1, or about 1:1. The ratio of the silk fibroin to the active agent, or the ratio of silk-immiscible phase to active agent, can vary with a number of factors, including the selection of the active agent, the concentration of the silk fibroin, form of the silk-based material, size of the silk-immiscible phase, and the like. One of skill in the art can determine appropriate ratio of the silk fibroin to the active agent, e.g., by measuring the bioactivity of the active agent at various ratios as described herein.

### Various Forms of Silk-Based Material

As described herein, a silk-based material encapsulating an immiscible phase (optionally comprising an active agent) can be in any form, shape or size. For example, the silk-based material can be a solution, a fiber, a film, a sheet, a mat, a non-woven mat, a mesh, a sponge, a foam, a gel, a hydrogel, a tube, a particle (e.g., a nano- or micro-particle, a gel-like particle), a powder, a scaffold, a 3D construct, a tissue engineered construct, a coating layer on a substrate, or any combinations thereof.

In some embodiments, the silk-based material can be in the form of an injectable composition. By the term “injectable composition”, as used herein, is meant a composition having a suitable viscosity to be readily injected through a conventional cannula, which has an 18 Gauge needle dimension or finer dimensions. In a more specific embodiment, a composition according to the invention is able to pass through a 21 Gauge needle. To comply with these criteria of injectability, the composition according to the present invention should have a viscosity less than about 60,000 cSt.

In some embodiments, the active agent is distributed, homogenously or in homogenously in the silk-based material. In some embodiments, the active agent is encapsulated by the silk fibroin in the silk-based material. In some embodiments, the active agent is coated by a layer of the silk fibroin.

In some embodiments, the silk-based material is in the form of a matrix comprising a lumen or cavity therein and at least a part amount of the active agent is present in the lumen or cavity. In some embodiments, the silk fibroin is in the form of a matrix comprising a lumen or cavity therein and at least a part amount of the active agent is present in the lumen or cavity and at least a part amount of the active agent is distributed in the silk fibroin network itself. In some embodiments, when the matrix comprises a lumen or cavity, at least 5%, (e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98%) of the active agent is present in the lumen or cavity formed by the silk-based material. In some embodiments, the entire amount of the active agent is present in the lumen/cavity.

As indicated above, the silk-based material comprising the active agent can be in any form, shape or size. Accordingly, in some embodiments, the silk-based material is in the form of a fiber. As used herein, the term “fiber” means a relatively flexible, unit of matter having a high ratio of length to width across its cross-sectional perpendicular to its length. Methods for preparing silk fibroin fibers are well known in the art. A fiber can be prepared by electrospinning a silk solution, drawing a silk solution, and the like. Electrospun silk materials, such as fibers, and methods for preparing the same are described, for example in WO2011/008842, content of which is incorporated herein by reference in its entirety. Without limitations, the active agent can be distributed in the silk fibroin matrix of the fiber, present on a surface of the fiber, or any combination thereof.

In some embodiments, the silk-based material comprising the active agent can be in the form of a film, e.g., a silk film. As used herein, the term “film” refers to a flat or tubular flexible structure. It is to be noted that the term “film” is used in a generic sense to include a web, film, sheet, laminate, or the like. In some embodiments, the film is a patterned film, e.g., nanopatterned film. Exemplary methods for preparing silk fibroin films are described in, for example, WO 2004/000915 and WO 2005/012606, content of both of which is incorporated herein by reference in its entirety. Without limitations, the active agent can be distributed in the film, present on a surface of the film, coated by the film, or any combination thereof.

In some embodiments, the silk matrix can be in the form of a silk particle, e.g., a silk nanosphere or a silk microsphere. As used herein, the term “particle” includes spheres; rods; shells; and prisms; and these particles can be part of a network or an aggregate. Without limitations, the particle can have any size from nm to millimeters. As used herein, the term “microparticle” refers to a particle having a particle size of about 1 μm to about 1000 μm. As used herein, the term “nanoparticle” refers to particle having a particle size of about 0.1 nm to about 1000 nm.

It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

Without limitation, there are at least six types of particles that can be formulated with silk fibroin and the active agent: (1) nanoparticles comprising a core formed by silk fibroin to which the active agent absorbs/adsorbs or forms a coating on the nanoparticle core; (2) nanoparticles comprising a core formed by the active agent, which is coated with one or more layers of silk fibroin; (3) nanoparticles comprising a generally homogeneous mixture of silk fibroin and the active agent; (4) nanoparticles comprising a core comprising a mixture of silk fibroin and the active agent with a coating over the core of silk fibroin; (5) a nanoparticle comprising a core of a material other than silk fibroin or active agent, which is coated with one more layers comprising active agent or silk fibroin or any combination of active agent and silk fibroin; and (6) nanoparticle comprising any of the nanoparticles of (1)-(5) and further comprising one or more layers of a material other than silk fibroin or active agent, e.g., a polymer. Silk fibroin particles (e.g., microspheres, nanospheres, or gel like particles) and methods of preparing the same are described, for example, in U.S. Pat. No. 8,187,616; and U.S. Pat. App. Pub. Nos. US 2008/0085272, US 2010/0028451, US 2012/0052124, US 2012/0070427, US 2012/0187591, the content of all of which is incorporated herein by reference. Without limitations, the active agent can be distributed in the silk fibroin matrix of the film, present on a surface of the film, coated by the film, or any combination thereof.

In some embodiments, the silk-based material can be in the form of a foam or a sponge. Methods for preparing silk gels and hydrogels are well known in the art. In some embodiments, the foam or sponge is a patterned foam or sponge, e.g., nanopatterned foam or sponge. Exemplary methods for preparing silk foams and sponges are described in, for example, WO 2004/000915, WO 2004/000255, and WO 2005/012606, content of all of which is incorporated herein by reference in its entirety. Without limitations, the active agent can be distributed in the silk fibroin matrix of the foam or sponge, absorbed on a surface of the foam or sponge, present in a pore of the foam or sponge, or any combination thereof.

In some embodiments, the silk-based material can be in the form of a gel or hydrogel. The term “hydrogel” is used herein to mean a silk-based material which exhibits the ability to swell in water and to retain a significant portion of water within its structure without dissolution. Methods for preparing silk gels and hydrogels are well known in the art. Exemplary methods for preparing silk gels and hydrogels are described in, for example, WO 2005/012606, content of which is incorporated herein by reference in its entirety. Without limitations, the active agent can be distributed in the silk fibroin matrix of gel or hydrogel, absorbed on a surface of the gel or hydrogel or sponge, present in a pore of the gel or hydrogel, or any combination thereof.

In some embodiments, the silk-based material can be in the form of a cylindrical matrix, e.g., a silk tube. The active agent can be present in the lumen of the cylindrical matrix or dispersed in a wall of the cylindrical matrix. The silk tubes can be made using any method known in the art. For example, tubes can be made using molding, dipping, electrospinning, gel spinning, and the like. Gel spinning is described in Lovett et al. (Biomaterials, 29(35):4650-4657 (2008)) and the construction of gel-spun silk tubes is described in PCT application no. PCT/US2009/039870, filed Apr. 8, 2009, content of both of which is incorporated herein by reference in their entirety. Construction of silk tubes using the dip-coating method is described in PCT application no. PCT/US2008/072742, filed Aug. 11, 2008, content of which is incorporated herein by reference in its entirety. Construction of silk tubes using the film-spinning method is described in PCT application No. PCT/US2013/030206, filed Mar. 11, 2013 and US Provisional application No. 61/613,185, filed Mar. 20, 2012. Without wishing to be bound by a theory, it is believed that the inner and outer diameter of the silk tube can be controlled more readily using film-spinning or gel-spinning than dip-coating technique.

In some embodiments, the silk-based material can be porous. For example, the silk-matrix can have a porosity of at least about 10%, at least about 20%, at least about 30%, at least about 40%, at least about 50%, at least about 60%, at least about 70%, at least about 80%, at least about 90%, or higher. Too high porosity can yield a silk matrix with lower mechanical properties, but with faster release of a molecule encapsulated therein. However, too low porosity can decrease the release of a molecule encapsulated in the matrix. One of skill in the art can adjust the porosity accordingly, based on a number of factors such as, but not limited to, desired release rates, molecular size and/or diffusion coefficient of the molecule encapsulated in the matrix, and/or concentrations, amounts of silk fibroin in the silk tube, and/or desired physical or mechanical properties of the matrix. As used herein, the term “porosity” is a measure of void spaces in a material and is a fraction of volume of voids over the total volume, as a percentage between 0 and 100% (or between 0 and 1). Determination of porosity is well known to a skilled artisan, e.g., using standardized techniques, such as mercury porosimetry and gas adsorption, e.g., nitrogen adsorption.

The porous silk-based material can have any pore size. As used herein, the term “pore size” refers to a diameter or an effective diameter of the cross-sections of the pores. The term “pore size” can also refer to an average diameter or an average effective diameter of the cross-sections of the pores, based on the measurements of a plurality of pores. The effective diameter of a cross-section that is not circular equals the diameter of a circular cross-section that has the same cross-sectional area as that of the non-circular cross-section. In some embodiments, the pores of the matrix can have a size distribution ranging from about 50 nm to about 1000 μm, from about 250 nm to about 500 μm, from about 500 nm to about 250 μm, from about 1 μm to about 200 μm, from about 10 μm to about 150 μm, or from about 50 μm to about 100 μm. In some embodiments, the silk matrix can be swellable when hydrated. The sizes of the pores can then change depending on the water content in the silk matrix. In some embodiment, the pores can be filled with a fluid such as water or air.

Methods for forming pores in a silk-based material are known in the art and include, but are not limited, porogen-leaching methods, freeze-drying methods, and/or gas-forming method. Exemplary methods for forming pores in a silk-based material are described, for example, in U.S. Pat. App. Pub. Nos.: US 2010/0279112 and US 2010/0279112; U.S. Pat. No. 7,842,780; and WO2004062697, content of all of which is incorporated herein by reference in its entirety.

Though not meant to be bound by a theory, silk-based material porosity, structure and mechanical properties can be controlled via different post-spinning processes such as vapor annealing, heat treatment, alcohol treatment, air-drying, lyophilization and the like. Additionally, any desirable release rates, profiles or kinetics of a molecule encapsulated in the matrix can be controlled by varying processing parameters, such as matrix thickness, silk molecular weight, concentration of silk in the matrix, beta-sheet conformation structures, silk II beta-sheet crystallinity, or porosity and pore sizes.

In some embodiments, the silk-based material can be in the form of an implant or scaffold, such as a drug delivery reservoir. As used herein the term “implant” includes within its scope any device intended to be implanted into the body of a vertebrate animal, in particular a mammal, such as a human. An implant can be a drug delivery reservoir for a controlled, sustained release of an active agent in a subject.

For incorporating the active agent in a silk-fibroin matrix, the active agent can be included in a silk fibroin solution used for producing the matrix. Alternatively, or in addition, a preformed silk-based material can be added to a solution comprising the active agent and letting the active agent absorb in/on the matrix.

For incorporating into the silk-based material, the active agent can be in any form suitable for the particular method to be used for fabricating the silk-based material. For example, the active agent can be in the form of a solid, liquid, or gel. In some embodiments, the active agent is in the form of a solution, powder, a compressed powder or a pellet. In some embodiments, the active agent can be encapsulated in a silk fibroin particle for incorporating into the silk-based material. The active agent can be encapsulated in a silk matrix, e.g., by blending the therapeutic agent into a silk solution before processing into a desired material state, e.g., a microsphere or a nanosphere for incorporating into the silk-based material disclosed herein. Silk fibroin particles (e.g., microspheres or nanospheres) which encapsulate an active agent are described, for example, in U.S. Pat. No. 8,187,616; and U.S. Pat. App. Pub. Nos. US 2008/0085272, US 2010/0028451, US 2012/0052124, US 2012/0070427, US 2012/0187591, the content of all of which is incorporated herein by reference.

### Silk Fibroin

As used herein, the term “silk fibroin” or “fibroin” includes silkworm fibroin and insect or spider silk protein. See e.g., Lucas et al., 13 Adv. Protein Chem. 107 (1958). Any type of silk fibroin can be used according to aspects of the present invention. Silk fibroin produced by silkworms, such as Bombyx mori, is the most common and represents an earth-friendly, renewable resource. For instance, silk fibroin can be attained by extracting sericin from the cocoons of B. mori. Organic silkworm cocoons are also commercially available. There are many different silks, however, including spider silk (e.g., obtained from Nephila clavipes), transgenic silks, genetically engineered silks (recombinant silk), such as silks from bacteria, yeast, mammalian cells, transgenic animals, or transgenic plants, and variants thereof, that can be used. See for example, WO 97/08315 and U.S. Pat. No. 5,245,012, content of both of which is incorporated herein by reference in its entirety. In some embodiments, silk fibroin can be derived from other sources such as spiders, other silkworms, bees, and bioengineered variants thereof. In some embodiments, silk fibroin can be extracted from a gland of silkworm or transgenic silkworms. See for example, WO2007/098951, content of which is incorporated herein by reference in its entirety. In some embodiments, silk fibroin is free, or essentially free of sericin, i.e., silk fibroin is a substantially sericin-depleted silk fibroin.

In some embodiments, the silk fibroin can include an amphiphilic peptide. In other embodiments, the silk fibroin can exclude an amphiphilic peptide. “Amphiphilic peptides” possess both hydrophilic and hydrophobic properties. Amphiphilic molecules can generally interact with biological membranes by insertion of the hydrophobic part into the lipid membrane, while exposing the hydrophilic part to the aqueous environment. In some embodiment, the amphiphilic peptide can comprise a RGD motif. An example of an amphiphilic peptide is a 23RGD peptide having an amino acid sequence: HOOC-Gly-ArgGly-Asp-Ile-Pro-Ala-Ser-Ser-Lys-Gly-Gly-Gly-Gly-SerArg-Leu-Leu-Leu-Leu-Leu-Leu-Arg-NH2. Other examples of amphiphilic peptides include the ones disclosed in the U.S. Patent App. No.: US 2011/0008406, the content of which is incorporated herein by reference.

The silk fibroin solution can be prepared by any conventional method known to one skilled in the art. For example, B. mori cocoons are boiled for about 30 minutes in an aqueous solution. Preferably, the aqueous solution is about 0.02M Na₂CO₃. The cocoons are rinsed, for example, with water to extract the sericin proteins and the extracted silk is dissolved in an aqueous salt solution. Salts useful for this purpose include lithium bromide, lithium thiocyanate, calcium nitrate or other chemicals capable of solubilizing silk. Preferably, the extracted silk is dissolved in about 9-12 M LiBr solution. The salt is consequently removed using, for example, dialysis or chromatography.

If necessary, the solution can then be concentrated using, for example, dialysis against a hygroscopic polymer, for example, PEG, a polyethylene oxide, amylose or sericin. Preferably, the PEG is of a molecular weight of 8,000-10,000 g/mol and has a concentration of 10-50%. A slide-a-lyzer dialysis cassette (e.g., Pierce, MW CO 3500) is used. However, any dialysis system may be used. The dialysis is for a time period sufficient to result in a final concentration of aqueous silk solution between 10-30%. In most cases dialysis for 2-12 hours is sufficient. See, for example, PCT application PCT/US/04/11199, content of which is incorporated herein by reference.

Alternatively, the silk fibroin solution can be produced using organic solvents. Such methods have been described, for example, in Li, M., et al., J. Appl. Poly Sci. 2001, 79, 2192-2199; Min, S., et al. Sen′I Gakkaishi 1997, 54, 85-92; Nazarov, R. et al., Biomacromolecules 2004 May-Jun;5(3):718-26. Exemplary organic solvents that can be used to produce the silk solution include, but are not limited to, hexafluoroisopropanol (HFIP). See, for example, International Application No. WO2004/000915, content of which is incorporated herein by reference in its entirety.

Without wishing to be bound by a theory, it is believed that molecular weight of silk used for preparing the compositions disclosed herein can have an effect on properties of the composition, such as active agent release kinetics, swelling ratio, degradation, mechanical properties, and the like.

Silk fibroin solution for forming the composition can have any desired silk fibroin concentration, e.g., a silk fibroin concentration of from about 1% to about 50% (w/v). In some embodiments, the silk fibroin solution has a silk fibroin concentration of from about 10% to about 40% or from 15% to about 35% (w/v). In one embodiment, the silk fibroin solution has a silk fibroin concentration of from about 20% to about 30% (w/v). In one embodiment, the silk fibroin solution has a silk fibroin concentration of about 30% (w/v). In some embodiments, the silk fibroin solution has a silk fibroin concentration of about 0.1% to about 30% (w/v), about 0.5% to about 15% (w/v), about 1% to about 8% (w/v), or about 1.5% to about 5% (w/v). In some embodiments, the silk fibroin solution has a silk fibroin concentration of about 5% to about 30% (w/v), about 10% to about 25% (w/v), or about 15 to about 20% (w/v).

The silk fibroin for making the composition can be modified for different applications or desired mechanical or chemical properties of the matrix (e.g., to facilitate formation of a gradient of an additive (e.g., an active agent) in silk fibroin-based materials). One of skill in the art can select appropriate methods to modify silk fibroins, e.g., depending on the side groups of the silk fibroins, desired reactivity of the silk fibroin and/or desired charge density on the silk fibroin. In one embodiment, modification of silk fibroin can use the amino acid side chain chemistry, such as chemical modifications through covalent bonding, or modifications through charge-charge interaction. Exemplary chemical modification methods include, but are not limited to, carbodiimide coupling reaction (see, e.g. U.S. Patent Application. No. US 2007/0212730), diazonium coupling reaction (see, e.g., U.S. Patent Application No. US 2009/0232963), avidin-biotin interaction (see, e.g., International Application No.: WO 2011/011347) and pegylation with a chemically active or activated derivatives of the PEG polymer (see, e.g., International Application No. WO 2010/057142). Silk fibroin can also be modified through gene modification to alter functionalities of the silk protein (see, e.g., International Application No. WO 2011/006133). For instance, the silk fibroin can be genetically modified, which can provide for further modification of the silk such as the inclusion of a fusion polypeptide comprising a fibrous protein domain and a mineralization domain, which can be used to form an organic-inorganic composite. See WO 2006/076711. In some embodiments, the silk fibroin can be genetically modified to be fused with a protein, e.g., a therapeutic protein. Additionally, the silk fibroin-based material can be combined with a chemical, such as glycerol, that, e.g., affects flexibility of the material. See, e.g., WO 2010/042798, Modified Silk films Containing Glycerol. The contents of the aforementioned patent applications are all incorporated herein by reference.

### Flavor Compositions

In some embodiments, the silk particles and compositions described herein can be used in flavor compositions. A flavor composition refers to a composition comprising at least one flavoring substance. As used herein, the terms “flavor” or “flavoring substance” are understood as meaning a substance having a sensory impression of a food or another substance. In some embodiments, flavors or flavoring substances can encompass odor-releasing substances described herein as certain substances can comprise aroma and flavor properties. The flavors or flavoring substances can be incorporated in the second immiscible phase (e.g., oil droplets) of the compositions or the silk particles described herein. The compositions and/or the silk particles described herein can be used to stabilize and/or control release of the flavors of flavoring substances.

In some embodiments, the flavor composition can comprise an additional different flavor (“flavor co-ingredient”) and/or a flavor adjuvant. These components can be incorporated into the second immiscible phase of the compositions and/or silk particles described herein. Examples of flavors for use as the flavor co-ingredient are described in numerous literature references such as S. Arctander, Perfume and Flavour Chemicals, 1969, Montclair, N.J., USA; Flavor Base 2010 from Leffingwell and Associates; Fenaroli's Handbook of Flavor Ingredients, Sixth Edition; or in other works of a similar nature, as well as in the abundant patent literature in the field of flavor (e.g., but not limited to, International App. No. WO 2011/138696, the content of which is incorporated herein by reference) and the skilled flavorist is readily capable of selecting suitable flavor co-ingredients based on his/her general knowledge and according to the intended application or desired organoleptic effect.

Flavor adjuvants are known in the art and can be selected from, for example, without limitation, solvents, binders, diluents, disintegrating agents, lubricants, coloring agents, preservatives, antioxidants, emulsifiers, stabilizers, flavor-enhancers, sweetening agents, anti-caking agents, enzymes, enzyme-containing preparations and the like. Examples of carriers or diluents for flavor or fragrance compounds can be found in, for instance, “Perfume and Flavor Chemicals”, S. Arctander, Ed., Vol. I & II, “Perfume and Flavor Materials of Natural Origin, S. Arctander, 1960; in “Flavorings”, E. Ziegler and H. Ziegler (ed), Wiley-VCH Weinheim, 1998, and “CTFA Cosmetic Ingredient Handbook”.

The flavor composition described herein can be added to a foodstuff or food product in any suitable form, for example as a liquid, as a paste, as a solid or in encapsulated form bound to or coated onto carriers/particles or as a powder. By way of example only, the flavor composition can be added to, for example, but not limited to, powdered soups, instant noodles, dried pesto mixes, dried savory dishes; stable in-dough flavoring for noodles; beverages or foods, for example, beverages such as fruit drink, fruit wine, lactic drink, carbonated drink, refreshing drink, other drink and the like; ices such as ice cream, sherbet, ice candy and the like; Japanese-style and Western-style confectionaries; jams; candies; jellies; gums; breads; luxury drinks such as coffee, cocoa, black tea, oolong tea, green tea and the like; soups such as Japanese-style soup, Western-style soup, Chinese-style soup and the like; condiments; instant drinks or foods; snacks; oral-care compositions such as dentifrice, oral cleaner, mouth wash, troche, chewing gum and the like; and medicines such as external preparation for skin (e.g. poultice or ointment), internal medicine and the like.

The proportions in which the flavor composition can be incorporated into the various aforementioned articles or products vary within a wide range of values. These values are dependent on the nature of the article to be flavored and on the desired organoleptic effect, as well as the nature of the co-ingredients in a given base, when the compounds according to the invention are mixed with flavoring co-ingredients, solvents or additives commonly used in the art. In some embodiments, the concentration of flavoring substance can range from about 0.1 ppm to about 100 ppm.

### Odor-Releasing Compositions

In some embodiments, the silk particles and compositions described herein can be used in odor-releasing compositions. An odor-releasing composition refers to a composition comprising at least one odor-releasing substance as described herein. The odor-releasing substance can be incorporated in the second immiscible phase (e.g., oil droplets) of the compositions or the silk particles described herein. The compositions and/or the silk particles described herein can be used to stabilize and/or control release of the odor-releasing substance. In some embodiments, odor-releasing substances can encompass flavors or flavoring substances described herein as certain substances can comprise aroma and flavor properties.

In some embodiments, the odor-releasing composition is a fragrance composition. In these embodiments, the odor-releasing substance can comprise one or more of various synthetic aromachemicals, natural essential oils (e.g., bergamot oil, galbanum oil, lemon oil, geranium oil, lavender oil, mandarin oil or the like), synthetic essential oils, citrus oils, animal aromachemicals, plant aromachemicals (e.g., flower-based or fruit-based), and any fragrance components known in the art, for example, but not limited to, α-pinene, limonene, cis-3-hexenol, phenylethyl alcohol, styrallyl acetate, eugenol, rose oxide, linalool, benzaldehyde, muscone, Thesaron (a product of Takasago International Corporation), ethyl butyrate, 2-methylbutanoic acid, etc. and any fragrance component as described in, for example, S. Arctander, “Perfume and Flavor Chemicals”, 1969, Montclair, N.J., USA, as well as International Patent Application Nos. WO 2013/064412; WO 2012/126686; WO 2010/061316; WO 2010/082684; WO 2008/004145; WO 2008/026140; WO 2007/054853; WO 2006/043177; WO 2006/030268; WO 2001/093813; and U.S. Pat. No. 6,743,768; and U.S. Pat. App. No. US 2005/0101498, the content of each of which is incorporated herein by reference.

The fragrance compositions described herein can be used as a fragrance component in fragrance products such as perfume, eau de parfum, eau de toilette, cologne, etc.; in skin-care preparation, face washing cream, vanishing cream, cleansing cream, cold cream, massage cream, milky lotion, toilet water, liquid foundation, pack, makeup remover, etc.; in make-up cosmetic, foundation, face powder, pressed powder, talcum powder, lipstick, rouge, lip cream, cheek rouge, eye liner, mascara, eye shadow, eyebrow pencil, eye pack, nail enamel, enamel remover, etc.; in hair cosmetic, pomade, brilliantine, set lotion, hair stick, hair solid, hair oil, hair treatment, hair cream, hair tonic, hair liquid, hair spray, hair growth agent, hair dye, etc.; in suntan cosmetic, suntan product, sunscreen product, etc.; in medicated cosmetic, antiperspirant, after shave lotion and gel, permanent wave agent, medicated soap, medicated shampoo, medicated skin cosmetic, etc.; in hair-care product, shampoo, rinse, rinse-in-shampoo, conditioner, treatment, hair pack, etc.; in soap, toilet soap, bath soap, perfumed soap, transparent soap, synthetic soap, etc.; as body cleaner, body soap, body shampoo, hand soap, etc.; and, in bath preparation, bath preparations (e.g. bath salt, bath tablet and bath liquid), foam bath (e.g. bubble bath), bath oils (e.g. bath perfume and bath capsule), milk bath, bath jelly, bath cube, etc.; in detergent, heavy-duty detergent for clothing, light-duty detergent for clothing, liquid detergent, washing soap, compact detergent, soap powder, etc.; in fabric softener, softener, furniture care, etc.; in cleaning agent, cleanser, house cleaner, toilet cleaner, bath cleaner, glass cleaner, mold remover, cleaner for waste pipe, etc.; in cleaner for kitchen, soap for kitchen, synthetic soap for kitchen, cleaner for dishes, etc.; in bleaching agent, oxidation type bleaching agent (e.g. chlorine-based bleaching agent or oxygen-based bleaching agent), reduction type bleaching agent (e.g. sulfur-based bleaching agent), optical bleaching agent, etc.; in aerosol, spray type, powder spray type, etc.; in deodorant-aromatic, solid type, gel type, liquid type, etc.; in other articles of manufactures, tissue paper, toilet paper, etc.; and in some embodiments of the personal care compositions described herein.

The amount of incorporation of the odor-releasing composition into a product of interest and/or personal care compositions can range from 0.001 to 50% by weight, and more preferably from 0.01 to 20% by weight.

In some embodiments, at least one fixing agent can be added into the fragrance composition. There can be used, for example, but not limited to, ethylene glycol, propylene glycol, dipropylene glycol, glycerine, hexylene glycol, benzyl benzoate, triethyl citrate, diethyl phthalate, Hercolyn, medium chain fatty acid triglyceride, and medium chain fatty acid diglyceride.

### Personal Care Compositions

In some embodiments, the silk particles and compositions described herein can be provided in different types of personal care compositions. In one embodiment, the personal care composition can be formulated to be a hair care composition selected from the group consisting of shampoo, conditioner, anti-dandruff treatments, styling aids, styling conditioner, hair repair or treatment serum, lotion, cream, pomade, and chemical treatments. In another embodiment, the styling aids are selected from the group consisting of spray, mousse, rinse, gel, foam and a combination thereof. In another embodiment, the chemical treatments are selected from the group consisting of permanent waves, relaxers, and permanent, semi-permanent, and temporary color treatments and combinations thereof.

In another embodiment, the personal care composition can be formulated to be a skin care composition selected from the group consisting of moisturizing body wash, body wash, antimicrobial cleanser, skin protectant treatment, body lotion, facial cream, moisturizing cream, facial cleansing emulsion, surfactant-based facial cleanser, facial exfoliating gel, facial toner, exfoliating cream, facial mask, after shave balm and sunscreen.

In another embodiment, the personal care composition can be formulated to be a cosmetic composition selected from the group consisting of eye gel, lipstick, lip gloss, lip balm, mascara, eyeliner, pressed powder formulation, foundation, fragrance and/or solid perfume. In a further embodiment, the cosmetic composition comprises a makeup composition. Makeup compositions include, but are not limited to color cosmetics, such as mascara, lipstick, lip liner, eye shadow, eye liner, rouge, face powder, make up foundation, and nail polish.

In yet another embodiment, the personal care composition can be formulated to be a nail care composition in a form selected from the group consisting of nail enamel, cuticle treatment, nail polish, nail treatment, and polish remover.

In yet another embodiment, the personal care composition can be formulated to be an oral care composition in a form selected from the group consisting of toothpaste, mouth rinse, breath freshener, whitening treatment, and inert carrier substrates.

In yet another embodiments, the personal care composition can comprise an odor-releasing substance/composition (e.g., fragrance composition) and/or flavoring substance/composition, e.g., to provide and/or improve the scent and/or taste of the personal care composition.

The personal care composition can be in any form to suit the need of an application and/or preference of users. For example, the personal care composition can be in the form of an emulsified vehicle, such as a nutrient cream or lotion, a stabilized gel or dispersioning system, such as skin softener, a nutrient emulsion, a nutrient cream, a massage cream, a treatment serum, a liposomal delivery system, a topical facial pack or mask, a surfactant-based cleansing system such as a shampoo or body wash, an aerosolized or sprayed dispersion or emulsion, a hair or skin conditioner, styling aid, or a pigmented product such as makeup in liquid, cream, solid, anhydrous or pencil form.

In some embodiments of various kinds of the personal care composition described herein, the composition can further comprise an active ingredient or an active agent described herein. One skilled in the art will appreciate the various active ingredients or active agents for use in personal care compositions, any of which may be employed herein, see e.g., McCutcheon's Functional Materials, North American and International Editions, (2003), published by MC Publishing Co. For example, the personal care compositions herein can comprise a skin care active ingredient at a level from about 0.0001% to about 20%, by weight of the composition. In another embodiment, the personal care composition comprises a skin care active ingredient from about 0.001% to about 5%, by weight of the composition. In yet another embodiment, the personal care composition comprises a skin care active ingredient from about 0.01% to about 2%, by weight of the composition.

In some embodiments, the silk particles and compositions described herein can be used to stabilize and/or provide a controlled release or sustained release of at least one skin care active ingredient Skin care active ingredients include, but are not limited to, antioxidants, such as tocopheryl and ascorbyl derivatives; retinoids or retinols; essential oils; bioflavinoids, terpenoids, synthetics of biolflavinoids and terpenoids and the like; vitamins and vitamin derivatives; hydroxyl- and polyhydroxy acids and their derivatives, such as AHAs and BHAs and their reaction products; peptides and polypeptides and their derivatives, such as glycopeptides and lipophilized peptides, heat shock proteins and cytokines; enzymes and enzymes inhibitors and their derivatives, such as proteases, MMP inhibitors, catalases, CoEnzyme Q10, glucose oxidase and superoxide dismutase (SOD); amino acids and their derivatives; bacterial, fungal and yeast fermentation products and their derivatives, including mushrooms, algae and seaweed and their derivatives; phytosterols and plant and plant part extracts; phospholipids and their derivatives; anti-dandruff agents, such as zinc pyrithione, and chemical or organic sunscreen agents such as ethylhexyl methoxycinnamate, avobenzone, phenyl benzimidazole sulfonic acid, and/or zinc oxide. Delivery systems comprising the active ingredients are also provided herein.

In addition to the active ingredients noted above, the personal care composition can further comprise a physiologically acceptable carrier or excipient. Specifically, the personal care compositions herein can comprise a safe and effective amount of a dermatologically acceptable carrier, suitable for topical application to the skin or hair within which the essential materials and optional other materials are incorporated to enable the essential materials and optional components to be delivered to the skin or hair at an appropriate concentration. The carrier can thus act as a diluent, dispersant, solvent or the like for the essential components which ensures that they can be applied to and distributed evenly over the selected target at an appropriate concentration.

An effective amount of the silk particles and compositions described herein can also be included in personal care compositions to be applied to keratinous materials such as nails and hair, including but not limited to those useful as hair spray compositions, hair styling compositions, hair shampooing and/or conditioning compositions, compositions applied for the purpose of hair growth regulation and compositions applied to the hair and scalp for the purpose of treating seborrhea, dermatitis and/or dandruff.

An effective amount of the silk particles and compositions described herein may be included in personal care compositions suitable for topical application to the skin, teeth, nails or hair. These compositions can be in the form of creams, lotions, gels, suspensions dispersions, microemulsions, nanodispersions, microspheres, hydrogels, emulsions (e.g., oil-in-water and water-in-oil, as well as multiple emulsions) and multilaminar gels and the like (see, for example, The Chemistry and Manufacture of Cosmetics, Schlossman et al., 1998), and can be formulated as aqueous or silicone compositions or can be formulated as emulsions of one or more oil phases in an aqueous continuous phase (or an aqueous phase in an oil phase).

A variety of optional ingredients such as neutralizing agents, fragrance, perfumes and perfume solubilizing agents, coloring agents, surfactants, emulsifiers, and/or thickening agents can also be added to the personal care compositions herein. Any additional ingredients should enhance the product, for example, the skin softness/smoothness benefits of the product. In addition, any such ingredients should not negatively impact the aesthetic properties of the product.

Suitably, the pH of the personal care compositions herein is in the range from about 3.5 to about 10, specifically from about 4 to about 8, and more specifically from about 5 to about 7, wherein the pH of the final composition is adjusted by addition of acidic, basic or buffer salts as necessary, depending upon the composition of the forms and the pH-requirements of the compounds.

One skilled in the art will appreciate the various techniques for preparing the personal care compositions of the present invention, any of which may be employed herein.

### Pharmaceutical Compositions and Controlled/Sustained Release

The silk particles and/or silk-based composition disclosed herein provide for controlled or sustained release of an active agent from silk-based material, and/or from the silk-immiscible phase (e.g., lipid compartments such as oil). As used herein, the term “sustained delivery” is refers to continual delivery of an active agent in vivo or in vitro over a period of time following administration. For example, sustained release can occur over a period of at least several days, a week or several weeks. Sustained delivery of the agent in vivo can be demonstrated by, for example, the continued therapeutic effect of the agent over time. Alternatively, sustained delivery of the agent can be demonstrated by detecting the presence of the agent in vivo over time. In some embodiments, the sustain release is over a period of one week, two weeks, three weeks, four weeks, one month, two months, three months, four months, five months, six months or longer.

In some embodiments, the silk particles and/or silk-based compositions described herein can be used for drug delivery and provide or release an amount of the active agent, which provides a therapeutic effect similar to as provided by a recommended dosage of the active agent for the same period of time. For example, if the recommended dosage for the active agent is once daily, then the composition releases that amount of active agent, which is sufficient to provide a similar therapeutic effect as provided by the once daily dosage.

Daily release of the active agent can range from about 1 ng/day to about 1000 mg/day. For example, amount released can be in a range with a lower limit of from 1 to 1000 (e.g., every integer from 1 to 1000) and upper limit of from 1 to 1000 (e.g. every integer from 1 to 1000), wherein the lower and upper limit units can be selected independently from ng/day, μg/day, mg/day, or any combinations thereof.

In some embodiments, daily release can be from about 1 μg/day to about 10 mg/day, from about 0.25 μg/day to about 2.5 mg/day, or from about 0.5 μg/day to about 5 mg/day. In some embodiments, daily release of the active agent can range from about 100 ng/day to 1 mg/day, for example, or about 500 ng/day to 5 mg/day, or about 100 μg/day.

In some embodiments, release of the active agent follows near zero-order release kinetics over a period of time. For example, near zero-order release kinetics can be achieved over a period of one week, two weeks, three weeks, four weeks, one month, two months, three months, four months, five months, six months, twelve months, one year or longer.

In some embodiments, no significant apparent initial burst release is observed from the composition described herein. Accordingly, in some embodiments, the initial burst of the active agent within the first 48, 24, 18, 12, or 6 hours of administration of a composition disclosed herein is less than 25%, less than 20%, less than 15%, less than 10%, less than 9%, less than 8%, less than 7%, less than 6%, less than 5%, less than 4%, less than 3%, less than 2%, or less than 1% of the total amount of active agent present in the composition. In some embodiments, there is no noticeable or measurable initial burst of the active agent within the first 6 or 12 hours, 1, 2, 3, 4, 5, 6, 7 days, 1 and 2 weeks of administration.

In yet another aspect, the disclosure provides a method of sustained delivery in vivo of an active agent. The method comprising administering silk particles and/or compositions described herein comprising an active agent as disclosed herein to a subject. Without wishing to be bound by a theory, the active agent can be released in a therapeutically effective amount daily. As used herein, the term “therapeutically effective amount” means an amount of the active agent which is effective to provide a desired outcome. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders. Guidance regarding the efficacy and dosage which will deliver a therapeutically effective amount of a compound can be obtained from animal models of condition to be treated.

As disclosed herein, the silk-based material comprising the active agent can provide a therapeutically effective amount of the active agent to a subject for a period of time which is similar to or longer than the period of time when the active agent is administered without the silk-based material. For example, amount of active agent released over a day provides a similar therapeutic effect as provided by the recommended daily dosage of the active agent when administered without the silk-based material disclosed herein.

For administration to a subject, the silk-based material can be formulated in pharmaceutically acceptable compositions which comprise a silk-based material disclosed herein, formulated together with one or more pharmaceutically acceptable carriers (additives) and/or diluents. The composition can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery composition. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

As used herein, the term “administered” refers to the placement of a drug delivery composition into a subject by a method or route which results in at least partial localization of the pharmaceutically active agent at a desired site. A drug delivery composition described herein can be administered by any appropriate route which results in effective treatment in the subject, i.e. administration results in delivery to a desired location in the subject where at least a portion of the pharmaceutically active agent is delivered. Exemplary modes of administration include, but are not limited to, implant, injection, infusion, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

In some embodiments, the silk-based material disclosed herein can be implanted in a subject. As used herein, the term “implanted,” and grammatically related terms, refers to the positioning of the silk-based material in a particular locus in the subject, either temporarily, semi-permanently, or permanently. The term does not require a permanent fixation of the silk-based material in a particular position or location. Exemplary in vivo loci include, but are not limited to site of a wound, trauma or disease.

### Additional Examples of Additives

In some embodiments, the silk-based material and/or composition can further comprise one or more additives. For example, the composition can be prepared from a fibroin solution comprising one or more (e.g., one, two, three, four, five or more) additives. Without wishing to be bound by theory, additive can provide a silk-based material with desired properties, e.g., provide flexibility, solubility, ease of processing, and the like.

In some embodiments, the second immiscible phase can further comprise one or more additives. For example, the composition can be prepared from a second immiscible solution comprising one or more (e.g., one, two, three, four, five or more) additives. Without wishing to be bound by theory, additive can provide the second immiscible with desired properties, e.g., emulsion stability.

Without limitations, an additive can be selected from small organic or inorganic molecules; saccharides; oligosaccharides; polysaccharides; polymers; proteins; peptides; peptide analogs and derivatives; peptidomimetics; nucleic acids; nucleic acid analogs; and the like. Total amount of additives in the solution can be from about 0.1 wt % to about 70 wt %, from about 5 wt % to about 60 wt %, from about 10 wt % to about 50 wt %, from about 15 wt % to about 45 wt %, or from about 20 wt % to about 40 wt %, of the total silk fibroin in the solution.

In some embodiments, an additive is a biocompatible polymer as described earlier.

In one embodiment, the additive is glycerol, which can affect the flexibility and/or solubility of the silk-based. Silk-based material, e.g., silk films comprising glycerol are described in WO 2010/042798, content of which is incorporated herein by reference in its entirety.

In some embodiments, the additive is a stabilizing agent. As used herein, the term “stabilizing agent” refers to compounds and compositions that can have a stabilizing effect on the active agent and thereby can help in maintaining the bioactivity of the agent. In some embodiments, the stabilizing agent can be a co-factor needed by the active agent for bioactivity.

In some embodiments, the additive can comprise a stimulus-responsive agent. As used herein, the term “stimulus-responsive” means that one or more chemical, physical and/or biological properties can change in response to a stimulus described herein. Depending on the nature and/or properties of the stimulus-responsive agent, various types of responses can occur, including, e.g., but not limited to size change, density change, chemical structural change, conformational change, enzymatic reaction, redox reaction, bond or linkage cleavage/formation, changes in magnetic properties, cytokine production and/or secretion, change in optical properties (e.g., but not limited to, color, and opacity), change in mechanical properties (e.g., but not limited to, flexibility, stiffness, porosity), matrix degradation, signal transmission, heat emission, light emission and any combinations thereof.

In some embodiments, a stimulus-responsive agent that can be encapsulated in a silk-based material comprises a plasmonic particle, or gold nanoparticle, which can emit light and/or heat upon shining with a light of a specific wavelength. In this embodiment, the plasmonic particle or gold nanoparticle can locally generate heart in a silk-based material, e.g., to facilitate the release of an active agent encapsulated therein, and/or degradation of the silk matrix.

### Targeting Ligands

For some embodiments of the silk particles or compositions described herein, the silk-based material can also comprise a targeting ligand. In these embodiments, the silk particles or compositions described herein can be used to target specific cells for delivery of an active agent. As used herein, the term “targeting ligand” refers to any material or substance which can promote targeting of the silk-based composition to cells, organs, tissues and/or receptors in vivo and/or in vitro. The targeting ligand can be synthetic, semi-synthetic, or naturally-occurring. Materials or substances which can serve as targeting ligands include, for example, proteins, including antibodies, antibody fragments, hormones, hormone analogues, glycoproteins and lectins, peptides, polypeptides, amino acids, sugars, saccharides, including monosaccharides and polysaccharides, carbohydrates, vitamins, steroids, steroid analogs, hormones, cofactors, and genetic material, including nucleosides, nucleotides, nucleotide acid constructs, peptide nucleic acids (PNA), aptamers, and polynucleotides. Other targeting ligands in the present disclosure include cell adhesion molecules (CAM), among which are, for example, cytokines, integrins, cadherins, immunoglobulins and selectin. The silk drug delivery composition can also encompass precursor targeting ligands. A precursor to a targeting ligand refers to any material or substance which can be converted to a targeting ligand. Such conversion can involve, for example, anchoring a precursor to a targeting ligand. Exemplary targeting precursor moieties include maleimide groups, disulfide groups, such as ortho-pyridyl disulfide, vinylsulfone groups, azide groups, and [agr]-iodo acetyl groups.

The targeting ligand can be covalently (e.g., cross-linked) or non-covalently linked to the silk-based material. For example, a targeting ligand can be covalently linked to silk fibroin used for making the silk matrix. Alternatively, or in addition, a targeting ligand can be linked to an additive present in the silk fibroin solution which is used for making the silk-based material.

Embodiments of various aspects described herein can be defined in any of the following numbered paragraphs:
1. A silk particle comprising at least two immiscible phases, a first immiscible phase comprising a silk-based material and a second immiscible phase comprising an active agent, wherein the first immiscible phase encapsulates the second immiscible phase and the second immiscible phase excludes a liposome.
2. The silk particle of paragraph 1, wherein the second immiscible phase comprises a lipid component.
3. The silk particle of paragraph 2, wherein the lipid component comprises oil.
4. The silk particle of any of paragraphs 1-3, wherein the second immiscible phase forms a single compartment.
5. The silk particle of any of paragraphs 1-3, wherein the second immiscible phase forms a plurality of compartments.
6. The silk particle of paragraph 4 or 5, wherein the size of the compartment or compartments ranges from about 1 nm to about 1000 μm, or from about 5 nm to about 500 μm.
7. The silk particle of any of paragraphs 1-6, wherein the active agent present in the second immiscible phase comprises a hydrophobic or lipophilic molecule.
8. The silk particle of paragraph 7, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof
9. The silk particle of any of paragraphs 1-8, wherein the silk-based material comprises an additive.
10. The silk particle of paragraph 9, wherein the additive is selected from the group consisting of biocompatible polymers; plasticizers (e.g., glycerol); stimulus-responsive agents; active agents, small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.
11. The silk particle of paragraph 9 or 10, wherein the additive is in a form of a particle (e.g., a nanoparticle or microparticle, including a plasmonic particle), a fiber, a tube, powder or any combinations thereof
12. The silk particle of any of paragraphs 9-11, wherein the additive comprises a silk material, e.g., silk particles, silk fibers, micro-sized silk fibers, unprocessed silk fibers, and any combinations thereof
13. The silk particle of any of paragraphs 1-12, wherein the second immiscible phase encapsulates a third immiscible phase.
14. The silk particle of any of paragraphs 1-13, wherein the silk-based material is present in a form of a hydrogel.
15. The silk particle of any of paragraphs 1-14, wherein the silk-based material is present in a dried state or lyophilized.
16. The silk particle of paragraph 15, wherein the lyophilized silk matrix is porous.
17. The silk particle of any of paragraphs 1-16, wherein the silk-based material in the first immiscible phase is soluble in an aqueous solution.
18. The silk particle of any of paragraphs 1-17, wherein beta-sheet content in the silk-based material is adjusted to an amount sufficient to enable the silk-based material to resist dissolution in an aqueous solution.
19. The silk particle of any of paragraphs 1-18, wherein the size of the silk particle ranges from about 10 nm to about 10 mm, or from about 50 nm to about 5 mm.
20. A composition comprising a plurality of lipid compartments encapsulated in a silk-based material.
21. The composition of paragraph 20, wherein the size of the lipid compartments ranges from about 1 nm to about 1000 μm, or from about 5 nm to about 500 μm.
22. The composition of paragraph 20 or 21, wherein the volumetric ratio of the lipid compartments to the silk-based material ranges from about 1000:1 to about 1:1000, from about 500:1 to about 1:500, or from about 100:1 to about 1:100.
23. The composition of any of paragraphs 20-22, wherein the silk-based material is in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized material, a porous material, a non-porous material, and any combinations thereof.
24. The composition of any of paragraphs 20-23, wherein the silk-based material comprises a film.
25. The composition of any of paragraphs 20-24, wherein the silk-based material comprises a scaffold.
26. The composition of any of paragraphs 20-25, wherein the silk-based material comprises an optical pattern.
27. The composition of paragraph 26, wherein the optical pattern comprises a hologram or an array of patterns that provides an optical functionality.
28. The composition of any of paragraphs 20-27, wherein the lipid compartments further comprise an active agent.
29. The composition of paragraph 20-28, wherein the active agent comprises a hydrophobic or lipophilic molecule.
30. The composition of paragraph 29, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof.
31. The composition of any of paragraphs 20-30, wherein the silk-based material comprises an additive.
32. The composition of paragraph 31, wherein the additive is selected from the group consisting of biocompatible polymers; plasticizers (e.g., glycerol); stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof
33. The composition of paragraph 31 or 32, wherein the additive is in a form selected from the group consisting of a particle, a fiber, a tube, a film, a gel, a mesh, a mat, a non-woven mat, a powder, and any combinations thereof
34. The composition of any of paragraphs 31-33, wherein the additive comprises a silk material, e.g., silk particles, silk fibers, micro-sized silk fibers, unprocessed silk fibers, and any combinations thereof
35. A composition comprising a collection of silk particles of any of paragraphs 1-19.
36. The composition of paragraph 35, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, a scaffold, or any combinations thereof.
37. The composition of paragraph 35 or 36, wherein the composition is formulated for use in a pharmaceutical product.
38. The composition of paragraph 35 or 36, wherein the composition is formulated for use in a cosmetic product.
39. The composition of paragraph 35 or 36, wherein the composition is formulated for use in a personal care product.
40. The composition of paragraph 35 or 36, wherein the composition is formulated for use in a food product.
41. A storage-stable composition comprising a silk particle of any of paragraphs 1-19 or a composition of any of paragraphs 20-40, wherein the active agent present in the second immiscible phase of the silk particle, or a hydrophobic or lipophilic molecule present in the lipid components retains at least about 30% of its original bioactivity after the composition is (a) subjected to at least one freeze-thaw cycle, or (b) maintained for at least about 24 hours at a temperature of about room temperature or above, or (c) both (a) and (b).
42. The composition of paragraph 41, wherein the composition is maintained under exposure to light.
43. The composition of paragraph 41 or 42, wherein the composition is maintained at a relative humidity of at least about 10%.
44. The composition of any of paragraphs 41-43, wherein the silk-based material of the silk particle or the composition is in a dried-state.
45. A method of producing a silk particle comprising:
   a. providing an emulsion of non-aqueous droplets dispersed in a silk solution undergoing a sol-gel transition (where the silk solution remains in a mixable state); and
   b. contacting a pre-determined volume of the emulsion with a non-aqueous phase, whereby the silk solution forms in the non-aqueous phase a silk particle entrapping at least one of the non-aqueous droplets therein.
46. The method of paragraph 45, wherein the sol-gel transition last for about at least 1 hour, or at least about 2 hours.
47. The method of paragraph 45 or 46, wherein the sol-gel transition of the silk solution is induced by sonication.
48. The method of paragraph 47, where the sonication is performed at an amplitude of about 5% to about 20%, or about 10% to about 15%.
49. The method of paragraph 47 or 48, wherein the sonication duration lasts for about 15 sec to about 60 sec, or from about 30 sec to about 45 sec.
50. The method of any of paragraphs 45-49, wherein the silk solution has a concentration of about 1% (w/v) to about 15% (w/v), or about 2% (w/v) to about 7% (w/v).
51. The method of any of paragraphs 45-50, further comprising adding an active agent into the silk fibroin solution undergoing a sol-gel transition.
52. The method of any of paragraphs 45-51, wherein the non-aqueous droplets further comprise a hydrophobic or lipophilic molecule.
53. The method of paragraph 52, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof.
54. The method of any of paragraphs 45-53, wherein the emulsion is produced by adding a non-aqueous, immiscible phase into the silk solution, thereby forming the non-aqueous droplets dispersed in the silk solution.
55. The method of any of paragraphs 45-54, wherein the pre-determined volume of the emulsion substantially corresponds to a desirable size of the silk particle.
56. The method of any of paragraphs 45-55, further comprising isolating the silk particle from the non-aqueous phase.
57. The method of any of paragraphs 45-56, further comprising subjecting the silk particle to a post-treatment.
58. The method of paragraph 57, wherein the post-treatment further induces a conformational change in silk fibroin in the particle.
59. The method of paragraph 58, wherein said inducing conformational change comprises one or more of lyophilization or freeze-drying, water annealing, water vapor annealing, alcohol immersion, sonication, shear stress, electrogelation, pH reduction, salt addition, air-drying, electrospinning, stretching, or any combination thereof.
60. The method of any of paragraphs 57-59, wherein the post-treatment comprises freeze-drying the silk particle.
61. A method comprising a step of: maintaining a composition, wherein the composition comprises at least one lipid compartment encapsulated in a silk-based material and at least one active agent distributed in said at least one lipid compartment, and wherein the active agent retains at least about 30% of its original bioactivity after the composition is (a) subjected to at least one freeze-thaw cycle, or (b) maintained for at least about 24 hours at a temperature of about room temperature or above, or (c) both (a) and (b).
62. The method of paragraph 61, wherein the composition is maintained for at least about 1 month.
63. A method comprising a step of: maintaining a composition, wherein the composition comprises at least one lipid compartment encapsulated in a silk-based material and at least one active agent distributed in said at least one lipid compartment, and wherein the silk-based material is permeable to said at least one active agent such that the active agent is released through the silk-based material into an ambient surrounding at a pre-determined rate.
64. The method of paragraph 63, wherein the pre-determined rate is controlled by adjusting an amount of beta-sheet conformation of silk fibroin present in the silk-based material, porosity of the silk-based material, or a combination thereof.
65. The method of paragraph 63 or 64, wherein the composition is maintained at about room temperature.
66. The method of any of paragraphs 61-65, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.
67. The method of any of paragraphs 61-66, wherein the composition is lyophilized.
68. The method of any of paragraphs 61-67, wherein the composition is maintained at a temperature of about 37° C. or greater.
69. The method of any of paragraphs 61-68, wherein the composition is maintained under exposure to light.
70. The method of any of paragraphs 61-69, wherein the composition is maintained at a relative humidity of at least about 10%.
71. The method of any of paragraphs 61-70, wherein the active agent comprises a hydrophobic or lipophilic active agent.
72. The method of paragraph 71, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof
73. The method of any of paragraphs 61-72, wherein the silk-based material comprises an additive.
74. The method of paragraph 73, wherein the additive is selected from the group consisting of biocompatible polymers; plasticizers (e.g., glycerol); stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.
75. The method of paragraph 73 or 74, wherein the additive is in a form selected from the group consisting of a particle, a fiber, a tube, a film, a gel, a mesh, a mat, a non-woven mat, a powder, and any combinations thereof
76. The method of any of paragraphs 73-75, wherein the additive comprises a silk material, e.g., silk particles, silk fibers, micro-sized silk fibers, unprocessed silk fibers, or any combinations thereof.
77. A method of delivering an active agent comprising applying or administering to a subject a composition comprising a silk-based material, the silk-based material encapsulating at least one lipid compartment with an active agent disposed therein, said silk-based material being permeable to the active agent such that the active agent is released through the silk-based material, at a pre-determined rate, upon application or administration of the composition to the subject.
78. The method of paragraph 77, wherein the active agent is released to an ambient surrounding.
79. The method of paragraph 77 or 78, wherein the active agent is released to at least one target cell of the subject.
80. The method of any of paragraphs 77-79, wherein the active agent comprises a hydrophobic or lipophilic active agent.
81. The method of paragraph 80, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof
82. The method of any of paragraphs 77-81, wherein the silk-based material comprises an additive.
83. The method of any of paragraphs 77-82, wherein the composition is applied or administered to the subject topically.
84. The method of paragraph 83, wherein the composition is applied on a skin of the subject.
85. The method of any of paragraphs 77-82, wherein the composition is applied or administered to the subject orally.
86. A silk particle comprising at least two immiscible phases, a first immiscible phase comprising a silk-based material and a second immiscible phase comprising an active agent, wherein the first immiscible phase encapsulates the second immiscible phase and the second immiscible phase excludes a liposome.
87. The silk particle of paragraph 86, wherein the second immiscible phase comprises a lipid component.
88. The silk particle of paragraph 87, wherein the lipid component comprises oil.
89. The silk particle of any of paragraphs 86-88, wherein the second immiscible phase forms a single compartment.
90. The silk particle of any of paragraphs 86-89, wherein the second immiscible phase forms a plurality of compartments.
91. The silk particle of paragraph 89 or 90, wherein the size of the compartment or compartments ranges from about 1 μm to about 1000 μm, or from about 10 μm to about 500 μm.
92. The silk particle of any of paragraphs 86-91, wherein the active agent present in the second immiscible phase comprises a hydrophobic or lipophilic molecule.
93. The silk particle of paragraph 92, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.
94. The silk particle of any of paragraphs 86-93, wherein the silk-based material comprises an additive.
95. The silk particle of paragraph 94, wherein the additive comprises a biopolymer, an active agent, a plasmonic particle, glycerol, and any combinations thereof.
96. The silk particle of any of paragraphs 86-95, wherein the second immiscible phase encapsulates a third immiscible phase.
97. The silk particle of any of paragraphs 86-96, wherein the silk-based material is present in a form of a hydrogel.
98. The silk particle of any of paragraphs 86-96, wherein the silk-based material is present in a dried state or lyophilized.
99. The silk particle of paragraph 98, wherein the lyophilized silk matrix is porous.
100. The silk particle of any of paragraphs 86-99, wherein at least the silk-based material in the first immiscible phase is soluble in an aqueous solution.
101. The silk particle of any of paragraphs 86-99, wherein beta-sheet content in the silk-based material is adjusted to an amount sufficient to enable the silk-based material to resist dissolution in an aqueous solution.
102. The silk particle of any of paragraphs 86-101, wherein the size of the silk particle ranges from about 0.1 mm to about 10 mm, or from about 0.5 mm to about 5 mm.
103. A composition comprising a plurality of lipid compartments encapsulated in a silk-based material.
104. The composition of paragraph 103, wherein the size of the lipid compartments ranges from about 1 μm to about 1000 μm, or from about 10 μm to about 500 μm.
105. The composition of paragraph 103 or 104, wherein the volumetric ratio of the lipid compartments to the silk-based material ranges from about 1:1 to about 1:1000, from about 1:5 to about 1:500, or from about 1:10 to about 1:100.
106. The composition of any of paragraphs 103-105, wherein the silk-based material comprises a film.
107. The composition of paragraph 106, wherein the silk-based material comprises an optical pattern.
108. The composition of paragraph 107, wherein the optical pattern comprises a hologram or an array of patterns that provides an optical functionality.
109. The composition of any of paragraphs 103-108, wherein the silk-based material comprises a scaffold.
110. The composition of any of paragraphs 103-109, wherein the lipid compartments further comprise an active agent.
111. The composition of paragraph 110, wherein the active agent comprises a hydrophobic or lipophilic molecule.
112. The composition of paragraph 111, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.
113. The composition of any of paragraphs 103-112, wherein the silk-based material comprises an additive.
114. The composition of paragraph 113, wherein the additive comprises a biopolymer, an active agent, a plasmonic particle, glycerol, and any combinations thereof
115. A composition comprising a collection of silk particles of any of paragraphs 86-102.
116. The composition of paragraph 115, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.
117. The composition of paragraph 115 or 116, wherein the composition is formulated for use in a pharmaceutical product.
118. The composition of paragraph 115 or 116, wherein the composition is formulated for use in a cosmetic product.
119. The composition of paragraph 115 or 116, wherein the composition is formulated for use in a food product.
120. A storage-stable composition comprising a silk particle of any of paragraphs 86-102 or a composition of any of paragraphs 103-119, where the active agent present in the second immiscible phase of the silk particle, or a hydrophobic or lipophilic molecule present in the lipid components retains at least about 30% of its original bioactivity when the composition is (a) subjected to at least one freeze-thaw cycle, or (b) maintained for at least about 24 hours at a temperature of about room temperature or above, or (c) both (a) and (b).
121. The composition of paragraph 120, wherein the composition is maintained under exposure to light.
122. The composition of paragraph 120 or 121, wherein the composition is maintained at a relative humidity of at least about 10%.
123. The composition of any of paragraphs 120-122, wherein the cross-linked silk matrix is in a dried-state.
124. A method of producing a silk particle comprising:
a. providing or obtaining an emulsion of non-aqueous droplets dispersed in a silk solution undergoing a sol-gel transition (where the silk solution remains in a mixable state); and
b. contacting a pre-determined volume of the emulsion with a non-aqueous phase, whereby the silk solution entraps at least one of the non-aqueous droplets and gels to form a silk particle dispersed in the non-aqueous phase.
125. The method of paragraph 124, wherein the sol-gel transition last for about at least 1 hour, or at least about 2 hours.
126. The method of paragraph 124 or 125, wherein the sol-gel transition of the silk solution is induced by sonication.
127. The method of paragraph 126, where the sonication is performed at an amplitude of about 5% to about 20%, or about 10% to about 15%.
128. The method of paragraph 126 or 127, wherein the sonication duration lasts for about 15 sec to about 60 sec, or from about 30 sec to about 45 sec.
129. The method of any of paragraphs 124-128, wherein the silk solution has a concentration of about 1% (w/v) to about 15% (w/v), or about 2% (w/v) to about 7% (w/v).
130. The method of any of paragraphs 124-129, further comprising adding an active agent into the silk fibroin solution undergoing a sol-gel transition.
131. The method of any of paragraphs 124-130, wherein the non-aqueous droplets further comprise a hydrophobic or lipophilic molecule.
132. The method of paragraph 131, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.
133. The method of any of paragraphs 124-132, wherein the emulsion is produced by adding a non-aqueous, immiscible phase into the silk solution, thereby forming the non-aqueous droplets dispersed in the silk solution.
134. The method of any of paragraphs 124-133, wherein the pre-determined volume of the emulsion is a volume corresponding to a desirable size of the silk particle.
135. The method of any of paragraphs 124-134, further comprising isolating the silk particle from the non-aqueous phase.
136. The method of any of paragraphs 124-135, further comprising freeze-drying the silk particle.
137. A method comprising a step of: maintaining a composition, wherein the composition comprises at least one lipid compartment encapsulated a silk-based material and at least one active agent distributed in said at least one lipid compartment, and wherein the active agent retains at least about 30% of its original bioactivity when the composition is (a) subjected to at least one freeze-thaw cycle, or (b) maintained for at least about 24 hours at a temperature of about room temperature or above, or (c) both (a) and (b).
138. The method of paragraph 137, wherein the composition is maintained for at least about 1 month.
139. A method comprising a step of: maintaining a composition, wherein the composition comprises at least one lipid compartment encapsulated a silk-based material and at least one active agent distributed in said at least one lipid compartment, and wherein the silk-based material is permeable to said at least one active agent such that the active agent is released through the silk-based material into an ambient surrounding at a pre-determined rate.
140. The method of paragraph 139, wherein the pre-determined rate is controlled by adjusting an amount of beta-sheet conformation of silk fibroin present in the silk-based material, porosity of the silk-based material, or a combination thereof.
141. The method of paragraph 139 or 140, wherein the composition is maintained at about room temperature.
142. The method of any of paragraphs 137-141, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.
143. The method of any of paragraphs 137-142, wherein the composition is lyophilized.
144. The method of any of paragraphs 137-143, wherein the composition is maintained at a temperature of about 37° C. or greater.
145. The method of any of paragraphs 137-144, wherein the composition is maintained under exposure to light.
146. The method of any of paragraphs 137-145, wherein the composition is maintained at a relative humidity of at least about 10%.
147. The method of any of paragraphs 137-146, wherein the active agent comprises a hydrophobic or lipophilic active agent.
148. The method of paragraph 147, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.
149. The method of any of paragraphs 137-148, wherein the silk-based material comprises an additive.
150. The method of paragraph 149, wherein the additive comprises a biopolymer, an active agent, a plasmonic particle, glycerol, and any combinations thereof.
151. A method of delivering an active agent comprising applying or administering to a subject a composition comprising a silk-based material, the silk-based material encapsulating a lipid compartment with an active agent disposed therein, said silk-based material being permeable to the active agent such that the active agent is released through the silk-based material, at a pre-determined rate, upon application or administration of the composition to the subject.
152. The method of paragraph 151, wherein the active agent is released to an ambient surrounding.
153. The method of paragraph 151 or 152, wherein the active agent is released to at least one target cell of the subject.
154. The method of any of paragraphs 151-153, wherein the active agent comprises a hydrophobic or lipophilic active agent.
155. The method of paragraph 154, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.
156. The method of any of paragraphs 151-155, wherein the silk-based material comprises an additive.
157. The method of paragraph 156, wherein the additive comprises a biopolymer, an active agent, a plasmonic particle, glycerol, and any combinations thereof.
158. The method of any of paragraphs 151-157, wherein the composition is applied or administered to the subject topically or orally.
159. The method of any of paragraphs 151-158, wherein the composition is applied on skin of the subject.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The term “tube” here refers to an elongated shaft with a lumen therein. The tube can typically be an elongate hollow cylinder, but may also be a hollow shaft of other cross-sectional shapes.

The term “a plurality of” as used herein refers to 2 or more, including, e.g., 3 or more, 4 or more, 5 or more, 6 or more, 7 or more, 8 or more, 9 or more, 10 or more, 20 or more, 30 or more, 40 or more, 50 or more, 100 or more, 500 or more, 1000 or more, 5000 or more, or 10000 or more.

As used herein, a “subject” means a living subject or a physical non-living object, e.g., an article of manufacture. In some embodiments, a subject is a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used interchangeably herein, the terms “essentially” and “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “essentially” means a proportion of at least about 90%, at least about 95%, at least about 98%, at least about 99% or more, or any integer between 90% and 100%. In some embodiments, the term “essentially” can include 100%.

The term “nanopattern” or “nanopatterned” as used herein refers to small patterning that is provided in a silk fibroin-based matrix, e.g., film or foam, or compositions comprising such a silk fibroin-based matrix. Generally, the patterning having structural features of a size that can be appropriately measured in a nanometer scale (i.e., 10⁻⁹ meters), for instance, sizes ranging from 1 nanometer to millimeters, inclusive.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

As used herein, the term “nucleic acid” or “oligonucleotide” or grammatical equivalents herein means at least two nucleotides, including analogs or derivatives thereof, that are covalently linked together. Exemplary oligonucleotides include, but are not limited to, single-stranded and double-stranded siRNAs and other RNA interference reagents (RNAi agents or iRNA agents), shRNA (short hairpin RNAs), antisense oligonucleotides, aptamers, ribozymes, and microRNAs (miRNAs). The nucleic acids can be single stranded or double stranded. The nucleic acid can be DNA, RNA or a hybrid, where the nucleic acid contains any combination of deoxyribo- and ribo-nucleotides, and any combination of uracil, adenine, thymine, cytosine and guanine. The nucleic acids can comprise one or more backbone modifications, e.g., phosphoramide (Beaucage et al., Tetrahedron 49(10):1925 (1993) and references therein; Letsinger, J. Org. Chem. 35:3800 (1970)), phosphorothioate, phosphorodithioate, O-methylphophoroamidite linkages (see Eckstein, Oligonucleotides and Analogues: A Practical Approach, Oxford University Press), or peptide nucleic acid linkages (see Egholm, J. Am. Chem. Soc. 114:1895 (1992); Meier et al., Chem. Int. Ed. Engl. 31:1008 (1992); and Nielsen, Nature, 365:566 (1993), content of all of which is herein incorporated by reference. The nucleic acids can also include modifications to nucleobase and/or sugar moieties of nucleotides. Exemplary sugar modifications at the sugar moiety include replacement of 2′-OH with halogens (e.g., fluoro), O-mehtyl, O-methoxyethyl, NH₂, SH and S-methyl. The term “nucleic acid” also encompasses modified RNA (modRNA). The term “nucleic acid” also encompasses siRNA, shRNA, or any combinations thereof.

The term “modified RNA” means that at least a portion of the RNA has been modified, e.g., in its ribose unit, in its nitrogenous base, in its internucleoside linkage group, or any combinations thereof. Accordingly, in some embodiments, a “modified RNA” may contain a sugar moiety which differs from ribose, such as a ribose monomer where the 2′-OH group has been modified. Alternatively, or in addition to being modified at its ribose unit, a “modified RNA” may contain a nitrogenous base which differs from A, C, G and U (a “non-RNA nucleobase”), such as T or MeC. In some embodiments, a “modified RNA” may contain an internucleoside linkage group which is different from phosphate (—O—P(O)2-O—), such as —O—P(O,S)—O—. In some embodiments, a modified RNA can encompass locked nucleic acid (LNA).

As used herein, the term “polysaccharide” refers to macromolecular carbohydrates whose molecule consists of a large number of monosaccharide molecules which are joined to one another by glycosidic linkage. The term polysaccharide is also intended to embrace an oligosaccharide. The polysaccharide can be homopolysaccharides or heteropolysaccharides. Whereas the homopolysaccharides contain only one kind of unit, the heteropolysaccharides consist of monomer units of different kinds.

The term “short interfering RNA” (siRNA), also referred to herein as “small interfering RNA” is defined as an agent which functions to inhibit expression of a target gene, e.g., by RNAi. An siRNA can be chemically synthesized, it can be produced by in vitro transcription, or it can be produced within a host cell. siRNA molecules can also be generated by cleavage of double stranded RNA, where one strand is identical to the message to be inactivated. The term “siRNA” refers to small inhibitory RNA duplexes that induce the RNA interference (RNAi) pathway. These molecules can vary in length (generally 18-30 base pairs) and contain varying degrees of complementarity to their target mRNA in the antisense strand. Some, but not all, siRNA have unpaired overhanging bases on the 5′ or 3′ end of the sense 60 strand and/or the antisense strand. The term “siRNA” includes duplexes of two separate strands, as well as single strands that can form hairpin structures comprising a duplex region.

The term “shRNA” as used herein refers to short hairpin RNA which functions as RNAi and/or siRNA species but differs in that shRNA species are double stranded hairpin-like structure for increased stability. The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA.

The term “enzymes” as used here refers to a protein molecule that catalyzes chemical reactions of other substances without it being destroyed or substantially altered upon completion of the reactions. The term can include naturally occurring enzymes and bioengineered enzymes or mixtures thereof. Examples of enzyme families include kinases, dehydrogenases, oxidoreductases, GTPases, carboxyl transferases, acyl transferases, decarboxylases, transaminases, racemases, methyl transferases, formyl transferases, and α-ketodecarboxylases.

The term “vaccines” as used herein refers to any preparation of killed microorganisms, live attenuated organisms, subunit antigens, toxoid antigens, conjugate antigens or other type of antigenic molecule that when introduced into a subjects body produces immunity to a specific disease by causing the activation of the immune system, antibody formation, and/or creating of a T-cell and/or B-cell response. Generally vaccines against microorganisms are directed toward at least part of a virus, bacteria, parasite, mycoplasma, or other infectious agent.

As used herein, the term “aptamers” means a single-stranded, partially single-stranded, partially double-stranded or double-stranded nucleotide sequence capable of specifically recognizing a selected non-oligonucleotide molecule or group of molecules. In some embodiments, the aptamer recognizes the non-oligonucleotide molecule or group of molecules by a mechanism other than Watson-Crick base pairing or triplex formation. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. Methods for selecting aptamers for binding to a molecule are widely known in the art and easily accessible to one of ordinary skill in the art.

As used herein, the term “antibody” or “antibodies” refers to an intact immunoglobulin or to a monoclonal or polyclonal antigen-binding fragment with the Fc (crystallizable fragment) region or FcRn binding fragment of the Fc region. The term “antibodies” also includes “antibody-like molecules”, such as fragments of the antibodies, e.g., antigen-binding fragments. Antigen-binding fragments can be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding fragments specific for various antigens are available commercially from vendors such as R&D Systems, BD Biosciences, e-Biosciences and Miltenyi, or can be raised against these cell-surface markers by methods known to those skilled in the art.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The expression “single-chain Fv” or “scFv” antibody fragments, as used herein, is intended to mean antibody fragments that comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. (The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (VH) Connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. (EP 404,097; WO 93/11161; Hollinger et ah, Proc. Natl. Acad. Sd. USA, P0:6444-6448 (1993)).

In reference to an antibody, the term “bioactivity” includes, but is not limited to, epitope or antigen binding affinity, the in vivo and/or in vitro stability of the antibody, the immunogenic properties of the antibody, e.g., when administered to a human subject, and/or the ability to neutralize or antagonize the bioactivity of a target molecule in vivo or in vitro. The aforementioned properties or characteristics can be observed or measured using art-recognized techniques including, but not limited to, scintillation proximity assays, ELISA, ORIGEN immunoassay (IGEN), fluorescence quenching, fluorescence ELISA, competitive ELISA, SPR analysis including, but not limited to, SPR analysis using a BIAcore biosenser, in vitro and in vivo neutralization assays (see, for example, International Publication No. WO 2006/062685), receptor binding, and immunohistochemistry with tissue sections from different sources including human, primate, or any other source as needed. In reference to an immunogen, the “bioactivity” includes immunogenicity, the definition of which is discussed in detail later. In reference to a virus, the “bioactivity” includes infectivity, the definition of which is discussed in detail later. In reference to a contrast agent, e.g., a dye, the “bioactivity” refers to the ability of a contrast agent when administered to a subject to enhance the contrast of structures or fluids within the subject's body. The bioactivity of a contrast agent also includes, but is not limited to, its ability to interact with a biological environment and/or influence the response of another molecule under certain conditions.

As used herein, the term “small molecules” refers to natural or synthetic molecules including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “cells” used herein refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoietic, neural, stromal, muscle, cardiovascular, hepatic, pulmonary, gastrointestinal stem cells, etc. Yeast cells can also be used as cells in some embodiments. In some embodiments, the cells can be ex vivo or cultured cells, e.g. in vitro. For example, for ex vivo cells, cells can be obtained from a subject, where the subject is healthy and/or affected with a disease. Cells can be obtained, as a non-limiting example, by biopsy or other surgical means know to those skilled in the art.

As used herein, the term “viral vector” typically includes foreign DNA which is desired to be inserted in a host cell and usually includes an expression cassette. The foreign DNA can comprise an entire transcription unit, promoter gene-poly A or the vector can be engineered to contain promoter/transcription termination sequences such that only the gene of interest need be inserted. These types of control sequences are known in the art and include promoters for transcription initiation, optionally with an operator along with ribosome binding site sequences. Viral vectors include, but are not limited to, lentivirus vectors, retroviral vectors, lentiviral vectors, herpes simplex viral vectors, adenoviral vectors, adeno-associated viral (AAV) vectors, EPV, EBV or variants or derivatives thereof. Various companies produce such viral vectors commercially, including, but not limited to, Avigen, Inc. (Alameda, Calif.; AAV vectors), Cell Genesys (Foster City, Calif.; retroviral, adenoviral, AAV, and lentiviral vectors), Clontech (retroviral and baculoviral vectors), Genovo, Inc. (Sharon Hill, Pa.; adenoviral and AAV vectors), Genvec (France; adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g. Adenoviruses, Herpesviruses, Poxviruses), ssDNA viruses (e.g. Parvoviruses), dsRNA viruses (e.g. Reoviruses), (+)ssRNA viruses (e.g. Picornaviruses, Togaviruses), (−)ssRNA viruses (e.g. Orthomyxoviruses, Rhabdoviruses), ssRNA-RT viruses, i.e., (+)sense RNA with DNA intermediate in life-cycle (e.g. Retroviruses), and dsDNA-RT viruses (e.g. Hepadnaviruses). In some embodiments, viruses can also include wild-type (natural) viruses, killed viruses, live attenuated viruses, modified viruses, recombinant viruses or any combinations thereof. Other examples of viruses include, but are not limited to, enveloped viruses, respiratory syncytial viruses, non-enveloped viruses, bacteriophages, recombinant viruses, and viral vectors. The term “bacteriophages” as used herein refers to viruses that infect bacteria.

The term “bacteria” as used herein is intended to encompass all variants of bacteria, for example, prokaryotic organisms and cyanobacteria. Bacteria are small (typical linear dimensions of around 1 m), non-compartmentalized, with circular DNA and ribosomes of 70S.

The term “antibiotics” is used herein to describe a compound or composition which decreases the viability of a microorganism, or which inhibits the growth or reproduction of a microorganism. As used in this disclosure, an antibiotic is further intended to include an antimicrobial, bacteriostatic, or bactericidal agent. Exemplary antibiotics include, but are not limited to, penicillins, cephalosporins, penems, carbapenems, monobactams, aminoglycosides, sulfonamides, macrolides, tetracyclines, lincosides, quinolones, chloramphenicol, vancomycin, metronidazole, rifampin, isoniazid, spectinomycin, trimethoprim, sulfamethoxazole, and the like.

As used herein, the term “antigens” refers to a molecule or a portion of a molecule capable of being bound by a selective binding agent, such as an antibody, and additionally capable of being used in an animal to elicit the production of antibodies capable of binding to an epitope of that antigen. An antigen may have one or more epitopes. The term “antigen” can also refer to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

The term “immunogen” refers to any substance, e.g., vaccines, capable of eliciting an immune response in an organism. An “immunogen” is capable of inducing an immunological response against itself on administration to a subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T-cells that are directed towards the immunogen. A cellular immune response is elicited by the presentation of polypeptide epitopes in association with Class I or Class II MHC molecules to activate antigen-specific CD4+ T helper cells and/or CD8+ cytotoxic T cells. Such a response can also involve activation of monocytes, macrophages, NK cells, basophils, dendritic cells, astrocytes, microglia cells, eosinophils or other components of innate immunity.

As used herein, the term “pro-drug” refers to compounds that can be converted via some chemical or physiological process (e.g., enzymatic processes and metabolic hydrolysis) to an active form. Thus, the term “pro-drug” also refers to a precursor of a biologically active compound that is pharmaceutically acceptable. A pro-drug can be inactive when administered to a subject, but is converted in vivo to an active compound, for example, by hydrolysis to the free carboxylic acid or free hydroxyl. The pro-drug compound often offers advantages of solubility, tissue compatibility or delayed release in an organism. The term “pro-drug” is also meant to include any covalently bonded carriers, which release the active compound in vivo when such pro-drug is administered to a subject. Pro-drugs of an active compound, as described herein, can be prepared by modifying functional groups present in the active compound in such a way that the modifications are cleaved, either in routine manipulation or in vivo, to the parent active compound. Pro-drugs include compounds wherein a hydroxy, amino or mercapto group is bonded to any group that, when the pro-drug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group can be administered as an amide, e.g., acetamide, formamide and benzamide that is converted by hydrolysis in vivo to the amine compound. See Harper, “Drug Latentiation” in Jucker, ed. Progress in Drug Research 4:221-294 (1962); Morozowich et al, “Application of Physical Organic Principles to Pro-drug Design” in E. B. Roche ed. Design of Biopharmaceutical Properties through Pro-drugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Pro-drugs, H. Bundgaard, Elsevier (1985); Wang et al. “Pro-drug approaches to the improved delivery of peptide drug” in Curr. Pharm. Design. 5(4):265-287 (1999); Pauletti et al. (1997) Improvement in peptide bioavailability: Peptidomimetics and Pro-drug Strategies, Adv. Drug. Delivery Rev. 27:235-256; Mizen et al. (1998) “The Use of Esters as Pro-drugs for Oral Delivery of (3-Lactam antibiotics,” Pharm. Biotech. 11:345-365; Gaignault et al. (1996) “Designing Pro-drugs and Bioprecursors I. Carrier Pro-drugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Pro-drugs for the improvement of drug absorption via different routes of administration”, Eur. J. Drug Metab. Pharmacokinet, 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the pro-drug approach”, Controlled Drug Delivery 17: 179-96 (1987); Bundgaard H. “Pro-drugs as a means to improve the delivery of peptide drugs”, Arfv. Drug Delivery Rev. 8(1): 1-38 (1992); Fleisher et al. “Improved oral drug delivery: solubility limitations overcome by the use of pro-drugs”, Arfv. Drug Delivery Rev. 19(2): 115-130 (1996); Fleisher et al. “Design of pro-drugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Pro-drugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Pro-drugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Pro-drugs of anticancer agents”, Adv. Drug Delivery Rev. 19(2): 241-273 (1996); Stella et al., “Pro-drugs. Do they have advantages in clinical practice?”, Drugs 29(5): 455-73 (1985); Tan et al. “Development and optimization of anti-HIV nucleoside analogs and pro-drugs: A review of their cellular pharmacology, structure-activity relationships and pharmacokinetics”, Adv. Drug Delivery Rev. 39(1-3): 117-151 (1999); Taylor, “Improved passive oral drug delivery via pro-drugs”, Adv. Drug Delivery Rev., 19(2): 131-148 (1996); Valentino and Borchardt, “Pro-drug strategies to enhance the intestinal absorption of peptides”, Drug Discovery Today 2(4): 148-155 (1997); Wiebe and Knaus, “Concepts for the design of anti-HIV nucleoside pro-drugs for treating cephalic HIV infection”, Adv. Drug Delivery Rev.: 39(1-3):63-80 (1999); Waller et al., “Pro-drugs”, Br. J. Clin. Pharmac. 28: 497-507 (1989), content of all of which are herein incorporated by reference in its entirety.

The term “aliphatic compound”, as used herein, means a compound having at least one straight-chain, branched or cyclic C1-C12 hydrocarbons which are completely saturated or which contain one or more units of unsaturation, but which are not aromatic. For example, suitable aliphatic groups include substituted or unsubstituted linear, branched or cyclic alkyl, alkenyl, alkynyl groups and hybrids thereof, such as cycloalkyl, (cycloalkyl)alkyl, (cycloalkenyl) alkyl or (cycloalkyl)-alkenyl. In various embodiments, the aliphatic group has one to fifty, one to twenty, one to ten, one to eight, one to six, one to four, or one, two, or three carbons.

The terms “alkyl” (or used interchangeably herein with “alkane” if referenced to a compound), “alkenyl” (or used interchangeably herein with “alkene” if referenced to a compound), and “alkynyl” (or used interchangeably herein with “alkyne” if referenced to a compound), used alone or as part of a larger moiety, refer to a straight and branched chain aliphatic group having from one to fifty or one to twenty, or one to twelve carbon atoms.

As used herein, the term “alkyl” will be used when the carbon atom attaching the aliphatic group to the rest of the molecule is a saturated carbon atom. However, an alkyl group can include unsaturation at other carbon atoms. Thus, alkyl groups include, without limitation, methyl, ethyl, propyl, allyl, propargyl, butyl, pentyl, and hexyl.

As used herein, the term “alkenyl” will be used when the carbon atom attaching the aliphatic group to the rest of the molecule forms part of a carboncarbon double bond. Alkenyl groups include, without limitation, vinyl, 1-propenyl, 1-butenyl, 1-pentenyl, and 1-hexenyl.

As used herein, the term “alkynyl” will be used when the carbon atom attaching the aliphatic group to the rest of the molecule forms part of a carbon-carbon triple bond. Alkynyl groups include, without limitation, ethynyl, 1-propynyl, 1-butynyl, 1-pentynyl, and 1-hexynyl.

The term “cycloaliphatic compound” refers to a compound having at least one saturated or partially unsaturated cyclic aliphatic ring system having from 3 to about 14 members, wherein the aliphatic ring system is optionally substituted. In some embodiments, the cycloaliphatic is a monocyclic hydrocarbon having 3-8 or 3-6 ring carbon atoms. Nonlimiting examples include cyclo-alkane, cyclo-alkene and cyclo-alkyne, cyclopropyl, cyclobutyl, cyclopentyl, cyclopentenyl, cyclohexyl, cyclohexenyl, cycloheptyl, cycloheptenyl, cyclooctyl, cyclooctenyl, and cyclooctadienyl. In some embodiments, the cycloaliphatic is a bridged or fused bicyclic hydrocarbon having 6-12, 6-10, or 6-8 ring carbon atoms, wherein any individual ring in the bicyclic ring system has 3-8-members. In some embodiments, two adjacent substituents on a cycloaliphatic ring, taken together with the intervening ring atoms, form an optionally substituted fused 5- to 6-membered aromatic or 3- to 8-membered non-aromatic ring having 0-3 ring heteroatoms selected from the group consisting of 0, N, and S. Thus, the term “cycloaliphatic” includes aliphatic rings that are fused to one or more aryl, heteroaryl, or heterocyclyl rings, where the radical or point of attachment is on the aliphatic ring. Nonlimiting examples include indanyl, 5,6,7,8-tetrahydroquinoxalinyl, decahydronaphthyl, or tetrahydronaphthyl, where the radical orpoint of attachment is on the aliphatic ring.

The terms “aryl” and “ar-”, used alone or as part of a larger moiety, e.g., “aralkyl”, “aralkoxy”, or “aryloxyalkyl”, refer to a C6 to C14 aromatic hydrocarbon, comprising one to three rings, each of which is optionally substituted. Aryl groups include, without limitation, phenyl, naphthyl, and anthracenyl.

As used herein, the term “aromatic compound” refer to a compound having an optionally substituted mono-, bi-, or tricyclic group having 0-6, preferably 0-4 ring heteroatoms, and having 6, 10, or 14π electrons shared in a cyclic array. Thus, the terms “aromatic compound” encompass a compound having aryl and/or heteroaryl groups.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

### Exemplary Methods for Encapsulation Oil in Silk Fibroin Biomaterials and Compositions Resulted Therefrom

Though many materials have been proposed for encapsulation in various applications, e.g., food, cosmetic and medicinal applications, silk fibroin is an especially attractive encapsulant material due to its unique array of chemical and physical properties. Silk fibroin is a biologically-derived protein polymer purified from the domesticated silkworm (Bombyx mori) cocoons that is FDA-approved, edible (Baycin et al., 2007; Hanawa et al., 1995), non-toxic and relatively inexpensive (Qian et al., 1996). Silk exhibits desirable mechanical properties, biocompatibility (Leal-Egaña and Scheibel, 2010; Meinel et al., 2005; Panilaitis et al., 2003) and biodegrades to non-toxic products via proteolysis (Wang et al., 2008a; Horan et al., 2005). Fibroin has been previously discussed to be used in cosmetics, food and the chemical industry (Bayraktar et al., 2005) and has recently been discussed as a scaffold for tissue engineering (Wang et al., 2006, Altman et al., 2003) and a drug carrier for controlled release (Numata and Kaplan, 2010; Pritchard et al., 2011; Wenk et al., 2011).

While other encapsulation approaches require processing conditions which can potentially degrade delicate compounds and/or compromise the safety of the final product (such as exposure to high heat or the use of toxic cross-linking chemicals (Liu et al., 1996; Qian et al., 1997; Demura et al., 1989; Lu et al., 2010)), stable silk biomaterials can be prepared using mild, ambient, aqueous processing conditions (Numata and Kaplan, 2010; Pritchard and Kaplan, 2011). In particular, silk self-assembly into films occurs during drying at ambient conditions of temperature and pressure (Hofmann et al., 2006) and physically cross-linked beta-sheet rich silk hydrogels have been prepared using sonication (Wang et al., 2008b).

Unlike many biologically derived proteins, silk is inherently stable to changes in temperature, pH and moisture (Kuzuhara et al., 1987; Omenetto and Kaplan, 2010) and is mechanically robust (Altman et al., 2003). Due to its unique block copolymer structure (consisting of large hydrophobic domains and small hydrophilic spacers), silk self-assembles into organized nanoscale crystalline domains (β-sheets) separated by more flexible hydrophilic spacers that produce a stabilizing environment for incorporated proteins and small molecules (Lu et al., 2009). For example, encapsulation of a wide range of water-soluble compounds and proteins (including enzymes and growth factors) in silk biomaterials has been discussed (Numata and Kaplan; Pritchard et al., 2011; Wenk et al., 2011; Pritchard et al., 2012). However, we are not aware that encapsulation of oil, as a dispersion phase or as a solvent for an active agent, in silk biomaterials has been discussed.

### Exemplary Microemulsions of Oil in a Silk Solution (O/W Emulsions)

Manual mixing (gentle shaking for approx. 10 minutes) of an Oil Red O-loaded sunflower oil solution mixed with a silk solution produces stable emulsions of the oil in water (O/W) type (FIG. 2A). Emulsions of sunflower oil in silk were prepared with various silk concentrations (e.g., at ˜2%, ˜4% and ˜6% (w/v)) and volumetric ratios of oil to silk of 1:1, 1:2 and 1:4 and no phase separation was observed for any of the oil in silk emulsions after at least about 48 hours stored at ˜4° C., compared to near total phase separation of 1:1, 1:2 and 1:4 mixtures of sunflower oil and distilled water.

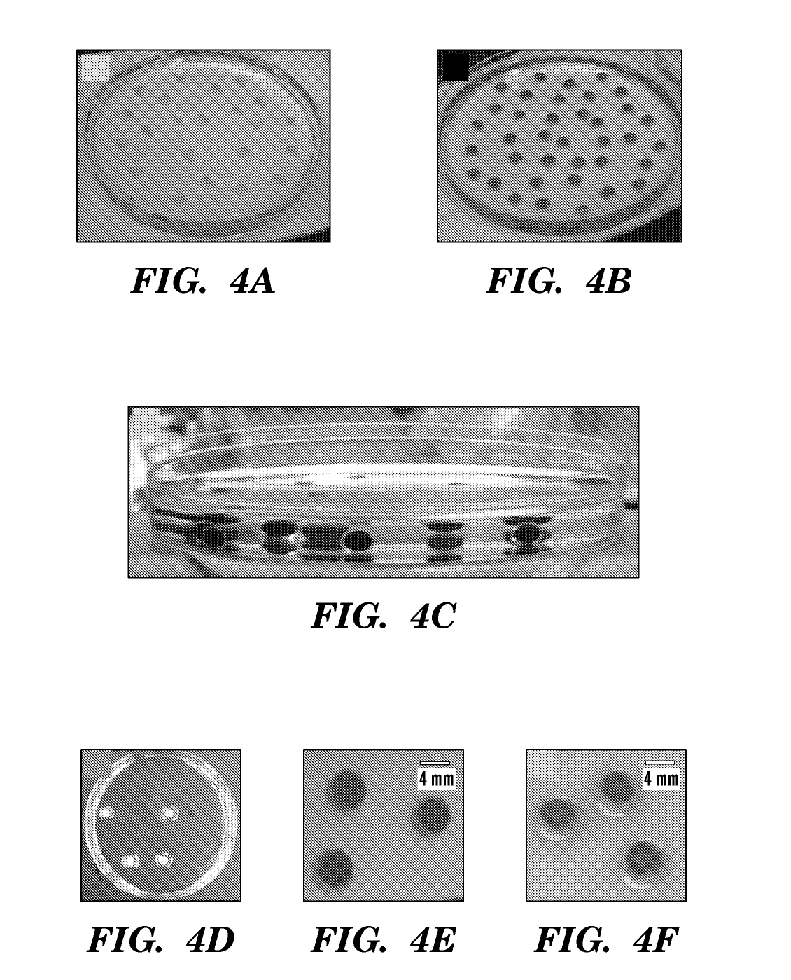

Prior to sonication, an emulsion of sunflower oil containing Oil Red O mixed with ˜7% (w/v) aqueous silk solution in a ˜1:3 (v/v) ratio of oil: silk exhibited an average droplet diameter of 419.5±126.9 μm. Gentle sonication (e.g., 10% amplitude for 5 seconds) of the O/W emulsions reduced the average oil particle diameter to less than 25 μm (a sample of two hundred particles in the image in FIG. 2B measured with ImageJ exhibited an average diameter of 24.6±11.4 μm (but the large number of particles less than 10 μm in diameter were not included in this average as they could not be accurately measured using ImageJ). A microemulsion prepared by sonication of sunflower oil doped with oil red O in silk is shown in FIG. 2B. The microscale oil droplets produced by sonication are stabilized when silk protein is present in the continuous aqueous phase, and can be maintained during self-assembly of silk films during drying (FIG. 3A-3B) or during self-assembly of silk hydrogel networks (FIG. 4B) following sonication.

Following dispersal of oil into the silk solution, e.g., via sonication, the stable emulsion can be treated as a silk solution (without oil) to form different forms of silk articles, for example, as discussed in the art (see, e.g., Omenetto and Kaplan, 2010; Kim et al., 2010; Pritchard et al., 2012; Hofmann et al., 2006; Tsorias et al., 2012). For example, the oil/silk emulsion can be cast into films, rapidly-dissolving films, agent-loaded films for biosensors and diagnostics, and sustained release films for drug-delivery. TGA analysis revealed a slight decrease in thermostability of the silk films loaded with microparticles of oil compared with silk alone (Data not shown). However, self-assembly of the silk into films takes place on both Teflon coated molds and patterned molds, e.g., hologram-patterned molds (FIG. 3A-3B), even when the silk solution contained microparticles of oil. The presence of micron-scale oil droplets in the silk films can render the films opaque rather than transparent, with greater final film opaqueness resulting from higher oil content in the solution (FIG. 3A-3B).

The films were self-assembled by drying overnight (without any further treatment post-drying) at ambient conditions of temperature and pressure, and can be re-dissolved upon exposure to an aqueous medium (e.g., distilled water and phosphate buffered saline), indicating that incorporated oil microparticles can be released upon exposure to an aqueous medium. Alternately, the films can be further treated by a beta-sheeting-inducing process, e.g., water-annealing or water vapor annealing, to increase beta-sheet content in the silk network and thus render the films water insoluble, as have previously been discussed for films cast from silk alone (Jin et al., 2005).

### Silk Particles Produced by Drop-Wise Addition of Sonicated Silk to an Oil Bath

As microemulsions of oil are stable in aqueous silk solutions (O/W emulsion) and do not interfere with silk matrix assembly, it was next sought to evaluate a gentle, aqueous process to produce stable silk particles in oil baths, so that these two components could ultimately be integrated into O/W/O emulsions for microencapsulation. Sonication induces physical crosslinking of silk over tunable timeframes (Wang et al., 2008b; U.S. Pat. No. 8,187,616, the content of which is incorporated herein by reference in its entirety). As a result of this controllable delay between the initiation of the sol-gel transition and the final onset of gelation, sonicated silk still in the solution state aliquoted into oil baths or suspended in self-stabilizing water-in-oil emulsions can complete physical crosslinking without heating or chemical treatment (unlike other emulsion-based processes for preparation of protein microspheres). Stable, physically crosslinked silk spherical particles (e.g., silk macroscale spherical particles) were produced, for example, by sonicating a ˜6-7%, 30 minute degumming time, silk solution for approx. 30-45 seconds at an amplitude of 15%, mixing in solutions of distilled water containing model water-soluble small molecule compounds (e.g., doxorubicin or food coloring) and aliquoting the sonicated silk-drug mixture into a sunflower oil bath. In the oil bath, the aqueous silk droplets are held in a spherical conformation until gelation completes (FIG. 4C). FIG. 4A shows sonicated silk solution in the oil bath prior to the completion of gelation and FIG. 4D shows the same silk droplets after overnight incubation in the oil bath: once crosslinking of the silk network is complete, the silk droplets transition from translucent (FIG. 4A) to opaque and retain their spherical shape when removed from the oil bath (FIG. 4D).

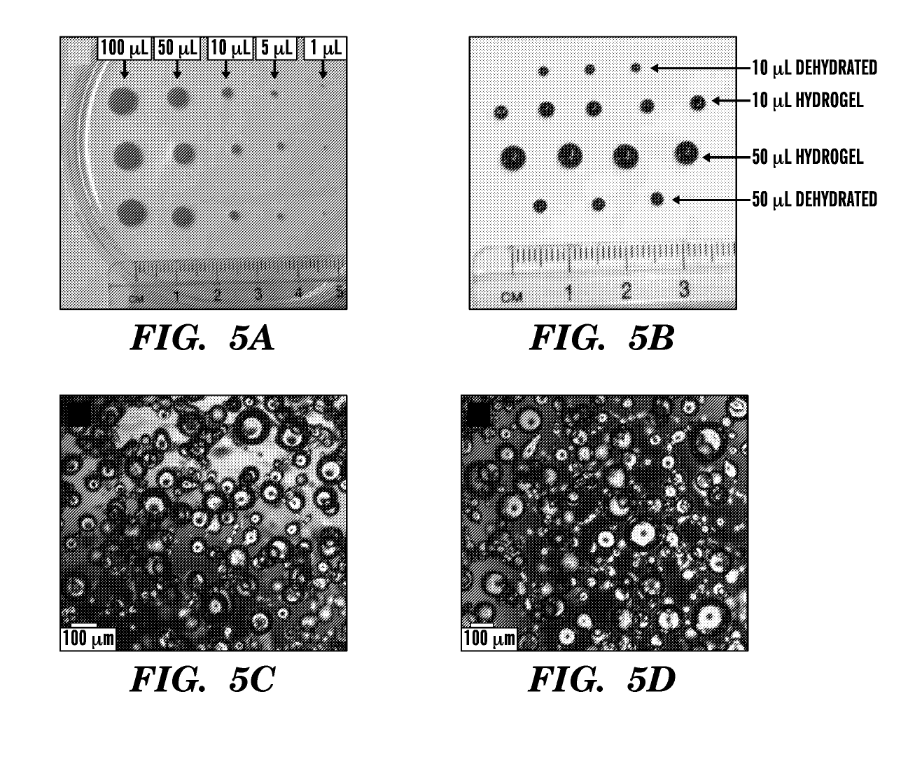

Sonication-induced microemulsion of Oil Red O loaded sunflower oil into silk was then added dropwise into the oil bath (FIG. 4B), which in turn produces crosslinked silk spherical particles with fine, microscale oil particles suspended throughout, resulting in a red coloration of the final silk macroparticle (FIG. 4E). Dehydration of physically crosslinked silk macroparticles by drying overnight at ambient conditions produces smaller, dense, pellet-like particles (oil-loaded in FIG. 4F and water-soluble dye loaded in FIG. 5B).

An extrusion-like process is characterized by precise control of particle size and composition loading due to the pipetting of controlled volumes of a known composition into an oil bath. FIG. 5A shows silk hydrogel macroparticles produced by pipetting sonicated silk solution (loaded with doxorubicin post-sonication) in various volume-size droplets (e.g., from 100 μL down to 1 μL) into the sunflower oil bath. Microparticles produced by pipetting 10 μL or 50 μL of sonicated silk solution (loaded with food coloring post-sonication) and the denser, firmer, smaller particles that result when the hydrogel macroparticles are dehydrated overnight at ambient conditions are shown in FIG. 5B.

The average diameter of silk hydrogel microspheres prepared from 10 μL of sonicated silk solution loaded with dye was about 2.8±0.2 mm prior to drying, and decreased to 1.9±0.3 mm after drying. The average diameter of silk hydrogel microspheres prepared from 50 μL of sonicated silk solution loaded with dye was about 4.6±0.1 mm prior to during, and decreased to 2.3±0.1 mm after drying. Smaller silk microparticles (average volume less than 1 μL) were produced by dispersing silk into oil (W/O emulsion) using sonication (FIGS. 5C-5D). In some embodiments, microfluidics can be used to produce even smaller, more tightly controlled silk particles using the above-described approach (silk sonication followed by dropwise addition to an oil bath), as has been described for other biomaterial microparticles (Chu et al., 2007; Tan and Takeuchi, 2007; Ren et al., 2010).

In addition to varying size and loading, these physically cross-linked silk particles can be further manipulated through post-crosslinking treatments. For example, the crosslinked silk particles can be (1) maintained in a rubbery, hydrated gelled state, (2) dehydrated to produce dense, hardened matrices (FIG. 4F and FIG. 5B) or (3) freeze-dried to produce dry, porous, sponge-like material (Kluge et al., 2010). These different spherical silk particles (all produced using gentle and food-safe processes) span a wide range of material properties and sizes, suitable for a diverse array of potential applications.

Oil-Encapsulated Silk Microparticles Derived from O/W/O Emulsions

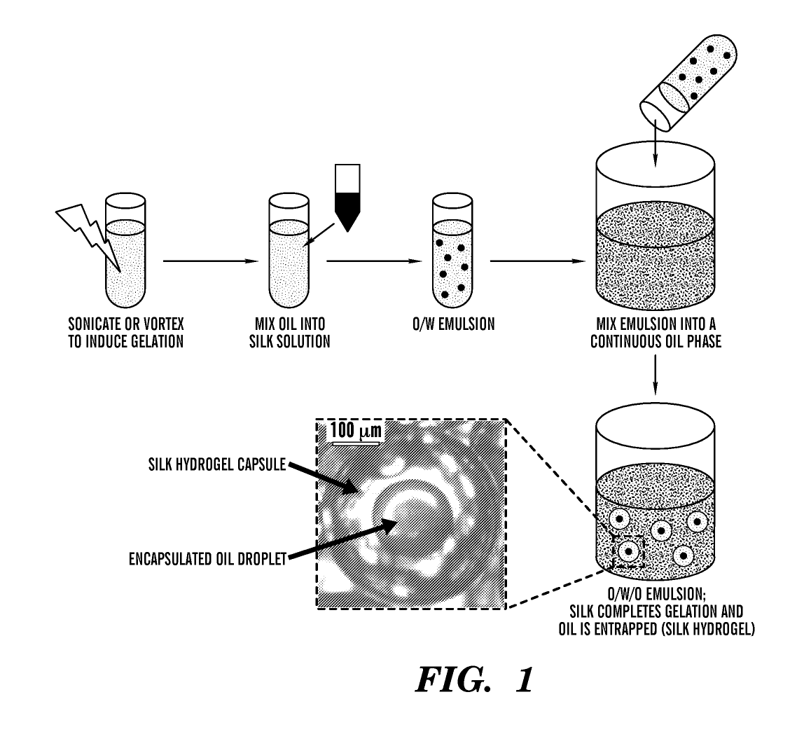

Based on stabilization of emulsified microscale oil droplets in aqueous silk solution and sonicated silk formation of macroscale hydrogel particles in oil baths, microparticles were prepared with a double emulsion of the type O1/W/02 where 01 is the oil of interest to encapsulate (e.g., sunflower oil loaded with Oil Red O presented in this Example), W is an aqueous sol-gel silk solution (e.g., produced by sonicating a silk solution) and 02 is an oil bath (e.g., sunflower oil bath) in which the silk particle are to be dispersed. The silk solution comprising the water phase is sonicated such that it remains in the solution phase long enough to perform the double emulsion, then completes crosslinking, thereby encapsulating the interior oil phase (schematic representation of this process shown in FIG. 1). The silk also acts as a natural emulsion stabilizer, preventing the interior oil phase (loaded with an agent of interest) from separating and leeching the agent into the continuous oil phase. Morphology of O/W/O emulsions prepared from sonicated silk of varied silk composition and sonication treatment was examined with light microscopy, and diffusivity of the silk encapsulating matrices was evaluated by measuring absorbance at 518 nm of the external oil bath (an indicator of Oil Red O diffusing from the internal oil phase of the silk particle into the external continuous oil phase).

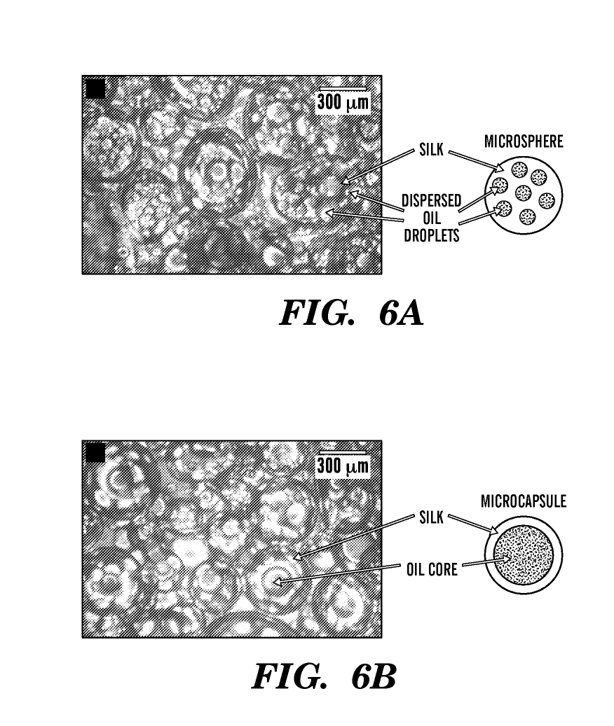

O/W/O emulsions prepared with ˜60 minute degumming time regenerated silk fibroin solution are shown in FIGS. 6A-6B. Using the higher concentration of an aqueous silk solution in the water phase (e.g., ˜6% w/v) can produce a dispersion of oil droplets suspended throughout the silk sphere (this encapsulation configuration is termed a microsphere, also called a matrix system (Kuang et al., 2010)) (FIG. 6A). Use of a lower concentration of an aqueous silk solution (e.g., ˜3% w/v) to prepare the emulsions can result in a microcapsule configuration (also called a reservoir system (Kuang et al., 2010), where one large oil droplet surrounded by a silk capsule is incorporated in each individual particle. This demonstrates that the concentration of the silk can, in part, impact the morphology of the oil-encapsulating microparticle. Without wishing to be bound by theory, the increased viscosity and/or increased protein concentration of silk (e.g., ˜6% (w/v)) may be able to prevent individual droplets from coalescing into a single core droplet as observed with lower concentrations of silk (e.g., ˜3% (w/v)) in O/W/O emulsions.

Increased sonication intensity can accelerate the silk gelation process (Wang et al., 2008). Without wishing to be bound by theory, increased sonication amplitude and/or duration can increase the viscosity of the silk solution. The viscosity of the silk solution can impact particle morphology and/or the permeability of silk as an encapsulant material. Representative images of O/W/O emulsions produced using ˜6% (w/v) silk prepared using a 30 minute degumming time are shown in FIGS. 7A-7D. Compared with the lower viscosity silk emulsions (e.g., using ˜60 min degummed silk solution), the silk particles are less spherical and oil encapsulation appears less regular. When sonication intensity increases (e.g., ˜10% for ˜15 seconds in FIGS. 7A-7B, compared to ˜15% for ˜15 seconds in FIGS. 7C-7D), the resulting silk particles are even more elongated and irregular. Without wishing to be bound by theory, the shorter degumming time combined with the increased sonication intensity may cause premature crosslinking, preventing the silk in the emulsion from incorporating an interior oil droplet and/or adopting a spherical conformation.

During the preparation of microcapsules, material composition and/or diffusivity of the encapsulating matrix material can, in part, determine the retention degree of core agents (Gharsallaoui et al., 2007). At higher solution viscosities, absorbance at 518 nm (an indicator of the Oil Red O content) of the external oil phase (e.g., the sunflower oil bath) decreases, indicating the permeability of the silk capsule to the Oil Red O in the internal oil phase (and consequent “loss” of agent loaded in the internal phase) can decrease as the viscosity of the silk solution in the double emulsion increases. Compared with an aqueous phase of plain distilled water, unsonicated silk can reduce loss of an agent (e.g., Oil Red O) loaded in the internal oil phase to the external oil phase (FIG. 8A). When silk concentration is held constant and sonication treatment is held constant, Oil Red O loss to the external phase decreases with decreasing degumming time (increasing silk solution viscosity) (FIG. 8B). Similarly, when silk solution concentration and degumming time are held constant (˜6% (w/v), ˜30 minute degumming time in FIG. 8C; and ˜6% (w/v), ˜60 minute degumming time in FIG. 8D), but sonication intensity increases (e.g., by amplitude or duration or both), Oil Red O loss generally decreases (with the exception of ˜6% (w/v) ˜30 minute degumming time silk exhibiting no change in Oil Red O loss for unsonicated silk solution compared with silk solution sonicated for ˜15 seconds at an amplitude of ˜15%, possibly because this sonication treatment does not significantly increase viscosity).

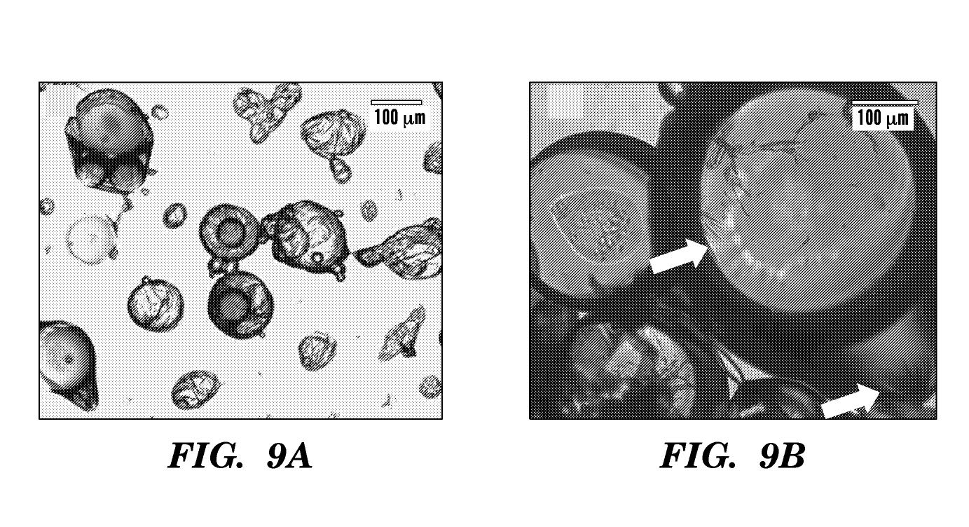

The sunflower oil bath as the continuous, external oil phase in O/W/O emulsions prepared with distilled water containing no silk as the water phase exhibited the highest absorbance at 518 nm (0.442±0.014), indicating the greatest loss of Oil Red O from the internal oil capsule into the continuous oil phase. The continuous oil phases in O/W/O emulsions with unsonicated aqueous silk fibroin solution prepared using a 60 minute and 30 minute degumming time as the water phase had absorbance values at 518 nm of 0.12±0.001 and 0.076±0.001, respectively. The presence of silk in the water phase reduces Oil Red O diffusing into the oil phase (as compared to using water alone as the water phase) (FIG. 8A), indicating that silk encapsulation can provide a barrier to Oil Red O diffusion into the external oil phase. The increase in viscosity of the silk solution (e.g., increasing fragment length of silk in the silk solution by using a shorter degumming time) can further increase retention of an agent in the interior oil core (FIG. 8B). In addition to silk processing parameters, Oil Red O retention in the interior oil core can also be controlled by sonication treatment and concentration (w/v) of the silk solution in the water phase (FIGS. 8C-8D, Table 1). In addition, morphology of the silk O/W/O emulsions indicate that the silk in the aqueous layer assembles into a capsule around the interior oil phase: puckering and wrinkling of the silk “skin” are apparent (FIGS. 9A-9B).

TABLE 1
Absorbance at 518 nm of an external oil phase in an O/W/O
emulsion with a water phase comprising an aqueous silk
solution with varied properties (e.g., degumming duration
and silk concentration) exposed to varied sonication
treatment (treatment duration and amplitude).

| Silk Properties | | Sonication | | |
|---|---|---|---|---|
| Degumming | Silk Con- | Treatment | | Absorbance at 518 nm |
| Duration | centration | Ampli- | Duration | of external oil phase |
| (min) | (w/v) | tude | (sec) | (sunflower oil bath) |
| 60 | 6% | None | None | 0.12 ± 0.001 |
| | 6% | 15% | 30 | 0.098 ± 0.003 |
| | 6% | 15% | 45 | 0.063 ± 0.002 |
| | 3% | 15% | 30 | 0.082 ± 0.002 |
| 30 | 6% | None | None | 0.076 ± 0.001 |
| | 6% | 10% | 15 | 0.076 ± 0.001 |
| | 6% | 15% | 15 | 0.061 ± 0.001 |
| | 3% | 15% | 30 | 0.055 ± 0.001 |
| | 3% | 15% | 15 | 0.072 ± 0.016 |

Gentle, food-safe, aqueous methods for preparing oil-encapsulated silk biomaterials described herein can be used in various applications, e.g., in food or pharmaceutical products where protection, stabilization and/or controlled release are required. Many chemotherapy drugs, steroids, hormones and antibiotics/antifungals are oil soluble but not highly water soluble and thus currently have to be administered with formulation additives like cremaphor or ethanol, which have side-effects in patients.

In one embodiment, the inventors demonstrated encapsulation of sunflower oil, which represents the ability to encapsulate lipids alone (which can benefit from stabilization effects of encapsulation), but also models use of lipids as solvents in which hydrophobic substances such as volatile aromatic compounds (e.g., but not limited to, flavors and fragrances) and lipophilic vitamins and drugs can be solubilized for storage and delivery (Gharsallaoui et al., 2007). The encapsulation system described herein can be used in controlled release/drug delivery applications. Given the gentle, non-toxic, food-safe nature of the encapsulation process (e.g., films and spheres can be prepared at ambient conditions of temperature and pressure, stable emulsions produced without secondary emulsifiers or chemical crosslinking agents), the process described herein can be used for storage and delivery of any agent that can be dissolved in the oil, e.g., but not limited to, flavors, fragrances, food additives, oils and oil-soluble compounds. Silk films prepared with oil in silk microemulsions can also be used for integrating oil-soluble diagnostic agents, e.g., indicator dyes, into diagnostic silk film based platforms.

In some embodiments, the oil-encapsulated silk compositions described herein can be used, for example, in pharmaceutical industry, food and consumer product industry, vendors that sell materials or ingredients (e.g., fragrances, food additives or flavors) to the food and consumer product industry, producers of vitamins, supplements and probiotics; as well as in delivering nutritional supplements, vitamins, etc. to developing world settings where refrigeration is limited to address nutritional deficiencies.

In addition to applications in food, cosmetics, consumer products and medicine, a stable dispersion of oil throughout a protein network can be more physiologically representative than a simple protein hydrogel in modeling tissues with high lipid content, such as the brain.

### Exemplary Materials and Methods

Materials.

Cocoons of Bombyx mori silkworm silk were purchased from Tajima Shoji Co., LTD (Sumiyoshicho, Naka-ku, Yokohama, Japan). Sunflower oil, doxorubicin and Oil Red O were purchased from Sigma Aldrich (St. Louis, Mo.). Limonene was provided by Firmenich (Newark, N.J.).

Silk Solution and Materials Preparation.

Silk fibroin solution was prepared from B. mori cocoons as previously described (Sofia et al., 2001). Briefly, cocoons were boiled for either 30 min or 60 min in a solution of 0.02 M Na₂CO₃ and rinsed, then dried at ambient conditions overnight. The dried fibroin was solubilized in a 9.3 M aqueous LiBr solution at 60° C. for 2-4 h, yielding a 20% (w/v) solution. LiBr was then removed from the silk by dialyzing the solution against distilled water for 2.5 days using Slide-a-Lyzer dialysis cassettes (MWCO 3,500, Pierce Thermo Scientific Inc., Rockford, Ill.). Silk fibroin concentration was determined by evaporating water from a solution sample of known volume and massing using an analytical balance. Silk solutions were stored at 4-7° C. before use.

Silk Film Casting.

Silk films were cast as previously described (Hofmann et al., 2006). Briefly, silk solution was aliquoted into Teflon coated molds or patterned molds, then dried overnight at ambient conditions. Oil-loaded silk films were prepared by sonicating oil into silk solution of the desired concentration at various volumetric ratios of oil: silk using a Branson Digital Sonifier 450 at, e.g., ˜10-15% amplitude for, e.g., ˜5 seconds, then aliquoting and casting as described.

Sonication-Induced Silk Gelation.

Sonication-induced gelation was carried out as previously described in Wang et al., 2008b, and U.S. Pat. No. 8,187,616. For example, a silk solution of the desired concentration and prepared with the degumming duration of interest was sonicated using a Branson Digital Sonifier 450 at ˜10-15% amplitude for varied duration (the various conditions of silk concentration, degumming duration and sonication amplitude and duration are specified throughout the results section). Emulsions were prepared with sonicated or unsonicated silk as described above.

Thermogravimetric Analysis.

Thermogravimetric analysis (TGA) (TA Instruments Q500) was used to measure weight changes of silk films assembled from 1% w/v silk fibroin solutions. TGA curves were obtained under nitrogen atmosphere with a gas flow of 50 mL/min. Analysis was first performed by heating the sample from 25° C. to 600° C. at a rate of 2° C./min. Silk film weight loss was recorded as a function of temperature.

### Example 2

### Films Prepared from Oil-in-Silk Microemulsions—Dissolution and Applications Thereof

Silk films cast and dried overnight at room temperature and ambient conditions that receive no additional beta-sheet-inducing treatment can dissolve rapidly upon exposure to an aqueous environment, such as immersion in buffer (FIG. 10) or when brought into contact with a moist tissue, e.g., a brain tissue, as previously described for ultrathin electronics mounted onto dissolvable silk film substrates (Kim et al., 2010): these patterned films exhibited spontaneous conformal wrapping when applied to the soft, curvilinear surface of the brain tissue. Rapid dissolution of films loaded with a dye and release of the dye from the films occur when the films are immersed in ˜37° C. buffer (FIG. 10). Dissolvable silk films loaded with an active agent (e.g., ˜0.5, 0.25 or 0.125 mg of adenosine per 0.2 mm² film) released the majority of the drug load (approx. 80%) within 15 minutes of exposure to 37° C. phosphate buffered saline (PBS) (Data not shown).

Oil-loaded silk films that were self-assembled by drying overnight at ambient conditions of temperature and pressure re-dissolved upon exposure to distilled water or phosphate buffered saline, thus releasing the incorporated oil and any agent carried in the oil, if any. The capacity of water soluble silk films loaded with oil micro-droplets to re-dissolve upon exposure to aqueous media indicates that not only can the oil-encapsulated silk compositions be used as a storage platform, e.g., for oil-soluble active agents such as therapeutics and nutrients, but can also be used in the cosmetic and food industries, where in some embodiments, the compositions described herein can comprise an optical pattern, e.g., but not limited to, a hologram, iridescence, and reflector pattern. For example, silk films containing microemulsions of flavor-loaded oils can dissolve and release the encapsulated flavor once applied on the tongue or to the inside the cheek. Similarly, fragrance loaded untreated silk films can re-dissolve if applied to slightly dampened skin. Patterning of the silk films can further enhance the consumer's experience. Examples of patterned prototypes were demonstrated in microemulsions of fragrance-loaded oils in silk (FIGS. 3A-3B and FIGS. 11A-11B). For example, the oil-silk microemulsion can be casted on a hologram mold, a plastic sheeting with an iridescent surface, or a reflector-patterned silicone mold, and the resulting silk-based material can retain the optical property (e.g., hologram, iridescence, light reflection).

Because the films can be treated post-drying to cross-link silk fibroin, in some embodiments, oil-soluble compounds (e.g., the ones relevant for use in diagnostic devices) can be integrated into above-described silk platforms for diagnostic applications using similar approaches described herein.

### Example 3

### Hydrogel Silk Spheres (“Silk Pearls”)—Loading and Applications Thereof

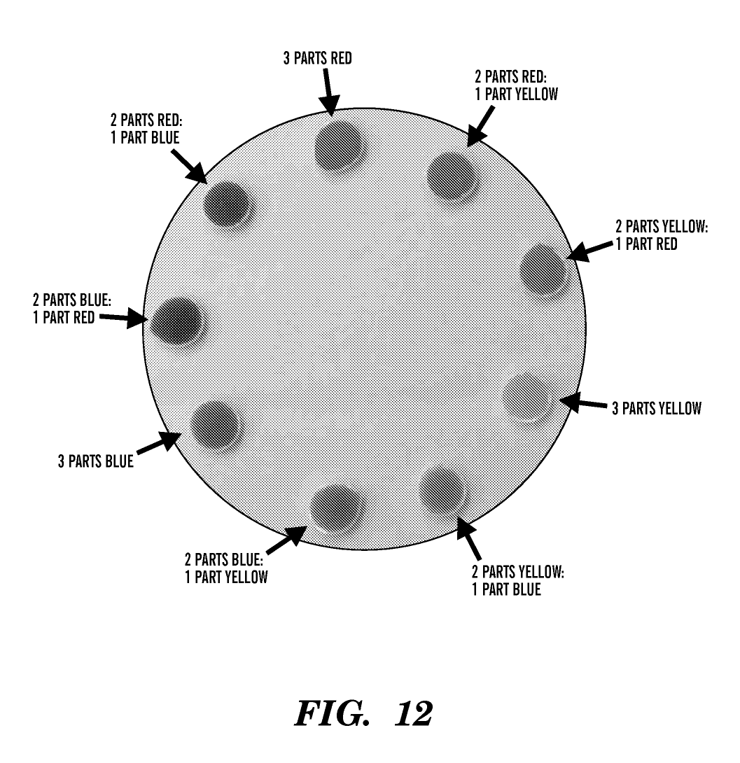

Tunable hydrogel silk spheres with controllable sizes has been described earlier. These cross-linked “silk pearls” can be prepared from microemulsions of oil in silk or loaded with water soluble compounds. Controlling size/diameter of the spheres and/or optional post-crosslinking treatments can be used to extend functionality of the silk compositions described herein. For example, hydrogel silk pearls using varied ratios of food coloring demonstrates controlled loading of the spheres (FIG. 12). Because the preparation involves extrusion of the silk solution into oil baths and the volume and composition of the solution are controlled, encapsulation efficiency of an agent to be loaded in an oil phase and/or silk phase can be up to 100% (unlike other microencapsulation approaches, where compound is frequently lost during processing). The high control and efficiency of loading is demonstrated by the food-coloring loaded silk hydrogel sphere prototypes.

Because these silk hydrogel pearls are stable but soft, they can be used, for example, in food products (e.g., comparable to tapioca pearls), bubble tea and vitamins (e.g., oil-soluble/water-insoluble vitamins and nutritional supplements such as fish oil, beta-carotene and vitamin E). Medication encapsulated in silk hydrogel pearls can represent an alternative administration format for patients who have difficulty swallowing. Using silk instead of gelatin in food products and medication delivery formats can offer the added advantage of alleviating the pathogen transmission concerns associated with use of mammalian sources. Because silk hydrogels are biocompatible and can promote survival of encapsulated cells (Wang et al., 2008), these hydrogel pearls can also be used for products containing probiotic bacteria. In addition, silk compositions can also improve stability during storage (e.g., products with probiotics generally currently require refrigeration) and offer at least some degree of protection during exposure to the harsh environment of the stomach, improving the likelihood of the probiotic bacteria reaching their target site of action further along the gastrointestinal tract.

### REFERENCES

Altman G H, Horan R L, Lu H, Moreau J, Martin I, Richmond J C, Kaplan D L. Silk matrix for tissue engineered anterior cruciate ligaments. Biomaterials 2002; 23:4131-4141.
Arshady R. Albumin microspheres and microcapsules: methodology of manufacturing techniques. J Control Release 1990; 14:111-131.
Baranauskienė R, Venskutonis P R, Dewettinck K, Verhé R. Properties of oregano (Origanum vulgare L.), citronella (Cymbopogon nardus G.) and marjoram (Majorana hortensis L.) flavors encapsulated into milk protein-based matrices. Food Research International. 2006; 39:413-425.
Bayraktar O, Malay O, Özgarip Y, Batigiin A. Silk fibroin as a novel coating material for controlled release of theophylline. Eur J Pharm Biopharm 2005; 60:373-381.
Berthier D L, Schmidt I, Fieber W, Schatz C, Furrer A, Wong K, Lecommandoux S. Controlled release of volatile fragrance molecules from PEO-b-PPO-b-PEO block copolymer micelles in ethanol-water mixtures. Langmuir 2010; 26:7953-7961.
Chourpa I, Ducel V, Richard J, Dubois P, Boury F. Conformational modifications of a gliadin and globulin proteins upon complex coacervates formation with gum Arabic as studied by Raman microspectroscopy. Biomacromolecules 2006; 7:2616-2623.
Chu L-Y, Utada A S, Shah R K, Kim J-W, Weitz D A. Controllable monodisperse multiple emulsions. Angew Chem Int Ed 2007; 46:8970-8974.
Desobry S A, Netto F M, Labuza T P. Comparison of spray-drying, drum-drying and freeze-drying for β-carotene encapsulation and preservation. Journal of Food Science 1997; 62:1158-1162.
Diab T, Pritchard E M, Uhrig B E, Boerckel J D, Kaplan D L, Guldberg R E. A silk hydrogel-based delivery system of bone morphogenetic protein for the treatment of large bone defects. J Mech Behav Biomed Mater. In press
Dickinson E. Double emulsions stabilized by food biopolymers. Food Biophysics 2011; 6:1-11,
Edris A, Bergnståhl B. Encapsulation of orange oil in a spray dried double emulsion. Nahrung 2001; 45:133-137.
Elzoghby A O, Samy W M, Elgindyoghby N A, Samy W M, Elgindy N A. Albumin-based nanoparticles as potential controlled release drug delivery systems. J Control Release 2012; 157:168-182.
Esposito E, Cortesi R, Nastruzzi C. Gelatin microspheres: influence of preparation parameters and thermal treatment on chemico-physical and biopharmaceutical properties. Biomaterials 1996; 17:2009-2020.
Gharsallaoui A, Roudaut G, Chambin O, Voilley A, Saurel R. Applications of spray-drying in microencapsulation of food ingredients: An overview. Food Research International 2007; 40:1107-1121.
Gibbs B F, Kermasha S, Alli I, Mulligan C N. Encapsulation in the food industry: a review. Int J Food Sci Nutr 1999; 50:213-224.
Gouin S. Microencapsulation: industrial appraisal of existing technologies and trends. Trends in Food Sci Technol 2004; 15:330-347.
Hanawa T, Watanabe A, Tsuchiya T, Ikoma R, Hidaka M, Sugihara M. New oral dosage form for elderly patients. II. release behavior of benfotiamine from silk fibroin gel. Chem Pharm Bull 1995; 43:872-876.
Hofmann S, Wong Po Foo C T, Rossetti F, Textor M, Vunjak-Novakovic G, Kaplan D L, Merkle H P, Meinel L. Silk fibroin as an organic polymer for controlled drug delivery. J Control Release 2006; 111: 219-227.
Horan R L, Antle K, Collette A L, Wang Y, Huang J, Moreau J E, Volloch V, Kaplan D L, Altman G H. In vitro degradation of silk fibroin. Biomaterials 2005; 26:3385-3393
Imsombut T, Srisuwan Y, Srihanam P, Baimark Y. Genipin-cross-linked silk fibroin microspheres prepared by the simple water-in-oil emulsion solvent diffusion method. Powder Technol 2010; 203:603-608.
Ishii A, Furukawa M, Matsushima A, Kodera Y, Yamada A, Kanai H, Inada Y. Alteration of properties of natural pigments by conjugation with fibroin or polyethylene glycol. Dyes and Pigments 1995; 27:211-217.
Jayakrishnan A, Knepp W A, Goldberg E P. Casein microspheres: preparation and evaluation as a carrier for controlled drug delivery. International Journal of Pharmaceutics 1994; 106:221-228.
Jin H-J, Park J, Karageorgiou V, Kim U J, Valluzzi R, Cebe P, Kaplan D L. Water-insoluble silk films with reduced β-sheet content. Adv Funct Mater 2005; 15:1241-1247.
Jun-xia X, Hai-yan Y, Jian Y. Microencapsulation of sweet orange oil by complex coacervation with soybean protein isolate/gum Arabic. Food Chemistry 2011; 125:1267-1272.
Kanakdande D, Bhosale R, Singhal R S. Stability of cumin oleoresin microencapsulated in different combination of gum arabic, maltodextrin and modified starch. Carbohydrate Polymers 2007; 67:536-541.
Kim D-H, Viventi J, Amsden J J, Xiao J, Vigeland L, Kim Y-S, Blanco J A, Panilaitis B, Frechette E S, Contreras D, Kaplan D L, Omenetto F G, Huang Y, Hwang K-C, Zakin M R, Litt B, Rogers J A. Dissolvable films of silk fibroin for ultrathin, conformal bio-integrated electronics. Nat Mater 2010; 9:511-517.
Kluge J A, Rosiello N C, Leisk G G, Kaplan D L, Dorfmann A L. The consolidation behavior of silk hydrogels. J Mech Behav Biomed Mater 2010; 3:278-89.
Kuang S S, Oliveira J C, Crean A M. Microencapsulation as a tool for incorporating bioactive ingredients into food. Crit Rev Food Sci Nutr 2010; 50:951-968.
Leal-Egaña A, Scheibel T. Silk-based materials for biomedical applications. Biotechnol Appl Biochem 2010; 55:155-167.
Madene A, Jacquot M, Scher J, Desobry S. Flavour encapsulation and controlled release—a review. Int J Food Sci Technol 2006; 41:1-21.
Matsumoto A, Chen J, Collette A L, Kim U-J, Altman G H, Cebe P, Kaplan D L. Mechanisms of silk fibroin sol-gel transitions. J Phys Chem B 2006; 110:21630-21638.
Meinel L, Hofmann S, Karageorgiou V, Kirker-Head C, McCool J, Gronowicz G, Zichner L, Langer R, Vunjak-Novakovic, Kaplan D L. The inflammatory responses of silk films in vitro and in vivo. Biomaterials 2005; 26:147-155.
Numata K, Kaplan D L. Silk-based delivery systems of bioactive molecules. Adv Drug Deliv Rev. 2010; 62:1497-1508.
Omenetto F G, Kaplan D L. New opportunities for an ancient material. Science 2010; 329:528-531.
Ouali L, Léon G, Normand V, Johnsen H, Dyrli A, Schmid R, Benczedi D. Mechanism of Romascone® release from hydrolyzed vinyl acetate nanoparticles: thermogravimetric method. Polym Adv Technol 2006; 17:45-52.
Panilaitis B, Altman G, Chen J, Jin H J, Karageorgiou V, Kaplan, D L. Macrophage responses to silk. Biomaterials 2003; 24:3079-3085.
Pritchard E M, Dennis P B, Omenetto F, Naik R R, Kaplan D L. Physical and chemical aspects of stabilization of compounds in silk. Biopolymers 2012. 97: 479-498.
Pritchard E M, Kaplan, D L. Silk fibroin biomaterials for controlled release drug delivery. Expert Opin Drug Deliv 2011; 8:1-15.
Qian J, Liu Y, Liu H, Yu T, Deng J. An amperometric new methylene blue N-mediating sensor for hydrogen peroxide based on regenerated silk fibroin as an immobilization matrix for peroxidase. Anal Biochem 1996; 236:208-214.
Ren P-W, Ju X-J, Xie R, Chu L-Y. Monodisperse alginate microcapsules with oil core generated from a microfluidic device. Journal of Colloid and Interface Science 2010; 343:392-395.
Sofia S, McCarthy M B, Gronowicz G, Kaplan D L. Functionalized silk-based biomaterials for bone formation. J Biomed Mater Res 2001; 54:139-148.
Sohail A, Turner M S, Coombes A, Bostrom T, Bhandari B. Survivability of probiotics encapsulated in alginate gel microbeads using a novel impinging aerosols method. Int J Food Microbiol 2011; 145:162-168.
Tan W-H, Takeuchi S. Monodisperse alginate hydrogel microbeads for cell encapsulation. Adv Mater 2007; 19:2696-2701.
Wang X, Kluge J A, Leisk G G, Kaplan D L. Sonication-induced gelation of silk fibroin for cell encapsulation. Biomaterials 2008b; 29:1054-1064.
Wang Y, Kim H-J, Vunjak-Novakovic G, Kaplan D L. Stem cell-based tissue engineering with silk biomaterials. Biomaterials 2006; 27:6064-6082.
Wang Y, Rudym D D, Walsh A, Abrahamsen L, Kim H J, Kim H S. In vivo degradation of three-dimensional silk fibroin scaffolds. Biomaterials 2008a; 29: 3415-3428.
Wenk E, Merkle H P, Meinel L. Silk fibroin as a vehicle for drug delivery applications. J Control Release 2011; 150:128-141.
Wray L S, Hu X, Schmidt D, Kaplan D L. Effect of processing on silk-based biomaterials:
reproducibility and biocompatibility. J Biomed Mater Res B Appl Biomater 2011; 99:89-101.
Zhang X, Reagan M R, Kaplan D L. Electrospun silk biomaterial scaffolds for regenerative medicine. Adv Drug Deliv Rev 2009; 61:988-1006.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

What is claimed is:

1. A silk particle comprising at least two immiscible phases, a first immiscible phase comprising a silk-based material and a second immiscible phase comprising an active agent, wherein the first immiscible phase encapsulates the second immiscible phase and the second immiscible phase excludes a liposome.

2. The silk particle of claim 1, wherein the second immiscible phase comprises a lipid component.

3. The silk particle of claim 2, wherein the lipid component comprises oil.

4. The silk particle of any of claims 1-3, wherein the second immiscible phase forms a single compartment.

5. The silk particle of any of claims 1-3, wherein the second immiscible phase forms a plurality of compartments.

6. The silk particle of claim 4 or 5, wherein the size of the compartment or compartments ranges from about 1 nm to about 1000 μm, or from about 5 nm to about 500 μm.

7. The silk particle of any of claims 1-6, wherein the active agent present in the second immiscible phase comprises a hydrophobic or lipophilic molecule.

8. The silk particle of claim 7, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof.

9. The silk particle of any of claims 1-8, wherein the silk-based material comprises an additive.

10. The silk particle of claim 9, wherein the additive is selected from the group consisting of biocompatible polymers; plasticizers (e.g., glycerol); stimulus-responsive agents; active agents, small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

11. The silk particle of claim 9 or 10, wherein the additive is in a form of a particle (e.g., a nanoparticle or microparticle, including a plasmonic particle), a fiber, a tube, powder or any combinations thereof.

12. The silk particle of any of claims 9-11, wherein the additive comprises a silk material, e.g., silk particles, silk fibers, micro-sized silk fibers, unprocessed silk fibers, and any combinations thereof.

13. The silk particle of any of claims 1-12, wherein the second immiscible phase encapsulates a third immiscible phase.

14. The silk particle of any of claims 1-13, wherein the silk-based material is present in a form of a hydrogel.

15. The silk particle of any of claims 1-14, wherein the silk-based material is present in a dried state or lyophilized.

16. The silk particle of claim 15, wherein the lyophilized silk matrix is porous.

17. The silk particle of any of claims 1-16, wherein the silk-based material in the first immiscible phase is soluble in an aqueous solution.

18. The silk particle of any of claims 1-17, wherein beta-sheet content in the silk-based material is adjusted to an amount sufficient to enable the silk-based material to resist dissolution in an aqueous solution.

19. The silk particle of any of claims 1-18, wherein the size of the silk particle ranges from about 10 nm to about 10 mm, or from about 50 nm to about 5 mm.

20. A composition comprising a plurality of lipid compartments encapsulated in a silk-based material.

21. The composition of claim 20, wherein the size of the lipid compartments ranges from about 1 nm to about 1000 μm, or from about 5 nm to about 500 μm.

22. The composition of claim 20 or 21, wherein the volumetric ratio of the lipid compartments to the silk-based material ranges from about 1000:1 to about 1:1000, from about 500:1 to about 1:500, or from about 100:1 to about 1:100.

23. The composition of any of claims 20-22, wherein the silk-based material is in a form selected from the group consisting of a film, a sheet, a gel or hydrogel, a mesh, a mat, a non-woven mat, a fabric, a scaffold, a tube, a slab or block, a fiber, a particle, powder, a 3-dimensional construct, an implant, a foam or a sponge, a needle, a lyophilized material, a porous material, a non-porous material, and any combinations thereof.

24. The composition of any of claims 20-23, wherein the silk-based material comprises a film.

25. The composition of any of claims 20-24, wherein the silk-based material comprises a scaffold.

26. The composition of any of claims 20-25, wherein the silk-based material comprises an optical pattern.

27. The composition of claim 26, wherein the optical pattern comprises a hologram or an array of patterns that provides an optical functionality.

28. The composition of any of claims 20-27, wherein the lipid compartments further comprise an active agent.

29. The composition of claim 20-28, wherein the active agent comprises a hydrophobic or lipophilic molecule.

30. The composition of claim 29, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof.

31. The composition of any of claims 20-30, wherein the silk-based material comprises an additive.

32. The composition of claim 31, wherein the additive is selected from the group consisting of biocompatible polymers; plasticizers (e.g., glycerol); stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

33. The composition of claim 31 or 32, wherein the additive is in a form selected from the group consisting of a particle, a fiber, a tube, a film, a gel, a mesh, a mat, a non-woven mat, a powder, and any combinations thereof.

34. The composition of any of claims 31-33, wherein the additive comprises a silk material, e.g., silk particles, silk fibers, micro-sized silk fibers, unprocessed silk fibers, and any combinations thereof.

35. A composition comprising a collection of silk particles of any of claims 1-19.

36. The composition of claim 35, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, a scaffold, or any combinations thereof.

37. The composition of claim 35 or 36, wherein the composition is formulated for use in a pharmaceutical product.

38. The composition of claim 35 or 36, wherein the composition is formulated for use in a cosmetic product.

39. The composition of claim 35 or 36, wherein the composition is formulated for use in a personal care product.

40. The composition of claim 35 or 36, wherein the composition is formulated for use in a food product.

41. A storage-stable composition comprising a silk particle of any of claims 1-19 or a composition of any of claims 20-40, wherein the active agent present in the second immiscible phase of the silk particle, or a hydrophobic or lipophilic molecule present in the lipid components retains at least about 30% of its original bioactivity after the composition is (a) subjected to at least one freeze-thaw cycle, or (b) maintained for at least about 24 hours at a temperature of about room temperature or above, or (c) both (a) and (b).

42. The composition of claim 41, wherein the composition is maintained under exposure to light.

43. The composition of claim 41 or 42, wherein the composition is maintained at a relative humidity of at least about 10%.

44. The composition of any of claims 41-43, wherein the silk-based material of the silk particle or the composition is in a dried-state.

45. A method of producing a silk particle comprising:
a. providing an emulsion of non-aqueous droplets dispersed in a silk solution undergoing a sol-gel transition (where the silk solution remains in a mixable state); and
b. contacting a pre-determined volume of the emulsion with a non-aqueous phase, whereby the silk solution forms in the non-aqueous phase a silk particle entrapping at least one of the non-aqueous droplets therein.

46. The method of claim 45, wherein the sol-gel transition last for about at least 1 hour, or at least about 2 hours.

47. The method of claim 45 or 46, wherein the sol-gel transition of the silk solution is induced by sonication.

48. The method of claim 47, where the sonication is performed at an amplitude of about 5% to about 20%, or about 10% to about 15%.

49. The method of claim 47 or 48, wherein the sonication duration lasts for about 15 sec to about 60 sec, or from about 30 sec to about 45 sec.

50. The method of any of claims 45-49, wherein the silk solution has a concentration of about 1% (w/v) to about 15% (w/v), or about 2% (w/v) to about 7% (w/v).

51. The method of any of claims 45-50, further comprising adding an active agent into the silk fibroin solution undergoing a sol-gel transition.

52. The method of any of claims 45-51, wherein the non-aqueous droplets further comprise a hydrophobic or lipophilic molecule.

53. The method of claim 52, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), a small molecule, or any combinations thereof.

54. The method of any of claims 45-53, wherein the emulsion is produced by adding a non-aqueous, immiscible phase into the silk solution, thereby forming the non-aqueous droplets dispersed in the silk solution.

55. The method of any of claims 45-54, wherein the pre-determined volume of the emulsion substantially corresponds to a desirable size of the silk particle.

56. The method of any of claims 45-55, further comprising isolating the silk particle from the non-aqueous phase.

57. The method of any of claims 45-56, further comprising subjecting the silk particle to a post-treatment.

58. The method of claim 57, wherein the post-treatment further induces a conformational change in silk fibroin in the particle.

59. The method of claim 58, wherein said inducing conformational change comprises one or more of lyophilization or freeze-drying, water annealing, water vapor annealing, alcohol immersion, sonication, shear stress, electrogelation, pH reduction, salt addition, air-drying, electrospinning, stretching, or any combination thereof.

60. The method of any of claims 57-59, wherein the post-treatment comprises freeze-drying the silk particle.

61. A method comprising a step of: maintaining a composition, wherein the composition comprises at least one lipid compartment encapsulated in a silk-based material and at least one active agent distributed in said at least one lipid compartment, and wherein the active agent retains at least about 30% of its original bioactivity after the composition is (a) subjected to at least one freeze-thaw cycle, or (b) maintained for at least about 24 hours at a temperature of about room temperature or above, or (c) both (a) and (b).

62. The method of claim 61, wherein the composition is maintained for at least about 1 month.

63. A method comprising a step of: maintaining a composition, wherein the composition comprises at least one lipid compartment encapsulated in a silk-based material and at least one active agent distributed in said at least one lipid compartment, and wherein the silk-based material is permeable to said at least one active agent such that the active agent is released through the silk-based material into an ambient surrounding at a pre-determined rate.

64. The method of claim 63, wherein the pre-determined rate is controlled by adjusting an amount of beta-sheet conformation of silk fibroin present in the silk-based material, porosity of the silk-based material, or a combination thereof.

65. The method of claim 63 or 64, wherein the composition is maintained at about room temperature.

66. The method of any of claims 61-65, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.

67. The method of any of claims 61-66, wherein the composition is lyophilized.

68. The method of any of claims 61-67, wherein the composition is maintained at a temperature of about 37° C. or greater.

69. The method of any of claims 61-68, wherein the composition is maintained under exposure to light.

70. The method of any of claims 61-69, wherein the composition is maintained at a relative humidity of at least about 10%.

71. The method of any of claims 61-70, wherein the active agent comprises a hydrophobic or lipophilic active agent.

72. The method of claim 71, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

73. The method of any of claims 61-72, wherein the silk-based material comprises an additive.

74. The method of claim 73, wherein the additive is selected from the group consisting of biocompatible polymers; plasticizers (e.g., glycerol); stimulus-responsive agents; small organic or inorganic molecules; saccharides; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; glycogens or other sugars; immunogens; antigens; an extract made from biological materials such as bacteria, plants, fungi, or animal cells; animal tissues; naturally occurring or synthetic compositions; and any combinations thereof.

75. The method of claim 73 or 74, wherein the additive is in a form selected from the group consisting of a particle, a fiber, a tube, a film, a gel, a mesh, a mat, a non-woven mat, a powder, and any combinations thereof.

76. The method of any of claims 73-75, wherein the additive comprises a silk material, e.g., silk particles, silk fibers, micro-sized silk fibers, unprocessed silk fibers, or any combinations thereof.

77. A method of delivering an active agent comprising applying or administering to a subject a composition comprising a silk-based material, the silk-based material encapsulating at least one lipid compartment with an active agent disposed therein, said silk-based material being permeable to the active agent such that the active agent is released through the silk-based material, at a pre-determined rate, upon application or administration of the composition to the subject.

78. The method of claim 77, wherein the active agent is released to an ambient surrounding.

79. The method of claim 77 or 78, wherein the active agent is released to at least one target cell of the subject.

80. The method of any of claims 77-79, wherein the active agent comprises a hydrophobic or lipophilic active agent.

81. The method of claim 80, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

82. The method of any of claims 77-81, wherein the silk-based material comprises an additive.

83. The method of any of claims 77-82, wherein the composition is applied or administered to the subject topically.

84. The method of claim 83, wherein the composition is applied on a skin of the subject.

85. The method of any of claims 77-82, wherein the composition is applied or administered to the subject orally.

86. A silk particle comprising at least two immiscible phases, a first immiscible phase comprising a silk-based material and a second immiscible phase comprising an active agent, wherein the first immiscible phase encapsulates the second immiscible phase and the second immiscible phase excludes a liposome.

87. The silk particle of claim 86, wherein the second immiscible phase comprises a lipid component.

88. The silk particle of claim 87, wherein the lipid component comprises oil.

89. The silk particle of any of claims 86-88, wherein the second immiscible phase forms a single compartment.

90. The silk particle of any of claims 86-89, wherein the second immiscible phase forms a plurality of compartments.

91. The silk particle of claim 89 or 90, wherein the size of the compartment or compartments ranges from about 1 μm to about 1000 μm, or from about 10 μm to about 500 μm.

92. The silk particle of any of claims 86-91, wherein the active agent present in the second immiscible phase comprises a hydrophobic or lipophilic molecule.

93. The silk particle of claim 92, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

94. The silk particle of any of claims 86-93, wherein the silk-based material comprises an additive.

95. The silk particle of claim 94, wherein the additive comprises a biopolymer, an active agent, a plasmonic particle, glycerol, and any combinations thereof.

96. The silk particle of any of claims 86-95, wherein the second immiscible phase encapsulates a third immiscible phase.

97. The silk particle of any of claims 86-96, wherein the silk-based material is present in a form of a hydrogel.

98. The silk particle of any of claims 86-96, wherein the silk-based material is present in a dried state or lyophilized.

99. The silk particle of claim 98, wherein the lyophilized silk matrix is porous.

100. The silk particle of any of claims 86-99, wherein at least the silk-based material in the first immiscible phase is soluble in an aqueous solution.

101. The silk particle of any of claims 86-99, wherein beta-sheet content in the silk-based material is adjusted to an amount sufficient to enable the silk-based material to resist dissolution in an aqueous solution.

102. The silk particle of any of claims 86-101, wherein the size of the silk particle ranges from about 0.1 mm to about 10 mm, or from about 0.5 mm to about 5 mm.

103. A composition comprising a plurality of lipid compartments encapsulated in a silk-based material.

104. The composition of claim 103, wherein the size of the lipid compartments ranges from about 1 μm to about 1000 μm, or from about 10 μm to about 500 μm.

105. The composition of claim 103 or 104, wherein the volumetric ratio of the lipid compartments to the silk-based material ranges from about 1:1 to about 1:1000, from about 1:5 to about 1:500, or from about 1:10 to about 1:100.

106. The composition of any of claims 103-105, wherein the silk-based material comprises a film.

107. The composition of claim 106, wherein the silk-based material comprises an optical pattern.

108. The composition of claim 107, wherein the optical pattern comprises a hologram or an array of patterns that provides an optical functionality.

109. The composition of any of claims 103-108, wherein the silk-based material comprises a scaffold.

110. The composition of any of claims 103-109, wherein the lipid compartments further comprise an active agent.

111. The composition of claim 110, wherein the active agent comprises a hydrophobic or lipophilic molecule.

112. The composition of claim 111, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

113. The composition of any of claims 103-112, wherein the silk-based material comprises an additive.

114. The composition of claim 113, wherein the additive comprises a biopolymer, an active agent, a plasmonic particle, glycerol, and any combinations thereof.

115. A composition comprising a collection of silk particles of any of claims 86-102.

116. The composition of claim 115, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.

117. The composition of claim 115 or 116, wherein the composition is formulated for use in a pharmaceutical product.

118. The composition of claim 115 or 116, wherein the composition is formulated for use in a cosmetic product.

119. The composition of claim 115 or 116, wherein the composition is formulated for use in a food product.

120. A storage-stable composition comprising a silk particle of any of claims 86-102 or a composition of any of claims 103-119, where the active agent present in the second immiscible phase of the silk particle, or a hydrophobic or lipophilic molecule present in the lipid components retains at least about 30% of its original bioactivity when the composition is (a) subjected to at least one freeze-thaw cycle, or (b) maintained for at least about 24 hours at a temperature of about room temperature or above, or (c) both (a) and (b).

121. The composition of claim 120, wherein the composition is maintained under exposure to light.

122. The composition of claim 120 or 121, wherein the composition is maintained at a relative humidity of at least about 10%.

123. The composition of any of claims 120-122, wherein the cross-linked silk matrix is in a dried-state.

124. A method of producing a silk particle comprising:
a. providing or obtaining an emulsion of non-aqueous droplets dispersed in a silk solution undergoing a sol-gel transition (where the silk solution remains in a mixable state); and
b. contacting a pre-determined volume of the emulsion with a non-aqueous phase, whereby the silk solution entraps at least one of the non-aqueous droplets and gels to form a silk particle dispersed in the non-aqueous phase.

125. The method of claim 124, wherein the sol-gel transition last for about at least 1 hour, or at least about 2 hours.

126. The method of claim 124 or 125, wherein the sol-gel transition of the silk solution is induced by sonication.

127. The method of claim 126, where the sonication is performed at an amplitude of about 5% to about 20%, or about 10% to about 15%.

128. The method of claim 126 or 127, wherein the sonication duration lasts for about 15 sec to about 60 sec, or from about 30 sec to about 45 sec.

129. The method of any of claims 124-128, wherein the silk solution has a concentration of about 1% (w/v) to about 15% (w/v), or about 2% (w/v) to about 7% (w/v).

130. The method of any of claims 124-129, further comprising adding an active agent into the silk fibroin solution undergoing a sol-gel transition.

131. The method of any of claims 124-130, wherein the non-aqueous droplets further comprise a hydrophobic or lipophilic molecule.

132. The method of claim 131, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

133. The method of any of claims 124-132, wherein the emulsion is produced by adding a non-aqueous, immiscible phase into the silk solution, thereby forming the non-aqueous droplets dispersed in the silk solution.

134. The method of any of claims 124-133, wherein the pre-determined volume of the emulsion is a volume corresponding to a desirable size of the silk particle.

135. The method of any of claims 124-134, further comprising isolating the silk particle from the non-aqueous phase.

136. The method of any of claims 124-135, further comprising freeze-drying the silk particle.

137. A method comprising a step of: maintaining a composition, wherein the composition comprises at least one lipid compartment encapsulated a silk-based material and at least one active agent distributed in said at least one lipid compartment, and wherein the active agent retains at least about 30% of its original bioactivity when the composition is (a) subjected to at least one freeze-thaw cycle, or (b) maintained for at least about 24 hours at a temperature of about room temperature or above, or (c) both (a) and (b).

138. The method of claim 137, wherein the composition is maintained for at least about 1 month.

139. A method comprising a step of: maintaining a composition, wherein the composition comprises at least one lipid compartment encapsulated a silk-based material and at least one active agent distributed in said at least one lipid compartment, and wherein the silk-based material is permeable to said at least one active agent such that the active agent is released through the silk-based material into an ambient surrounding at a pre-determined rate.

140. The method of claim 139, wherein the pre-determined rate is controlled by adjusting an amount of beta-sheet conformation of silk fibroin present in the silk-based material, porosity of the silk-based material, or a combination thereof.

141. The method of claim 139 or 140, wherein the composition is maintained at about room temperature.

142. The method of any of claims 137-141, wherein the composition is an emulsion, a colloid, a cream, a gel, a lotion, a paste, an ointment, a liniment, a balm, a liquid, a solid, a film, a sheet, a fabric, a mesh, a sponge, an aerosol, powder, or any combinations thereof.

143. The method of any of claims 137-142, wherein the composition is lyophilized.

144. The method of any of claims 137-143, wherein the composition is maintained at a temperature of about 37° C. or greater.

145. The method of any of claims 137-144, wherein the composition is maintained under exposure to light.

146. The method of any of claims 137-145, wherein the composition is maintained at a relative humidity of at least about 10%.

147. The method of any of claims 137-146, wherein the active agent comprises a hydrophobic or lipophilic active agent.

148. The method of claim 147, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

149. The method of any of claims 137-148, wherein the silk-based material comprises an additive.

150. The method of claim 149, wherein the additive comprises a biopolymer, an active agent, a plasmonic particle, glycerol, and any combinations thereof.

151. A method of delivering an active agent comprising applying or administering to a subject a composition comprising a silk-based material, the silk-based material encapsulating a lipid compartment with an active agent disposed therein, said silk-based material being permeable to the active agent such that the active agent is released through the silk-based material, at a pre-determined rate, upon application or administration of the composition to the subject.

152. The method of claim 151, wherein the active agent is released to an ambient surrounding.

153. The method of claim 151 or 152, wherein the active agent is released to at least one target cell of the subject.

154. The method of any of claims 151-153, wherein the active agent comprises a hydrophobic or lipophilic active agent.

155. The method of claim 154, wherein the hydrophobic or lipophilic molecule comprises a therapeutic agent, a nutraceutical agent, a cosmetic agent, a coloring agent, a probiotic agent, a dye, an aromatic compound, an aliphatic compound (e.g., alkane, alkene, alkyne, cyclo-alkane, cyclo-alkene, and cyclo-alkyne), or any combinations thereof.

156. The method of any of claims 151-155, wherein the silk-based material comprises an additive.

157. The method of claim 156, wherein the additive comprises a biopolymer, an active agent, a plasmonic particle, glycerol, and any combinations thereof.

158. The method of any of claims 151-157, wherein the composition is applied or administered to the subject topically or orally.

159. The method of any of claims 151-158, wherein the composition is applied on skin of the subject.
